SEQUENCE LISTING (11 sequences)

SEQ ID NO: 1 - protein, 5 aa, Artificial Sequence
  Synthetic Construct
DYWMQ

SEQ ID NO: 2 - protein, 17 aa, Artificial Sequence
  Synthetic Construct
TIYPGDGDTGYAQKFQG

SEQ ID NO: 3 - protein, 11 aa, Artificial Sequence
  Synthetic Construct
GDYYGSNSLDY

SEQ ID NO: 4 - protein, 11 aa, Artificial Sequence
  Synthetic Construct
KASQDVSTVVA

SEQ ID NO: 5 - protein, 7 aa, Artificial Sequence
  Synthetic Construct
SASYRYI

SEQ ID NO: 6 - protein, 9 aa, Artificial Sequence
  Synthetic Construct
QQHYSPPYT

SEQ ID NO: 7 - protein, 120 aa, Artificial Sequence [sequence omitted]

SEQ ID NO: 8 - protein, 108 aa, Artificial Sequence
  Synthetic Construct
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKR

SEQ ID NO: 9 - protein, 108 aa, Artificial Sequence
  Synthetic Construct
DIVMAQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKR

SEQ ID NO: 10 - protein, 450 aa, Artificial Sequence
  Synthetic Construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 214 aa, Artificial Sequence
  Synthetic Construct
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2021/0171650 A1
(43) Pub. Date: Jun. 10, 2021

(54) METHODS OF ADMINISTERING ANTI-CD38 ANTIBODY

(71) Applicant: Sanofi, Paris (FR)

(72) Inventors: Heloise AUDAT, Paris (FR); Sylvia MARION, Ormond Beach, FL (US); Frank CAMPANA ZAMBRANO, Sudbury, MA (US)

(21) Appl. No.: 15/931,466

(22) Filed: May 13, 2020

Related U.S. Application Data
(60) Provisional application No. 62/899,088, filed on Sep. 11, 2019.
(60) Provisional application No. 62/860,739, filed on Jun. 12, 2019.
(60) Provisional application No. 62/847,825, filed on May 14, 2019.

(30) Foreign Application Priority Data
Mar. 3, 2020 (EP) EP20305223.8

(51) Int. Cl. C07K 16/28; A61P 35/00; A61K 9/00

(52) U.S. Cl. CPC C07K 16/2896; A61K 2039/505; A61K 9/0019; A61P 35/00

(57) ABSTRACT

Provided are methods of treating a human individual having multiple myeloma that comprise administering to the individual 10 mg/kg isatuximab via intravenous infusion, wherein the volume of each infusion of 10 mg/kg isatuximab is 250 ml. Also provided are methods of treating a human individual having multiple myeloma that comprise administering an anti-CD38 antibody in 28-day cycles, wherein the anti-CD38 antibody is administered on Days 1, 8, 15, and 22 of a first 28-day cycle, wherein the CD38-antibody is administered on Days 1 and 15 of every 28-day cycle following the first 28-day cycle; and wherein the anti-CD38 antibody is administered at a dose of 10 mg/kg or 20 mg/kg.

### CROSS REFERENCE TO RELATED APPLICATIONS

This application claims the priority benefit of European Patent Application No. EP20305223.8, filed Mar. 3, 2020; U.S. Provisional Application No. 62/899,088, filed Sep. 11, 2019; U.S. Provisional Application No. 62/860,739 filed Jun. 12, 2019; and U.S. Provisional Application No. 62/847,825, filed May 14, 2019, the contents of each of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 183952031700SEQLIST.TXT, date recorded: May 12, 2020, size: 11 KB).

### FIELD OF THE INVENTION

The present disclosure relates to methods of treating multiple myeloma by administering an anti-CD38 antibody.

### BACKGROUND

Therapeutic antibodies have improved the options for treating patients with relapsed and/or refractory multiple myeloma (RRMM). However, therapeutic antibodies are typically administered via intravenous infusion, and infusion reactions (IRs) are a commonly reported side effect. Symptoms of IR (e.g., rash, urticarial, flushing, changes in heart rate and/or blood pressure, fever, dyspnea, and/or nausea) require prompt management to avoid severe adverse events, including fatality. Strategies for mitigating the risk of IRs (or resolving mild IRs) include slowing the infusion rate, temporarily interrupting the infusion, and/or splitting the infusion dose over two or more consecutive days. However, lengthy and/or frequent intravenous infusions can be costly, burdensome, and inconvenient for patients, leading to reduced compliance with the treatment regimen. Moreover, lengthy and/or frequent IV infusions require extended hospital stays and longer observation times, thus increasing the workloads of hospital employees. What is needed in the art are safe and effective methods of administering therapeutic antibody for the treatment of RRMM that are also more convenient for patients, physicians, and other medical staff.

### BRIEF SUMMARY

In some embodiments, provided is an anti-CD38 antibody for use in a method of treating a an individual in need thereof, the method comprising administering to the individual at least a first intravenous infusion of the anti-CD38 antibody, wherein the anti-CD38 antibody is administered at a dose of at least 10 mg/kg, wherein the dose of the anti-CD38 antibody is in a volume of 250 ml, and wherein the anti-CD38 antibody comprises (a) a heavy chain variable domain (VH) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (VL) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6). In some embodiments the anti-CD38 antibody (e.g., the administration of the anti-CD38 antibody) is for the treatment of a disease or disorder, optionally wherein the disease or disorder is multiple myeloma. In some embodiments, provided is a method of administering an anti-CD38 antibody to a human individual in need thereof, comprising administering to the individual at least a first intravenous infusion of the anti-CD38 antibody, wherein the anti-CD38 antibody is administered at a dose of at least 10 mg/kg, wherein the dose of the anti-CD38 antibody is in a volume of 250 ml, and wherein the anti-CD38 antibody comprises (a) a heavy chain variable domain (VH) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (VL) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6). In some embodiments, the administration of the anti-CD38 antibody is for the treatment of multiple myeloma. In some embodiments, the anti-CD38 antibody is isatuximab.

In some embodiments, the first intravenous infusion of the anti-CD38 antibody is administered to the individual at an infusion rate of 25 mL/hour for a first hour, and wherein the infusion rate is increased by 25 mL/hour every 30 minutes after the first hour to a maximum infusion rate of 150 mL/hour until the 250 ml volume is infused. In some embodiments, the first infusion of the anti-CD38 antibody is administered to the individual at an infusion rate of 12.5 mL/hour for a first 30 minutes, and wherein the infusion rate is increased by 25 mL/hour every 30 minutes after the first 30 minutes until the 250 ml volume is infused. In some embodiments, the method comprises administering to the individual at least a second intravenous infusion of the anti-CD38 antibody, wherein the anti-CD38 antibody is administered at a dose of at least 10 mg/kg in a volume of 250 ml. In some embodiments, the second intravenous infusion of the anti-CD38 is administered to the individual at an infusion rate of 50 mL/hour for a first 30 minutes, wherein the infusion rate is increased by 50 ml/hr for a second 30 minutes, and wherein the infusion rate is increased by 100 mL/hour every 30 minutes after the second 30 minutes to a maximum infusion rate of 200 mL/hour until the 250 ml volume is infused. In some embodiments, the second intravenous infusion of the anti-CD38 is administered to the individual at an infusion rate of 50 mL/hour for a first 30 minutes, 100 mL/hour for a second 30 minutes, 200 mL for the third 30 minutes, and 300 mL/hour after the third 30 minutes until the 250 ml volume is infused. In some embodiments, the second intravenous infusion of the anti-CD38 is administered to the individual at an infusion rate of 25 mL/hour for a first 30 minutes, and wherein the infusion rate is increased by 50 mL/hour every 30 minutes after the first 30 minutes until the 250 ml volume is infused. In some embodiments, the method comprises administering to the individual at least a third intravenous infusion of the anti-CD38 antibody, wherein the anti-CD38 antibody is administered at a dose of at least 10 mg/kg in a volume of 250 ml. In some embodiments, the third intravenous infusion of the anti-CD38 is administered to the individual at an infusion rate of 200 ml/hour until the 250 ml volume is infused. In some embodiments, the third intravenous infusion of the anti-CD38 is administered to the individual at an infusion rate of 100 ml/hour for a first 30 minutes, and wherein the infusion rate is increased by 50 mL/hour every 30 minutes after the first 30 minutes until the 250 ml volume is infused. In some embodiments, the method comprises administering to the individual a fourth intravenous infusion of the anti-CD38 antibody, wherein the anti-CD38 antibody is administered at a dose of at least 10 mg/kg in a volume of 250 ml. In some embodiments, the fourth intravenous infusion of the anti-CD38 antibody is administered to the individual at an infusion rate of 200 ml/hour until the 250 ml volume infused. In some embodiments, the fourth intravenous infusion of the anti-CD38 antibody is administered to the individual at an infusion rate of 100 ml/hour for a first 30 minutes, and wherein the infusion rate is increased by 50 mL/hour every 30 minutes after the first 30 minutes until the 250 ml volume is infused. In some embodiments, the anti-CD38 antibody is administered in a first 28-day cycle, wherein the first intravenous infusion of the anti-CD38 antibody is administered on Day 1, the second intravenous infusion of the anti-CD38 antibody is administered on Day 8, the third intravenous infusion of the anti-CD38 antibody is administered on Day 15, and the fourth intravenous infusion of the anti-CD38 antibody is administered on Day 22 the first 28-day cycle.

In some embodiments, the method comprises administering to the individual one or more subsequent intravenous infusions of the anti-CD38 antibody following the fourth intravenous infusion, wherein the anti-CD38 antibody is administered at a dose of at least 10 mg/kg in a volume of 250 ml for each of the one or more subsequent intravenous infusions. In some embodiments, each of the one or more subsequent intravenous infusions of the anti-CD38 antibody following the fourth intravenous infusion is administered to the individual at an infusion rate of 200 ml/hour until the 250 ml volume infused. In some embodiments, each of the one or more subsequent intravenous infusions of the anti-CD38 antibody following the fourth intravenous infusion is administered to the individual at an infusion rate of 100 ml/hour for a first 30 minutes, and wherein the infusion rate is increased by 50 mL/hour every 30 minutes after the first 30 minutes until the 250 ml volume is infused. In some embodiments, the anti-CD38 antibody is administered in one or more subsequent 28-day cycles following the first 28-day cycle, wherein each of the one or more subsequent intravenous infusions of the anti-CD38 antibody following the fourth intravenous infusion is administered on Days 1 and 15 of each of the one or more subsequent 28-day cycles following the first 28-day cycle.

In some embodiments, provided is an anti-CD38 antibody for use in a method of treating a an individual in need thereof, the method comprising administering to the individual at least three intravenous infusions of the anti-CD38 antibody, wherein the anti-CD38 antibody is administered at a dose of at least 10 mg/kg, wherein the dose of the anti-CD38 antibody is in a volume of 250 ml, and wherein the anti-CD38 antibody comprises (a) a heavy chain variable domain (VH) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (VL) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6); wherein the first intravenous infusion of the anti-CD38 antibody is administered to the individual at an infusion rate of 25 mL/hour for a first hour, and wherein the infusion rate is increased by 25 mL/hour every 30 minutes after the first hour to a maximum infusion rate of 150 mL/hour until the 250 ml volume is infused; wherein the second intravenous infusion of the anti-CD38 is administered to the individual at an infusion rate of 50 mL/hour for a first 30 minutes, wherein the infusion rate is increased by 50 ml/hr a second 30 minutes, and wherein the infusion rate is increased by 100 mL/hour every 30 minutes after the second 30 minutes to a maximum infusion rate of 200 mL/hour until the 250 ml volume is infused; and wherein the third intravenous infusion of the anti-CD38 is administered to the individual at an infusion rate of 100 ml/hour for a first 30 minutes, and wherein the infusion rate is increased by 50 mL/hour every 30 minutes after the first 30 minutes until the 250 ml volume is infused.

In some embodiments, provided is a method of administering an anti-CD38 antibody to a human individual in need thereof, comprising administering to the individual at least three intravenous infusion of the anti-CD38 antibody, wherein the anti-CD38 antibody is administered at a dose of at least 10 mg/kg, wherein the dose of the anti-CD38 antibody is in a volume of 250 ml, and wherein the anti-CD38 antibody comprises (a) a heavy chain variable domain (VH) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (VL) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6); wherein the first intravenous infusion of the anti-CD38 antibody is administered to the individual at an infusion rate of 25 mL/hour for a first hour, and wherein the infusion rate is increased by 25 mL/hour every 30 minutes after the first hour to a maximum infusion rate of 150 mL/hour until the 250 ml volume is infused; wherein the second intravenous infusion of the anti-CD38 is administered to the individual at an infusion rate of 50 mL/hour for a first 30 minutes, wherein the infusion rate is increased by 50 ml/hr a second 30 minutes, and wherein the infusion rate is increased by 100 mL/hour every 30 minutes after the second 30 minutes to a maximum infusion rate of 200 mL/hour until the 250 ml volume is infused; and wherein the third intravenous infusion of the anti-CD38 is administered to the individual at an infusion rate of 100 ml/hour for a first 30 minutes, and wherein the infusion rate is increased by 50 mL/hour every 30 minutes after the first 30 minutes until the 250 ml volume is infused.

In some embodiments, the anti-CD38 antibody is isatuximab. In some embodiments, the anti-CD38 antibody is for the treatment of a disease or disorder, optionally wherein the disease or disorder is multiple myeloma. In some embodiments, the administration of the anti-CD38 antibody is for the treatment of multiple myeloma. In some embodiments, the method further comprises administering to the individual one or more subsequent intravenous infusions of the anti-CD38 antibody following the third intravenous infusion, wherein the anti-CD38 antibody is administered at a dose of at least 10 mg/kg in a volume of 250 ml for each of the one or more subsequent intravenous infusions. In some embodiments, each of the one or more subsequent intravenous infusions of the anti-CD38 antibody following the third intravenous infusion is administered to the individual at an infusion rate of 200 ml/hour until the 250 ml volume infused.

In some embodiments, provided is an anti-CD38 antibody for use in a method of treating an individual in need thereof, the method comprising administering the anti-CD38 antibody to the individual via intravenous infusion, wherein the anti-CD38 antibody is administered at a dose of at least 10 mg/kg, wherein the volume of each dose of the anti-CD38 antibody is 250 ml, and wherein the anti-CD38 antibody comprises (a) a heavy chain variable domain (VH) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (VL) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6). In some embodiments the anti-CD38 antibody (e.g., the administration of the anti-CD38 antibody) is for the treatment of a disease or disorder, optionally wherein the disease or disorder is multiple myeloma. In some embodiments, provided is method of administering an anti-CD38 antibody to an individual in need thereof, comprising administering the anti-CD38 antibody to the individual via intravenous infusion, wherein the anti-CD38 antibody is administered at a dose of at least 10 mg/kg, wherein the volume of each dose of the anti-CD38 antibody is 250 ml, and wherein the anti-CD38 antibody comprises (a) a heavy chain variable domain (VH) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (VL) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6). In some embodiments, the administration of the anti-CD38 antibody is for the treatment of multiple myeloma. In some embodiments, the anti-CD38 antibody is isatuximab.

In some embodiments, the anti-CD38 antibody is administered in a first 28-day cycle, and the anti-CD38 antibody is administered on Days 1, 8, 15, and 22 of the first 28-day cycle. In some embodiments, the anti-CD38 antibody is administered to the individual via intravenous infusion on Day 1 of the first 28 day cycle) at an infusion rate of 25 mL/hour for a first hour, and the infusion rate is increased by 25 mL/hour every 30 minutes after the first hour to a maximum infusion rate of 150 mL/hour until the 250 ml dose of the anti-CD38 antibody is infused. In some embodiments, the anti-CD38 antibody is administered to the individual via intravenous infusion on Day 1 of the first 28-day cycle) at an infusion rate of 12.5 mL/hour for a first 30 minutes, and the infusion rate is increased by 25 mL/hour every 30 minutes after the first 30 minutes until the 250 ml dose of the anti-CD38 antibody is infused. In some embodiments, the anti-CD38 antibody is administered to the individual via intravenous infusion on Day 8 of the first 28 day cycle at an infusion rate of 50 mL/hour for a first 30 minutes, wherein the infusion rate is increased by 50 ml/hr for the next 30 minutes, and wherein the infusion rate is increased by 100 mL/hour every 30 minutes after the first 60 minutes to a maximum infusion rate of 200 mL/hour until the 250 ml volume is infused. In some embodiments, the anti-CD38 antibody is administered to the individual via intravenous infusion on Day 8 of the first 28 day cycle at an infusion rate of 50 mL/hour for a first 30 minutes, 100 mL/hour for a second 30 minutes, 200 mL for the third 30 minutes, and 300 mL/hour after the third 30 minutes until the 250 ml dose of the anti-CD38 antibody is infused. In some embodiments, the anti-CD38 antibody is administered to the individual via intravenous infusion on Day 8 of the first 28 day cycle at an infusion rate of 25 mL/hour for a first 30 minutes, and the infusion rate is increased by 50 mL/hour every 30 minutes after the first 30 minutes until the 250 ml dose of the anti-CD38 antibody is infused. In some embodiments, the anti-CD38 antibody is administered to the individual via intravenous infusion on Day 15 of the first 28 -day cycle at an infusion rate of 200 ml/hour until the 250 ml dose of the anti-CD38 antibody is infused. In some embodiments, the anti-CD38 antibody is administered to the individual via intravenous infusion on Day 15 of the first 28 day cycle at an infusion rate of 100 ml/hour for a first 30 minutes, and the infusion rate is increased by 50 mL/hour every 30 minutes after the first 30 minutes until the 250 ml dose of the anti-CD38 antibody is infused. In some embodiments, the anti-CD38 antibody is administered to the individual via intravenous infusion on Day 22 of the first 28-day cycle at an infusion rate of 200 ml/hour until the 250 ml dose of the anti-CD38 antibody is infused. In some embodiments, the anti-CD38 antibody is administered to the individual via intravenous infusion on Day 22 of the first 28 day cycle at an infusion rate of 100 ml/hour for a first 30 minutes, and the infusion rate is increased by 50 mL/hour every 30 minutes after the first 30 minutes until the 250 ml dose of the anti-CD38 antibody is infused.

In some embodiments, the anti-CD38 antibody is further administered in one or more subsequent 28-day cycles, and the anti-CD38 antibody is administered at a dose of at least 10 mg/kg on Days 1 and 15 of each subsequent 28-day cycle, the volume of each dose of the anti-CD38 antibody administered in the one or more subsequent cycles is 250 ml. In some embodiments, the anti-CD38 antibody is administered to the individual via intravenous infusion on Day 1 of each subsequent 28-day cycle at an infusion rate of 200 ml/hour until the 250 ml dose of the anti-CD38 antibody is infused. In some embodiments, the anti-CD38 antibody is administered to the individual via intravenous infusion on Day 1 of each subsequent 28-day cycle at an infusion rate of 100 ml/hour for a first 30 minutes, and the infusion rate is increased by 50 mL/hour every 30 minutes after the first 30 minutes until the 250 ml dose of the anti-CD38 antibody is infused. In some embodiments, the anti-CD38 antibody is administered to the individual via intravenous infusion on Day 15 of each subsequent 28-day cycle at an infusion rate of 200 ml/hour until the 250 ml dose of the anti-CD38 antibody is infused. In some embodiments, the anti-CD38 antibody is administered to the individual via intravenous infusion on Day 15 of each subsequent 28-day cycle at an infusion rate of 100 ml/hour for a first 30 minutes, and the infusion rate is increased by 50 mL/hour every 30 minutes after the first 30 minutes until the 250 ml dose of the anti-CD38 antibody is infused.

In some embodiments, provided is an anti-CD38 antibody for use in a method of treating an individual in need thereof, the method comprising safely administering to the individual at least a first dose of the anti-CD38 antibody via intravenous infusion, wherein the anti-CD38 antibody is administered at a dose of at least 10 mg/kg in a volume of 250 ml, wherein the first dose is infused over a duration of about 1.5 and about 6.5 hours, and wherein the anti-CD38 antibody comprises (a) a heavy chain variable domain (VH) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (VL) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6). In some embodiments the anti-CD38 antibody (e.g., the administration of the anti-CD38 antibody) is for the treatment of a disease or disorder, optionally wherein the disease or disorder is multiple myeloma. In some embodiments, provided is a method of safely administering an anti-CD38 antibody to a human individual in need thereof, comprising administering to the individual at least a first dose of the anti-CD38 antibody via intravenous infusion, wherein the anti-CD38 antibody is administered at a dose of at least 10 mg/kg in a volume of 250 ml, wherein the first dose is infused over a duration of about 1.5 and about 6.5 hours, and wherein the anti-CD38 antibody comprises (a) a heavy chain variable domain (VH) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (VL) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6). In some embodiments, the administration of the anti-CD38 antibody is for the treatment of multiple myeloma. In some embodiments, the anti-CD38 antibody is isatuximab.

In some embodiments, at least a second dose of the anti-CD38 antibody is administered to the individual via intravenous infusion, wherein the anti-CD38 antibody is administered at a dose of at least 10 mg/kg in a volume of 250 ml, wherein the second dose is infused over a duration of about 0.5 and about 3.5 hours. In some embodiments, at least a third dose of the anti-CD38 antibody is administered to the individual via intravenous infusion, wherein the anti-CD38 antibody is administered at a dose of at least 10 mg/kg in a volume of 250 ml, wherein the third dose is infused over a duration of about 0.5 and about 1.5 hours. In some embodiments each dose of at least 10 mg/kg anti-CD38 antibody in a volume of 250 ml following the third dose is infused over a duration between about 0.5 hours and about 1.5 hours. In some embodiments of any of the methods herein, the dose of anti-CD38 antibody (e.g., isatuximab) is 10 mg/kg or 20 mg/kg

In some embodiments, the administration of the anti-CD38 antibody does not result in the individual experiencing an infusion reaction (IR). In some embodiments, the administration of the anti-CD38 antibody does not result in the individual experiencing an IR greater than Grade 1 in severity. In some embodiments, the administration of the anti-CD38 antibody does not result in the individual experiencing an IR of Grade 2 or greater in severity. In some embodiments, the individual does not receive premedication with one or more of an analgesic, an antacid, an anti-inflammatory agent, an antihistamine prior for the purpose of preventing or minimizing an infusion reaction prior to the administration of the anti-CD38 antibody via intravenous infusion.

In some embodiments, provided is an intravenous (IV) bag containing 250 mls of a 10 mg/kg dose of an anti-CD38 antibody (e.g., isatuximab). In some embodiments, the 10 mg/kg dose of the anti-CD38 antibody (e.g., isatuximab) is calculated based on the weight of the patient to whom the anti-CD38 antibody is to be administered. In some embodiments, the anti-CD38 antibody (e.g., isatuximab) is diluted from a concentrated formulation (e.g., a formulation described herein) into 0.9% sodium chloride, 5% glucose, or 5% dextrose. In some embodiments, the bag contains between about 360 mg and about 1600 mg, between about 450 mg and about 16000 mg, between about 450 mg and 1140 mg, or between about 450 mg and about 910 mg, including any range in between these values. In some embodiments, the intravenous bag containing the 10 mg/kg dose of the anti-CD38 antibody in the volume of 250 ml further comprises 0.9% sodium chloride or 5% dextrose.

In some embodiments, provided is an anti-CD38 antibody for use in a method of treating multiple myeloma an individual, comprising administering to the individual an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6), pomalidomide, and dexamethasone, wherein the anti-CD38 antibody is administered in 28-day cycles; wherein the anti-CD38 antibody is administered on Days 1, 8, 15, and 22 of a first 28-day cycle; wherein the anti-CD38 antibody is administered on Days 1 and 15 of every 28-day cycle following the first 28-day cycle; and wherein the anti-CD38 antibody is administered at a dose of 10 mg/kg or 20 mg/kg.

In some embodiments, provided is a method of treating a human individual having multiple myeloma, comprising administering to the individual an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6), pomalidomide, and dexamethasone, wherein the anti-CD38 antibody is administered in 28-day cycles; wherein the anti-CD38 antibody is administered on Days 1, 8, 15, and 22 of a first 28-day cycle; wherein the anti-CD38 antibody is administered on Days 1 and 15 of every 28-day cycle following the first 28-day cycle; and wherein the anti-CD38 antibody is administered at a dose of 10 mg/kg or 20 mg/kg.

In some embodiments, the individual has at least one high-risk cytogenetic abnormality selected from: 17p deletion, 4(4; 14) translocation, and t(14;16) translocation. In some embodiments, the individual has at least two high-risk cytogenetic abnormalities.

In some embodiments, the multiple myeloma is relapsed/refractory multiple myeloma. In some embodiments, the individual was refractory to the most recent prior therapy for multiple myeloma. In some embodiments, the individual is refractory to lenalidomide. In some embodiments, the individual's most recent prior therapy for multiple myeloma was lenalidomide. In some embodiments, the individual is refractory to a proteasome inhibitor. In some embodiments, the individual's most recent prior therapy was a proteasome inhibitor. In some embodiments, the proteasome inhibitor is selected from the group consisting of: bortezomib, carfilzomib, marizomib, oprozomib, and ixazomib. In some embodiments, the individual received prior therapy with lenalidomide and a proteasome inhibitor, and the lenalidomide were administered to the individual in combination. In some embodiments, the individual received prior therapy with lenalidomide and a proteasome inhibitor, and the lenalidomide were administered to the individual separately (e.g., each during a different prior line of therapy). In some embodiments, the individual has received at least two prior therapies for multiple myeloma. In some embodiments, the individual has received at least three prior therapies for multiple myeloma.

In some embodiments, the individual has a respiratory, thoracic, and/or mediastinal disorder. In some embodiments, the respiratory disorder is chronic obstructive pulmonary disorder (COPD). In some embodiments, the respiratory disorder is asthma. In some embodiments, the respiratory disorder is bronchospam.

In some embodiments, the anti-CD38 antibody is administered to the individual in conjunction with at least one additional agent. In some embodiments, the at least one additional agent comprises an immunomodulatory drug. In some embodiments, the immunomodulatory drug is lenalidomide or pomalidomide. In some embodiments, the at least one additional agent comprises a proteasome inhibitor. In some embodiments, the proteasome inhibitor is bortezomib, carfilzomib, marizomib, oprozomib, and ixazomib. In some embodiments, the at least one additional agent comprises a corticosteroid. In some embodiments, the corticosteroid is dexamethasone.

In some embodiments, provided is a kit comprising isatuximab for treating an individual having multiple myeloma according to a method of an embodiment provided herein.

### DESCRIPTION OF THE FIGURES

The patent or application file contains at least one drawing executed in color. Copies of this patent or patent application publication with color drawing(s) will be provided by the Office upon request and payment of the necessary fee.

FIG. 1 provides a schematic of the study design of the clinical trial described in Example 1A.

FIG. 2 shows the percentage of patients who experienced an infusion reaction (IR) of Grade 2 or Grade 3 by infusion number in the clinical trial described in Example 1B (isatuximab infusion rate measured as ml/h) as compared to the percentage of patients who experienced an infusion reaction (IR) of Grade 2 or Grade 3 by infusion number in a parallel trial in which isatuximab was administered according to a standard infusion protocol (infusion rate measured as mg/h).

FIG. 3 provides the median duration (hours) of isatuximab infusions in the clinical trial described in Example 1B as compared to the median duration (hours) of isatuximab infusion in a parallel clinical trial in which isatuximab was administered according to a standard infusion protocol.

FIG. 4 provides the Logit Emax model that best described the relationship between isatuximab exposure and ORR in the modeling studies described in Example 2. CT4W=Ctrough at 4 weeks.

FIG. 5 provides the distribution of responders and non-responders by CT4W quartiles in the modeling studies described in Example 2. BOR=Best overall response.

FIG. 6 provides the predicted relationship between the probability of response and CT4W in the modeling studies described in Example 2. BMPC=bone marrow plasma cell percent.

FIG. 7 provides a disease model, including an exposure-driven tumor growth inhibiting (TGI) and a pharmacokinetics model from the modeling studies described in Example 2. Serum-M protein kinetics were adequately described by the exposure-driven TGI model. Dropouts were accounted for using a joint model.

FIG. 8 provides a comparison of model-predicted and observed longitudinal serum M-protein kinetics for the indicated dosing regimens.

FIGS. 9A-9B provide clinical trial simulations of isatuximab monotherapy with the indicated dosing regimens. 5000 clinical trials of 100 patients each were simulated. FIG. 9A shows simulated overall response rates (RR) using the E-R model from the modeling studies described in Example 2. The 100 patients in the clinical trial simulations were resampled from 168 actual patients, assuming they received the same dose level for each simulated trial. FIG. 9B shows simulated percent changes of M-protein from at eight weeks after baseline using the Disease M-protein model from the modeling studies described in Example 2. Each clinical trial simulation was based on 122 actual patients, assuming they received the same dose level.

FIG. 10 provides the patient dispositions for Phase 1 and Phase 2 of the study described in Example 3. SD=standard deviation.

FIG. 11 provides the pharmacokinetic profile of isatuximab (mean isatuximab concentration) in Phase 1, cycle 1 of the study described in Example 3.

FIG. 12 provides a Swimmer plot for best response and time on treatment in Phase 2 of the study described in Example 2. Patients were treated with an isatuximab dose of 20 mg/kg QW/Q2W. AE, adverse event; CR, complete response; MR, minimal response; NE, not evaluable; ORR, overall response rate; PD, progressive disease; PR, partial response; SD, stable disease; UNCPD, unconfirmed PD; VGPR, very good partial response.

FIGS. 13A-13B provide Kaplan-Meier plots of progression-free survival (FIG. 13A) and overall survival (FIG. 13B) of patients treated with isatuximab at 20 mg/kg QW/Q2W in the study described in Example 3.

FIG. 14 shows the relationship between CD38 receptor density and clinical response in patients receiving isatuximab at a dose of 10 mg/kg QWx4/Q2W or 20 mg/kg QWx4/Q2W.

FIG. 15 shows a Kaplan-Meier plot of progression-free survival (PFS) of patients administered with isatuximab, pomalidomide, and dexamethasone, where the isatuximab was administered to the patients from a fixed infusion volume of 250 ml.

FIG. 16 shows a Kaplan-Meier plot of overall survival (OS) of patients administered with isatuximab, pomalidomide, and dexamethasone, where the isatuximab was administered to the patients from a fixed infusion volume of 250 ml.

### DETAILED DESCRIPTION

Therapeutic antibodies for the treatment of multiple myeloma (including relapsed and/or refractory multiple myeloma “RRMM”) have the potential to cause infusion reactions (IRs) when administered intravenously. IRs present with a variety of symptoms, including, e.g., rash, urticarial, flushing, changes in heart rate and/or blood pressure, fever, dyspnea, and/or nausea, etc.) during or within 24 hours of intravenous infusion. IRs can range in severity from mild to life-threatening, but in all cases, prompt attention and an immediate response to the patient's initial symptoms are essential. Numerous strategies, e.g., including slowing infusion rate, interrupting infusion, and splitting the infusion over two or more consecutive days, have been adopted to mitigate and/or prevent IRs. However, such strategies can inconvenience patients and increase medical costs. Further, extended hospital stays and frequent hospital visits increase the workloads of hospital staff.

Provided herein are methods of treating multiple myeloma (e.g., RRMM) that comprise administering an effective amount of an anti-CD38 antibody (e.g., 10 mg/kg isatuximab) to an individual via intravenous infusion, wherein the volume of each anti-CD38 antibody infusion is 250 ml. Applicant found that such fixed volume of anti-CD38 antibody (e.g., isatuximab) can be infused rapidly, thus significantly decreasing the duration without detriment to patient safety.

Also provided herein are methods of treating multiple myeloma (e.g., RRMM) that comprise administering 20 mg/kg of an anti-CD38 antibody (e.g., isatuximab) to an individual on each of Days 1, 8, 15, and 22 of a first 28-day cycle, and further administering 20 mg/kg of an anti-CD38 antibody (e.g., isatuximab) to the individual on each of Days 1 and 15 of every subsequent 28-day cycle following the first 28-day cycle.

### Definitions

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se. “Sustained response” refers to the sustained effect on preventing or delaying progression of a disease (e.g., multiple myeloma) and/or improving one or more response criteria after cessation of a treatment. For example, response to treatment for multiple myeloma may be measured according to the criteria in Kumar et al. (2016) “International Myeloma Working Group consensus criteria for response and minimal residual disease assessment in multiple myeloma.” Lancet Oncol. 17(8): e328-e346) and Durie et al. (2006) “International uniform response criteria for multiple myeloma. Leukemia. 20: 1467-1473. (See also Table 14 herein). In some embodiments, the sustained response has a duration at least the same as the treatment duration, at least 1.5×, 2.0×, 2.5×, or 3.0× length of the treatment duration.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

As used herein, the term “treatment” refers to clinical intervention designed to alter the typical course of the disease or cell (e.g., cancer cell) being treated during the course of clinical pathology. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals.

As used herein, “delaying progression of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

An “effective amount” is at least the minimum amount required to effect a measurable improvement or prevention of a particular disorder. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “in conjunction with” refers to administration of one treatment modality in addition to another treatment modality. As such, “in conjunction with” refers to administration of one treatment modality before, during, or after administration of the other treatment modality to the individual.

A “subject” or an “individual” for purposes of treatment refers to any animal classified as a mammal, including humans, domestic and farm animals, and zoo, sports, or pet animals, such as dogs, horses, cats, cows, etc. Preferably, the mammal is human.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. As used herein, the term “overall response rate” or “ORR” refers to the proportion of patients with stringent complete response (sCR), complete response (CR), very good partial response (VGPR), and partial response (PR), as assessed by the IRC using the IMWG response criteria described in Kumar et al. (2016) “International Myeloma Working Group consensus criteria for response and minimal residual disease assessment in multiple myeloma.” Lancet Oncol. 17(8): e328-e346 and Durie et al. (2006) “International uniform response criteria for multiple myeloma. Leukemia. 20: 1467-1473. See also Table 14.

The following description sets forth exemplary methods, parameters and the like. It should be recognized, however, that such description is not intended as a limitation on the scope of the present disclosure but is instead provided as a description of exemplary embodiments.

### Overview

Provided herein are methods for treating or delaying the progression of multiple myeloma in an individual who has received at least two prior therapies for multiple myeloma (e.g., such as lenalidomide and a proteasome inhibitor). In some embodiments, the methods comprise administering to the individual 10 mg/kg of an anti-CD38 antibody (e.g., isatuximab) via intravenous infusion, wherein each infusion of 10 mg/kg of the anti-CD38 antibody (e.g., isatuximab) is in a 250 ml volume. In some embodiments, the individual does not experience an IR (or experiences only a mild IR) during or following the infusion. In some embodiments, the method comprises administering 20 mg/kg of an anti-CD38 antibody (e.g., isatuximab) to the individual on each of Days 1, 8, 15, and 22 of a first 28-day cycle. In some embodiments, the method comprises further administering an anti-CD38 antibody (e.g., isatuximab) to the individual in one or more subsequent 28-day cycles following the first 28 day-cycle. In some embodiments, the method comprises further administering 20 mg/kg of an anti-CD38 antibody (e.g., isatuximab) to the individual on each of Days 1 and 15 of every subsequent 28-day cycle following the first 28-day cycle.

### Anti-CD38 Antibodies

In some embodiments, the anti-CD38 antibody binds to human CD38. In some embodiments, the anti-CD38 antibody is a human antibody, a humanized antibody, or a chimeric antibody. In some embodiments, the anti-CD38 antibody comprises (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6). In some embodiments, the anti-CD38 antibody comprises a heavy chain variable domain (V_{H}) that comprises an amino acid sequence that is at least 90% identical (e.g., at least any one of 91%, 92%, 94%, 95%, 96%, 97%, 98%, or 99%, including any range between these values) to SEQ ID NO: 7. Additionally or alternatively, in some embodiments, the anti-CD38 antibody comprises a light chain variable domain (V_{L}) that comprises an amino acid sequence that is at least 90% identical (e.g., at least any one of 91%, 92%, 94%, 95%, 96%, 97%, 98%, or 99%, including any range between these values) to SEQ ID NO: 8 or SEQ ID NO: 9. In some embodiments, the anti-CD38 antibody comprises a V_{H} that comprises SEQ ID NO: 7 and a V_{L} that comprises SEQ ID NO: 8 or SEQ ID NO: 9.

(SEQ ID NO: 7)
QVQLVQSGAE VAKPGTSVKL SCKASGYTFT DYWMQWVKQR
PGQGLEWIGT IYPGDGDTGY AQKFQGKATL TADKSSKTVY
MHLSSLASED SAVYYCARGD YYGSNSLDYW GQGTSVTVSS
(SEQ ID NO: 8)
DIVMTQSHLS MSTSLGDPVS ITCKASQDVS TVVAWYQQKP
GQSPRRLIYS ASYRYIGVPD RFTGSGAGTD FTFTISSVQA
EDLAVYYCQQ HYSPPYTFGG GTKLEIKR
(SEQ ID NO: 9)
DIVMAQSHLS MSTSLGDPVS ITCKASQDVS TVVAWYQQKP
GQSPRRLIYS ASYRYIGVPD RFTGSGAGTD FTFTISSVQA
EDLAVYYCQQ HYSPPYTFGG GTKLEIKR

In some embodiments, the anti-CD38 antibody is isatuximab (CAS Registry Number: 1461640-62-9). Isatuximab, also known as hu38SB19 and SAR650984, is an anti-CD38 antibody described in WO 2008/047242 and U.S. Pat. No. 8,153,765, the contents of both of which are incorporated by reference herein in their entirety.

The heavy chain of isatuximab comprises the amino acid sequence:

(SEQ ID NO: 10)
QVQLVQSGAE VAKPGTSVKL SCKASGYTFT DYWMQWVKQR
PGQGLEWIGT IYPGDGDTGY AQKFQGKATL TADKSSKTVY
MHLSSLASED SAVYYCARGD YYGSNSLDYW GQGTSVTVSS
ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKVEP KSCDKTHTCP PCPAPELLGG
PSVFLFPPKP KDTLMISRTP EVTCVVVDVS HEDPEVKFNW
YVDGVEVHNA KTKPREEQYN STYRVVSVLT VLHQDWLNGK
EYKCKVSNKA LPAPIEKTIS KAKGQPREPQ VYTLPPSRDE
LTKNQVSLTC LVKGFYPSDI AVEWESNGQP ENNYKTTPPV
LDSDGSFFLY SKLTVDKSRW QQGNVFSCSV MHEALHNHYT
QKSLSLSPG

and the light chain of isatuximab comprises the amino acid sequence:

(SEQ ID NO: 11)
DIVMTQSHLS MSTSLGDPVS ITCKASQDVS TVVAWYQQKP
GQSPRRLIYS ASYRYIGVPD RFTGSGAGTD FTFTISSVQA
EDLAVYYCQQ HYSPPYTFGG GTKLEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

The anti-CD38 antibodies may be produced using recombinant methods. For recombinant production of an anti-antigen antibody, nucleic acid encoding the antibody is isolated and inserted into a replicable vector for further cloning (amplification of the DNA) or for expression. DNA encoding the antibody may be readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence. The vector is typically transformed into a host cell suitable for expression of the nucleic acid. In some embodiments, the host cell is a eukaryotic cell or a prokaryotic cell. In some embodiments, the eukaryotic host cell is a mammalian cell. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J., 2003), pp. 255-268. The anti-CD38 antibody prepared from the cells can be purified using, for example, hydroxylapatite chromatography, hydrophobic interaction chromatography, gel electrophoresis, dialysis, and affinity chromatography, with affinity chromatography being among one of the typically preferred purification steps. In general, various methodologies for preparing antibodies for use in research, testing, and clinical applications are well-established in the art, consistent with the above-described methodologies and/or as deemed appropriate by one skilled in the art.

### Methods of Treatment

Treatment Comprising Intravenous Infusion of Anti-CD38 Antibody from a Fixed Volume of 250 ml

Provided herein are methods of administering (e.g., safely administering) an anti-CD38 antibody to in an individual (e.g., a human individual) in need thereof, comprising administering to the individual a dose of 10 mg/kg (e.g., at least 10 mg/kg) of an anti-CD38 antibody (e.g., an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6)) via intravenous infusion, wherein each dose of the anti-CD38 antibody is in a volume of 250 ml. In some embodiments, the anti-CD38 antibody is isatuximab. In some embodiments, the individual does not experience an infusion reaction (IR) during or following the administration of the anti-CD38 antibody via intravenous infusion, wherein the anti-CD38 antibody is in a volume of 250 ml. In some embodiments, the individual experiences only mild IR during or following the administration of the anti-CD38 antibody via intravenous infusion, wherein the anti-CD38 antibody is in a volume of 250 ml. (Further details regarding IRs and characteristics of mild IRs are provided elsewhere herein.)

Provided herein are methods for treating or delaying progression of multiple myeloma (such as relapsed multiple myeloma or relapsed and refractory multiple myeloma) in an individual (e.g., a human individual) comprising administering to the individual 10 mg/kg (e.g., at least 10 mg/kg) of an anti-CD38 antibody (e.g., an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6)) via intravenous infusion, wherein each infusion is in a volume of 250 ml. In some embodiments, the anti-CD38 antibody is isatuximab. In some embodiments, the dose of anti-CD38 antibody (e.g., isatuximab) is 20 mg/kg.

In some embodiments, the anti-CD38 antibody (e.g., isatuximab) is administered to the individual in a first 28-day cycle. In some embodiments, the anti-CD38 antibody (e.g., isatuximab) is administered to the individual at a dose of 10 mg/kg (e.g., at least 10 mg/kg, or 20 mg/kg) in a volume of 250 ml on each of Days 1, 8, 15, and 22 of the first 28-day cycle. In some embodiments, the anti-CD38 antibody (e.g., isatuximab) is administered to the individual via intravenous infusion on Day 1 of the first 28 day cycle at an infusion rate of 25 mL/hour for a first hour, and the infusion rate is increased by 25 mL/hour every 30 minutes after the first hour to a maximum infusion rate of 150 mL/hour until the 250 ml of the anti-CD38 antibody (e.g., isatuximab) is infused. In some embodiments, the anti-CD38 antibody (e.g., isatuximab) is administered to the individual via intravenous infusion on Day 1 of the first 28 day cycle at an infusion rate of 12.5 mL/hour for a first 30 minutes, wherein the infusion rate is increased by 25 mL/hour every 30 minutes after the first 30 minutes until the 250 ml of the anti-CD38 antibody (e.g., isatuximab) is infused. In some embodiments, the duration of the infusion of the anti-CD38 antibody (e.g., isatuximab) on Day 1 of the first 28-day cycle is no more than any one of about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8. 4.9, 5.0, 5.1, 5.2, 5.3, 5.4, 5.5, 5.6, 5.7, 5.8, 5.9, 6.0, 6.2, 6.3, 6.4, or 6.5 hours, including any range in between these values. In some embodiments, the duration of the infusion of the anti-CD38 antibody (e.g., isatuximab) on Day 1 of the first 28-day cycle is between about 3.3 and about 6.1 hours, including any value within in this range. In some embodiments, the duration of the infusion of the anti-CD38 antibody (e.g., isatuximab) on Day 1 of the first 28-day cycle is between about 3.2 and 5.5 hours, such as between about 3.36 and about 5.32 hours. In some embodiments, the duration of the infusion of the anti-CD38 antibody (e.g., isatuximab) on Day 1 of the first 28-day cycle is between about 3.8 and 4.2 hours, such as about 3.94 hours. In some embodiments, the duration of infusion includes temporary interruptions prior to completion of the infusion.

In some embodiments, the anti-CD38 antibody (e.g., isatuximab) is administered to the individual via intravenous infusion on Day 8 of the first 28 day cycle at an infusion rate of 50 mL/hour for a first 30 minutes, wherein the infusion rate is increased by 50 ml/hr for the next 30 minutes, and wherein the infusion rate is increased by 100 mL/hour every 30 minutes after the first 60 minutes to a maximum infusion rate of 200 mL/hour until the 250 ml volume is infused. In some embodiments, the anti-CD38 antibody (e.g., isatuximab) is administered to the individual via intravenous infusion on Day 8 of the first 28 day cycle at an infusion rate of 50 mL/hour for a first 30 minutes, 100 mL/hour for a second 30 minutes, 200 mL for the third 30 minutes, and 300 mL/hour after the third 30 minutes until the 250 ml of the anti-CD38 antibody (e.g., isatuximab) is infused. In some embodiments, the anti-CD38 antibody (e.g., isatuximab) is administered to the individual via intravenous infusion on Day 8 of the first 28 day cycle at an infusion rate of 25 mL/hour for a first 30 minutes, and the infusion rate is increased by 50 mL/hour every 30 minutes after the first 30 minutes until the 250 ml of the anti-CD38 antibody (e.g., isatuximab) is infused. In some embodiments, the duration of the infusion of the anti-CD38 antibody (e.g., isatuximab) on Day 1 of the first 28-day cycle is no more than any one of about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 hours, including any range in between these values. In some embodiments, the duration of the infusion of the anti-CD38 antibody (e.g., isatuximab) on Day 8 of the first 28-day cycle is between about 1.5 and about 3.5 hours, including any value within in this range. In some embodiments, the duration of the infusion of the anti-CD38 antibody (e.g., isatuximab) on Day 8 of the first 28-day cycle is between about 1.4 and 2.7 hours, such as between about 1.52 and about 2.6 hours. In some embodiments, the duration of the infusion of the anti-CD38 antibody (e.g., isatuximab) on Day 8 of the first 28-day cycle is between about 1.5 and 2.0 hours, such as about 1.88 hours. In some embodiments, the duration of infusion of the anti-CD38 antibody (e.g., isatuximab) includes temporary interruptions prior to completion of the infusion.

In some embodiments, the anti-CD38 antibody (e.g., isatuximab) is administered to the individual via intravenous infusion on Day 15 of the first 28-day cycle at an infusion rate of 200 ml/hour until the 250 ml of the anti-CD38 antibody (e.g., isatuximab) is infused. In some embodiments, the anti-CD38 antibody (e.g., isatuximab) is administered to the individual via intravenous infusion on Day 15 of the first 28 day cycle at an infusion rate of 100 ml/hour for a first 30 minutes, and the infusion rate is increased by 50 mL/hour every 30 minutes after the first 30 minutes until the 250 ml of the anti-CD38 antibody (e.g., isatuximab) is infused. In some embodiments, the duration of the infusion of the anti-CD38 antibody (e.g., isatuximab) on Day 15 of the first 28-day cycle is no more than any one of about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 hours, including any range in between these values. In some embodiments, the duration of the infusion of the anti-CD38 antibody (e.g., isatuximab) on Day 1 of the first 28-day cycle is between about 1.2 and about 3.4 hours, including any value within in this range. In some embodiments, the duration of the infusion the anti-CD38 antibody (e.g., isatuximab) on Day 15 of the first 28-day cycle is between about 1 and 2 hours, such as between about 1.03 and about 1.87 hours. In some embodiments, the duration of the infusion on Day 15 of the first 28-day cycle is between about 1 and 1.5 hours, such as about 1.27 hours. In some embodiments, the duration of infusion of the anti-CD38 antibody (e.g., isatuximab) includes temporary interruptions prior to completion of the infusion.

In some embodiments, the anti-CD38 antibody (e.g., isatuximab) is administered to the individual via intravenous infusion on Day 22 of the first 28-day cycle at an infusion rate of 200 ml/hour until the 250 ml of the anti-CD38 antibody (e.g., isatuximab) is infused. In some embodiments, the anti-CD38 antibody (e.g., isatuximab) is administered to the individual via intravenous infusion on Day 22 of the first 28 day cycle at an infusion rate of 100 ml/hour for a first 30 minutes, and wherein the infusion rate is increased by 50 mL/hour every 30 minutes after the first 30 minutes until the 250 ml of the anti-CD38 antibody (e.g., isatuximab) is infused. In some embodiments, the duration of the infusion of the anti-CD38 antibody (e.g., isatuximab) on Day 22 of the first 28-day cycle is no more than any one of about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 hours, including any range in between these values. In some embodiments, the duration of the infusion of the anti-CD38 antibody (e.g., isatuximab) on Day 22 of the first 28-day cycle is between about 1.1 and about 2 hours, including any value within in this range. In some embodiments, the duration of the infusion of the anti-CD38 antibody (e.g., isatuximab) on Day 22 of the first 28-day cycle is between about 1 and 2 hours, such as between about 1.18 and about 1.52 hours. In some embodiments, the duration of the infusion of the anti-CD38 antibody (e.g., isatuximab) on Day 22 of the first 28-day cycle is between about 1 and 1.5 hours, such as about 1.27 hours. In some embodiments, the duration of infusion of the anti-CD38 antibody (e.g., isatuximab) includes temporary interruptions prior to completion of the infusion.

In some embodiments, the anti-CD38 antibody (e.g., isatuximab) is further administered via intravenous infusion in one or more subsequent 28-day cycles (e.g., following the first 28-day cycle) at a dose of 10 mg/kg (e.g., at least 10 mg/kg, or 20 mg/kg) on each of Days 1 and 15 of each subsequent 28-day cycle, wherein the anti-CD38 antibody is in a volume of 250 ml. In some embodiments, the anti-CD38 antibody (e.g., isatuximab) is administered to the individual via intravenous infusion on Day 1 of each subsequent 28-day cycle (e.g., following the first 28-day cycle) at an infusion rate of 200 ml/hour until the 250 ml of the anti-CD38 antibody (e.g., isatuximab) is infused. In some embodiments, the anti-CD38 antibody (e.g., isatuximab) is administered to the individual via intravenous infusion on Day 1 of each subsequent 28 day cycle (e.g., following the first 28-day cycle) at an infusion rate of 100 ml/hour for a first 30 minutes, and wherein the infusion rate is increased by 50 mL/hour every 30 minutes after the first 30 minutes until the 250 ml of the anti-CD38 antibody (e.g., isatuximab) is infused. In some embodiments, the duration of the infusion of the anti-CD38 antibody (e.g., isatuximab) on Day 1 of each subsequent 28 day cycle (e.g., following the first 28-day cycle) is no more than any one of about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 hours, including any range in between these values. In some embodiments, the duration of the infusion of the anti-CD38 antibody (e.g., isatuximab) on Day 1 of each subsequent 28-day cycle (e.g., following the first 28-day cycle) is between about 1.1 and about 1.6 hours, including any value within in this range. In some embodiments, the duration of the infusion of the anti-CD38 antibody (e.g., isatuximab) on Day 1 of each subsequent 28-day cycle (e.g., following the first 28-day cycle) is between about 1 and 2 hours, such as between about 1.19 and about 1.41 hours. In some embodiments, the duration of the infusion of the anti-CD38 antibody (e.g., isatuximab) on Day 1 of each subsequent 28-day cycle (e.g., following the first 28-day cycle) is between about 1 and 1.5 hours, such as about 1.27 hours. In some embodiments, the duration of infusion of the anti-CD38 antibody (e.g., isatuximab) includes temporary interruptions prior to completion of the infusion. In some embodiments, the anti-CD38 antibody (e.g., isatuximab) is administered to the individual via intravenous infusion on Day 15 of each subsequent 28-day cycle (e.g., following the first 28-day cycle) at an infusion rate of 200 ml/hour until the 250 ml of the anti-CD38 antibody (e.g., isatuximab) is infused. In some embodiments, the anti-CD38 antibody (e.g., isatuximab) is administered to the individual via intravenous infusion on Day 15 of each subsequent 28 day cycle (e.g., following the first 28 day cycle) at an infusion rate of 100 ml/hour for a first 30 minutes, and wherein the infusion rate is increased by 50 mL/hour every 30 minutes after the first 30 minutes until the 250 ml of the anti-CD38 antibody (e.g., isatuximab) is infused. In some embodiments, the duration of the infusion of the anti-CD38 antibody (e.g., isatuximab) on Day 15 of each subsequent 28 day cycle (e.g., following the first 28-day cycle) is no more than any one of about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 hours, including any range in between these values. In some embodiments, the duration of the infusion of the anti-CD38 antibody (e.g., isatuximab) on Day 1 of each subsequent 28-day cycle (e.g., following the first 28-day cycle) is between about 1.2 and about 1.6 hours, including any value within in this range. In some embodiments, the duration of the infusion of the anti-CD38 antibody (e.g., isatuximab) on Day 1 of each subsequent 28-day cycle (e.g., following the first 28-day cycle) is between about 1 and 2 hours, such as between about 1.2 and about 1.46 hours. In some embodiments, the duration of the infusion of the anti-CD38 antibody (e.g., isatuximab) on Day 1 of each subsequent 28-day cycle (e.g., following the first 28-day cycle) is between about 1 and 1.5 hours, such as about 1.27 hours. In some embodiments, the duration of infusion of the anti-CD38 antibody (e.g., isatuximab) includes temporary interruptions prior to completion of the infusion.

In some embodiments, the duration of each infusion of the anti-CD38 antibody (e.g., isatuximab) on or after Day 15 of the first 28 day cycle (e.g., including Day 22 of the first 28 day cycle and Day 1 and Day 15 of each subsequent 28-day cycle) is no more than any one of about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 hours, including any range in between these values. In some embodiments, the duration of each infusion of the anti-CD38 antibody (e.g., isatuximab) on or after Day 15 of the first 28-day cycle (e.g., including Day 22 of the first 28-day cycle and Day 1 and Day 15 of each subsequent 28-day cycle) is between about 0.7 and about 3.4 hours, including any value within in this range. In some embodiments, the duration of each infusion of the anti-CD38 antibody (e.g., isatuximab) on or after Day 15 of the first 28 day cycle (e.g., including Day 22 of the first 28 day cycle and Day 1 and Day 15 of each subsequent 28-day cycle) is between about 1 and 2 hours, such as between about 1.13 and about 1.53 hours. In some embodiments, the duration of the infusion of the anti-CD38 antibody (e.g., isatuximab) on Day 1 of each subsequent 28-day cycle (e.g., following the first 28-day cycle) is between about 1 and 1.5 hours, such as about 1.25 hours.

In some embodiments, the duration of each infusion of the anti-CD38 antibody (e.g., isatuximab) after the first infusion (e.g., on Day 1 of the first 28-day cycle) is no more than 0.5 hours. In some embodiments, the duration of each infusion of the anti-CD38 antibody (e.g., isatuximab) after Day 1 of the first 28 day cycle (e.g., including Days 8, 15 and 22 of the first 28 day cycle and Day 1 and Day 15 of each subsequent 28-day cycle) is no more than any one of about 0.5 hours. In some embodiments, the duration of each infusion of the anti-CD38 antibody (e.g., isatuximab) on or after Day 8 of the first 28 day cycle (e.g., including Days 15 or 22 of the first 28 day cycle and Day 1 and Day 15 of each subsequent 28-day cycle) is no more than any one of about 0.5 hours.

Also provided herein is a method of safely administering an anti-CD38 antibody to a human individual in need thereof, comprising administering at least a first 10 mg/kg dose (e.g., at least 10 mg/kg, or 20 mg/kg) of an anti-CD38 antibody via intravenous infusion (i.e., a first intravenous infusion), wherein the anti-CD38 antibody is in a volume of 250 ml, and wherein the anti-CD38 antibody comprises (a) a heavy chain variable domain (VH) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (VL) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6). In some embodiments, the anti-CD38 antibody is isatuximab. In some embodiments of safely administering the anti-CD38 antibody (e.g., isatuximab), the individual does not experience grade 3 or higher IR during or after the infusion of the anti-CD38 antibody. In some embodiments of safely administering the anti-CD38 antibody (e.g., isatuximab), the individual does not experience Grade 2 or higher IR during or after the second or subsequence infusion of the anti-CD38 antibody.

In some embodiments, the first intravenous infusion of a 10 mg/kg dose (i.e., first dose of, e.g., at least a 10 mg/kg dose, or a 20 mg/kg dose) of the anti-CD38 antibody (e.g., isatuximab) is in a volume of 250 ml and is administered to the individual at an infusion rate of 25 mL/hour for a first hour, wherein the infusion rate is increased by 25 mL/hour every 30 minutes after the first hour to a maximum infusion rate of 150 mL/hour until the 250 ml of the anti-CD38 antibody (e.g., isatuximab) is infused. In some embodiments, the first intravenous infusion of a 10 mg/kg dose (e.g., at least a 10 mg/kg dose, or a 20 mg/kg dose) of the anti-CD38 antibody (e.g., isatuximab) is in a volume of 250 ml and is administered to the individual at an infusion rate of 12.5 mL/hour for a first 30 minutes, and wherein the infusion rate is increased by 25 mL/hour every 30 minutes after the first 30 minutes until the 250 ml of the anti-CD38 antibody (e.g., isatuximab) is infused. In some embodiments, the duration of the first intravenous infusion of a 10 mg/kg dose of the anti-CD38 antibody (e.g., isatuximab) is no more than is no more than any one of about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8. 4.9, 5.0, 5.1, 5.2, 5.3, 5.4, 5.5, 5.6, 5.7, 5.8, 5.9, 6.0, 6.2, 6.3, 6.4, or 6.5 hours, including any range in between these values. In some embodiments, the duration of the first intravenous infusion of a 10 mg/kg dose (e.g., at least a 10 mg/kg dose, or a 20 mg/kg dose) of the anti-CD38 antibody (e.g., isatuximab) is between about 3.3 and about 6.1 hours, including any value within in this range. In some embodiments, the duration of the first intravenous infusion of a 10 mg/kg dose (e.g., at least a 10 mg/kg dose, or a 20 mg/kg dose) of the anti-CD38 antibody (e.g., isatuximab) is between about 3.2 and 5.5 hours, such as between about 3.36 and about 5.32 hours. In some embodiments, the duration of the first intravenous infusion of a 10 mg/kg dose (e.g., at least a 10 mg/kg dose, or a 20 mg/kg dose) of the anti-CD38 antibody (e.g., isatuximab) is between about 3.8 and 4.2 hours, such as about 3.94 hours. In some embodiments, the duration of the first infusion of the anti-CD38 antibody (e.g., isatuximab) includes temporary interruptions prior to completion of the infusion.

In some embodiments, a second 10 mg/kg dose (e.g., at least a 10 mg/kg dose, or a 20 mg/kg dose) of the anti-CD38 antibody (e.g., isatuximab) is administered to the individual in need thereof via intravenous infusion (i.e., a second intravenous infusion), wherein the anti-CD38 antibody is in a volume of 250 ml.

In some embodiments, the second intravenous infusion of a 10 mg/kg dose (i.e., second dose of, e.g., at least a 10 mg/kg dose, or a 20 mg/kg dose) of the anti-CD38 antibody (e.g., isatuximab) is in a volume of 250 ml and is administered to the individual at an infusion rate of 50 mL/hour for a first 30 minutes, wherein the infusion rate is increased by 50 ml/hr for a second 30 minutes, and wherein the infusion rate is increased by 100 mL/hour every 30 minutes after the second 30 minutes to a maximum infusion rate of 200 mL/hour until the 250 ml volume is infused. In some embodiments, the second intravenous infusion of a 10 mg/kg dose (i.e., second dose of, e.g., at least a 10 mg/kg dose, or a 20 mg/kg dose) of the anti-CD38 antibody (e.g., isatuximab) is in a volume of 250 ml and is administered to the individual at an infusion rate of 50 mL/hour for a first 30 minutes, 100 mL/hour for a second 30 minutes, 200 mL for the third 30 minutes, and 300 mL/hour after the third 30 minutes until the 250 ml of the anti-CD38 antibody (e.g., isatuximab) is infused. In some embodiments, the second intravenous infusion of a 10 mg/kg dose (e.g., at least a 10 mg/kg dose, or a 20 mg/kg dose) of the anti-CD38 antibody (e.g., isatuximab) is in a volume of 250 ml and is administered to the individual at an infusion rate of 25 mL/hour for a first 30 minutes, and wherein the infusion rate is increased by 50 mL/hour every 30 minutes after the first 30 minutes until the 250 ml of the anti-CD38 antibody (e.g., isatuximab) is infused. In some embodiments, the duration of the second infusion of a 10 mg/kg dose of the anti-CD38 antibody (e.g., isatuximab) is no more than is no more than any one of about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 hours, including any range in between these values. In some embodiments, the duration of the second infusion of a 10 mg/kg dose (e.g., at least a 10 mg/kg dose, or a 20 mg/kg dose) of the anti-CD38 antibody (e.g., isatuximab) is between about 1.5 and about 3.5 hours, including any value within in this range. In some embodiments, the duration of the second infusion of a 10 mg/kg dose (e.g., at least a 10 mg/kg dose, or a 20 mg/kg dose) of the anti-CD38 antibody (e.g., isatuximab) is between about 1.4 and 2.7 hours, such as between about 1.52 and about 2.6 hours. In some embodiments, the duration of the second infusion of a 10 mg/kg dose (e.g., at least a 10 mg/kg dose, a 20 mg/kg dose) of the anti-CD38 antibody (e.g., isatuximab) is about between about 1.5 and 2.0 hours, such as about 1.88 hours. In some embodiments, the duration of the second infusion of the anti-CD38 antibody (e.g., isatuximab) includes temporary interruptions prior to completion of the infusion.

In some embodiments, a third 10 mg/kg dose (e.g., at least a 10 mg/kg dose, or a 20 mg/kg dose) of the anti-CD38 antibody (e.g., isatuximab) is in a 250 ml volume and is administered to the individual in need thereof via intravenous infusion (i.e., a third intravenous infusion. In some embodiments, the third infusion of a 10 mg/kg dose (i.e., a third dose of, e.g., at least a 10 mg/kg dose, or a 20 mg/kg dose) of the anti-CD38 antibody (e.g., isatuximab) is in a volume of 250 ml and is administered to the individual at an infusion rate of 200 ml/hour until the 250 ml of the anti-CD38 antibody (e.g., isatuximab) is infused. In some embodiments, the third infusion of a 10 mg/kg dose (e.g., at least a 10 mg/kg dose, or a 20 mg/kg dose) of anti-CD38 antibody (e.g., isatuximab) is in a volume of 250 ml and is administered to the individual at an infusion rate of 100 ml/hour for a first 30 minutes, and wherein the infusion rate is increased by 50 mL/hour every 30 minutes after the first 30 minutes until the 250 ml of the anti-CD38 antibody (e.g., isatuximab) is infused. In some embodiments, the duration of the third infusion of a 10 mg/kg dose (e.g., at least a 10 mg/kg dose, or a 20 mg/kg dose) of the anti-CD38 antibody (e.g., isatuximab) is no more than any one of about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 hours, including any range in between these values. In some embodiments, the duration of the infusion of the third 10 mg/kg dose (e.g., at least a 10 mg/kg dose, or a 20 mg/kg dose) of the anti-CD38 antibody (e.g., isatuximab) is between about 1.2 and about 3.4 hours, including any value within in this range. In some embodiments, the duration of the infusion the third 10 mg/kg dose (e.g., at least a 10 mg/kg dose, or a 20 mg/kg dose) of the anti-CD38 antibody (e.g., isatuximab) is between about 1 and 2 hours, such as between about 1.03 and about 1.87 hours. In some embodiments, the duration of the infusion the third 10 mg/kg dose (e.g., at least a 10 mg/kg dose, or a 20 mg/kg dose) of the anti-CD38 antibody (e.g., isatuximab) is between about 1 and 1.5 hours, such as about 1.27 hours. In some embodiments, the duration of the third infusion of 10 mg/kg dose (e.g., at least a 10 mg/kg dose, or a 20 mg/kg dose) the anti-CD38 antibody (e.g., isatuximab) includes temporary interruptions prior to completion of the infusion.

In some embodiments, one or more subsequent intravenous infusions of the anti-CD38 antibody (e.g., isatuximab) are administered to the individual following the third intravenous infusion, wherein each of the one or more subsequent infusions provides a 10 mg/kg dose (e.g., at least 10 mg/kg, or 20 mg/kg), e.g., fourth dose, fifth dose, sixth dose, etc., of the anti-CD38 antibody (e.g., isatuximab) to the individual in need thereof, and wherein each of the one or more subsequent infusions of the anti-CD38 antibody is in a volume of 250 ml. The one or more subsequent infusions include, but are not limited to, e.g., a fourth infusion, a fifth infusion, a sixth infusion, etc. In some embodiments, the one or more subsequent infusions of the anti-CD38 antibody (e.g., isatuximab) are each in a volume of 250 ml and are each administered to the individual at an infusion rate of 200 ml/hour until the 250 ml of the anti-CD38 antibody (e.g., isatuximab) is infused. In some embodiments, the one or more subsequent infusions of the anti-CD38 antibody (e.g., isatuximab) are each administered to the individual at an infusion rate of 100 ml/hour for a first 30 minutes, and wherein the infusion rate is increased by 50 mL/hour every 30 minutes after the first 30 minutes until the 250 ml of the anti-CD38 antibody (e.g., isatuximab) is infused. In some embodiments, the duration of each of the one or more subsequent infusions of the anti-CD38 antibody (e.g., isatuximab) is no more than is no more than any one of about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 hours, including any range in between these values. In some embodiments, the duration of each of the one or more subsequent infusions of the anti-CD38 antibody (e.g., isatuximab) is between about 0.7 and about 3.4 hours, such as between about 1.1 and about 1.6 hours, including any value within in these ranges. In some embodiments, the duration of each of the one or more subsequent infusions of the anti-CD38 antibody (e.g., isatuximab) is between about 1 and 2 hours, such as between about 1.13 and about 1.53, or between about 1.19 and about 1.41 hours, including any value within these ranges. In some embodiments, the duration of each of the one or more subsequent infusions of the anti-CD38 antibody (e.g., isatuximab) is about between about 1 and 1.5 hours, such as about 1.27 hours or 1.25 hours. In some embodiments, the duration of each of the one or more subsequent infusions of the anti-CD38 antibody (e.g., isatuximab) includes temporary interruptions prior to completion of the infusion.

In some embodiments, the duration of each infusion that provides a 10 mg/kg dose (e.g., at least a 10 mg/kg dose, or 20 mg/kg dose) of the anti-CD38 antibody (e.g., isatuximab) after the first infusion (e.g., on Day 1 of the first 28-day cycle) is no more than 0.5 hours. In some embodiments, the duration of each infusion that provides a 10 mg/kg dose (e.g., at least a 10 mg/kg dose, or 20 mg/kg dose) of the anti-CD38 antibody (e.g., isatuximab) after Day 1 of the first 28 day cycle (e.g., including Days 8, 15 and 22 of the first 28 day cycle and Day 1 and Day 15 of each subsequent 28-day cycle) is no more than about 0.5 hours. In some embodiments, the duration of each infusion that provides a 10 mg/kg dose (e.g., at least a 10 mg/kg dose, or 20 mg/kg dose) of the anti-CD38 antibody (e.g., isatuximab) on or after Day 8 of the first 28 day cycle (e.g., including Days 15 or 22 of the first 28 day cycle and Day 1 and Day 15 of each subsequent 28-day cycle) is no more than about 0.5 hours.

In some embodiments, the individual does not experience an infusion reaction (IR) during or following administration (e.g., intravenous infusion) of the anti-CD38 antibody (such as isatuximab) at a dose of 10 mg/kg (e.g., at least 10 mg/kg, or 20 mg/kg) in a 250 ml volume. In some embodiments, administration of the anti-CD38 antibody (e.g., via intravenous infusion) at a dose of 10 mg/kg (e.g., at least 10 mg/kg, or 20 mg/kg) in a 250 ml volume does not cause the individual to experience an IR during or following administration. In some embodiments, the individual does not experience an IR of Grade 3 or higher during or following the infusion of the anti-CD38 antibody (e.g., isatuximab). In some embodiments, the individual does not experience IR during or following the second infusion of the anti-CD38 antibody (e.g., isatuximab). In some embodiments, the individual does not experience an IR during or following the second infusion of the anti-CD38 antibody (e.g., isatuximab) or in subsequent infusions of the anti-CD38 antibody (e.g., isatuximab). An IR refers to a disorder characterized by adverse reaction to the intravenous infusion of an anti-CD38 antibody (e.g., isatuximab). An IR may occur during the fusion or within 24 hours of the infusion (such as 24 hours from the time the infusion started). Signs or symptoms of an IR include one or more of the following: paresthesia, chest pain, cough, nasal congestion, sneezing, throat irritation, pruritus, syncope, flushing, chills, fever, urticarial, angioedema, rash, skin reactions, itching, maculopapular rash, tachycardia, hypotension, dyspnea, nausea, vomiting, headache, back pain, chest discomfort or non-cardiac chest pain, abdominal pain, abdominal cramps, bronchospasm, laryngospasm, wheezing, respiratory tract congestion, excessive sweating, and erythema. (See, e.g., Doessegger et al. (2015) Clin & Trans Immunol. 4(7): e39 for further details.) Thus, in some embodiments, the individual does not experience any one or more of these signs or symptoms.

In some embodiments, the individual receives (e.g., requires) premedication, i.e., medication administered prior the infusion of the anti-CD38 antibody (e.g. isatuximab) for the purpose of preventing or minimizing an IR. In some embodiments, the individual receives premedication with one or more of: an analgesic (e.g., acetaminophen or paracetamol), an H2 antagonist or antacid (such as ranitidine, cimetidine, omeprazole, or esomeprazole), an anti-inflammatory agent (such as a corticosteroid or a nonsteroidal anti-inflammatory drug), and/or an antihistamine (such as diphenhydramine, cetirizine, promethazine, dexchlorpheniramine) for the purpose of preventing or minimizing an IR prior to infusion of the anti-CD38 antibody (such as isatuximab) at a dose of 10 mg/kg (e.g., at least 10 mg/kg, or 20 mg/kg) in a 250 ml volume.

In some embodiments, the individual does not receive (e.g., require) premedication, i.e., medication administered prior the infusion of the anti-CD38 antibody (e.g. isatuximab) for the purpose of preventing or minimizing an IR. In some embodiments, the individual does not receive (e.g., require) premedication with one or more of: an analgesic (e.g., acetaminophen or paracetamol), an H2 antagonist or antacid (such as ranitidine, cimetidine, omeprazole, or esomeprazole), an anti-inflammatory agent (such as a corticosteroid or a nonsteroidal anti-inflammatory drug), and/or an antihistamine (such as diphenhydramine, cetirizine, promethazine, dexchlorpheniramine) for the purpose of preventing or minimizing an IR prior to infusion of the anti-CD38 antibody (such as isatuximab) at a dose of 10 mg/kg (e.g., at least 10 mg/kg, or 20 mg/kg) in a 250 ml volume. In some embodiments, the individual does not receive (e.g., require) medication (e.g., prophylactic medication) to prevent or minimize an IR following completion of the infusion of the anti-CD38 antibody (e.g., isatuximab). In some embodiments, the individual does not experience a delayed infusion reaction following administration (e.g., intravenous infusion) of the anti-CD38 antibody (such as isatuximab) at a dose of 10 mg/kg (e.g., at least 10 mg/kg, or 20 mg/kg) in a 250 ml volume. In some embodiments, the individual does not experience a delayed infusion reaction within about any one of 0.5, 1.0, 1.5, 2.0, 2.5, 3.0, 6.0, 9.0, 12.0, 18.0, 21.0, or 24.0 hours (including any range in between these values) following administration (e.g., intravenous infusion) of the anti-CD38 antibody (such as isatuximab) at a dose of 10 mg/kg (e.g., at least 10 mg/kg, or 20 mg/kg) in a 250 ml volume. In some embodiments, the individual does not receive (e.g., require) premedication or prophylactic medication, e.g., as described above, prior to the first, second, third, fourth, and/or fifth infusions in a 250 ml volume. In some embodiments, the individual does not receive (e.g., require) premedication or prophylactic medication, e.g., as described above, prior to the first, second, third, and/or fourth infusions of a dose of 10 mg/kg (e.g., at least 10 mg/kg, or 20 mg/kg) anti-CD38 antibody in a 250 ml volume. In some embodiments, the individual does not receive (e.g., require) premedication or prophylactic medication, e.g., as described above, prior to start of the fourth infusion of a dose of 10 mg/kg (e.g., at least 10 mg/kg, or 20 mg/kg) anti-CD38 antibody in a 250 ml volume. In some embodiments, the individual does not receive (e.g., require) premedication or prophylactic medication, e.g., as described above, prior the start of any infusion after the third infusion of a dose of 10 mg/kg (e.g., at least 10 mg/kg, or 20 mg/kg) anti-CD38 antibody in a 250 ml volume. In some embodiments, the individual does not receive (e.g., require) premedication or prophylactic medication, e.g., as described above, prior to any infusion of a dose of 10 mg/kg (e.g., at least 10 mg/kg, or 20 mg/kg) anti-CD38 antibody in a 250 ml volume. In some embodiments, the individual does not receive (e.g., require) post-medication, i.e., medication administered following completion of the infusion (e.g., within at least about any one of 0.5, 1.0, 1.5, 2.0, 2.5, or 3.0 hours of completion of the infusion, including any range between these values) of the anti-CD38 antibody (e.g. isatuximab) at a dose of 10 mg/kg (e.g., at least 10 mg/kg, or 20 mg/kg) in a 250 ml volume for the purpose of preventing or minimizing an IR. In some embodiments, the individual does not receive (e.g., require) post-medication within at least about any one of 0.5, 1.0, 1.5, 2.0, 2.5, 3.0, 6.0, 9.0, 12.0, 18.0, 21.0, or 24.0 hours (including any range in between these values) following the completion of an infusion of the anti-CD38 antibody (e.g. isatuximab) at a dose of 10 mg/kg in a 250 ml volume, e.g., for the purpose of preventing or minimizing an IR. In some embodiments, the individual does not receive (e.g., require) post-medication, e.g., as described above, following the completion (e.g., within at least about any one of about any one of 0.5, 1.0, 1.5, 2.0, 2.5, 3.0, 6.0, 9.0, 12.0, 18.0, 21.0, or 24.0 hours of completion, including any range in between these values) of the first, second, third, fourth, and/or fifth infusions of a dose of 10 mg/kg (e.g., at least 10 mg/kg, or 20 mg/kg) anti-CD38 antibody in a 250 ml volume. In some embodiments, the individual does not receive (e.g., require) post-medication, e.g., as described above, following the completion (e.g., within at least about any one of about any one of 0.5, 1.0, 1.5, 2.0, 2.5, 3.0, 6.0, 9.0, 12.0, 18.0, 21.0, or 24.0 hours of completion, including any range in between these values) of the first, second, third, and/or fourth infusions of a dose of 10 mg/kg (e.g., at least 10 mg/kg, or 20 mg/kg) anti-CD38 antibody in a 250 ml volume. In some embodiments, the individual does not receive (e.g., require) post-medication, e.g., as described above, following the completion (e.g., within at least about any one of about any one of 0.5, 1.0, 1.5, 2.0, 2.5, 3.0, 6.0, 9.0, 12.0, 18.0, 21.0, or 24.0 hours of completion, including any range in between these values) of the fourth infusion of a dose of 10 mg/kg (e.g., at least 10 mg/kg, or 20 mg/kg) anti-CD38 antibody in a 250 ml volume. In some embodiments, the individual does not receive (e.g., require) post-medication, e.g., as described above, following the completion (e.g., within at least about any one about any one of 0.5, 1.0, 1.5, 2.0, 2.5, 3.0, 6.0, 9.0, 12.0, 18.0, 21.0, or 24.0 hours if completion, including any range in between these values) of any infusion subsequent to the third infusion of a dose of 10 mg/kg (e.g., at least 10 mg/kg, or 20 mg/kg) anti-CD38 antibody in a 250 ml volume. In some embodiments, the individual does not receive (e.g., require) post-medication, e.g., as described above, following the completion (e.g., within at least about any one of about any one of 0.5, 1.0, 1.5, 2.0, 2.5, 3.0, 6.0, 9.0, 12.0, 18.0, 21.0, or 24.0 hours of completion, including any range in between these values) of any infusion of a dose of 10 mg/kg (e.g., at least 10 mg/kg, or 20 mg/kg) anti-CD38 antibody in a 250 ml volume. In some embodiments the individual does not receive premedication or post-medication with any one or more of: an analgesic (e.g., acetaminophen or paracetamol), an H2 antagonist or antacid (such as ranitidine, cimetidine, omeprazole, or esomeprazole), an anti-inflammatory agent (such as a corticosteroid or a nonsteroidal anti-inflammatory drug), and/or an antihistamine (such as diphenhydramine, cetirizine, promethazine, dexchlorpheniramine) for the purpose of preventing or minimizing an IR prior to infusion of the anti-CD38 antibody (such as isatuximab) at a dose of 10 mg/kg (e.g., at least 10 mg/kg, or 20 mg/kg) in a 250 ml volume.

In some embodiments, the individual experiences a mild IR following administration of the anti-CD38 antibody (such as isatuximab). In some embodiments, the mild IR is no more than a Grade 1 or Grade 2 IR, as defined in the National Cancer Institute Common Terminology Criteria for Adverse Events, version 4.03 (NCI-CTCAE v. 4.03). The NCI-CTCAE v. 4.03 is publicly available online at evs(dot)nci(dot)nih(dot)gov/ftp1/CTCAE/About(dot)html. In some embodiments, the IR is a Grade 1 IR if the individual experiences a mild transient reaction (e.g., one or more of the signs/symptoms described herein, such as within 24 hours of the start of the infusion), wherein the interruption of the infusion is not indicated and/or wherein intervention is not indicated. In some embodiment, the IR is a Grade 2 IR if the individual experiences a reaction (e.g., one or more of the signs/symptoms described herein, such as within 24 hours of the start of the infusion), wherein infusion is interrupted and/or wherein intervention is indicated, and wherein the individual responds promptly to treatment (i.e., treatment of the one or more signs or symptoms of IR, such as those described herein), such as within about any one of 2, 4, 6, 8, 10, 12, 14, 16, 18, 20, or 24 hours of the treatment for the IR (including any range between these values). In some embodiments, the treatment for the IR comprises one or more of: short-term interruption of the infusion, administration of oxygen, administration of bronchodilators, administration of corticosteroids, administration of histamine blockers, and restarting the infusion at a slower rate

In some embodiments, the individual experiences a mild IR (e.g., a Grade 1 or Grade 2 IR) during or following the first intravenous infusion of 10 mg/kg (e.g., at least 10 mg/kg, or 20 mg/kg) of the anti-CD38 antibody (such as isatuximab) in a 250 ml fixed volume, e.g., during infusion on Day 1 of the first 28-day cycle. In some embodiments, the individual experiences no IR (or no further IR) during a second or subsequent infusion of the anti-CD38 antibody (e.g., isatuximab) in a 250 ml fixed volume. For example, in some embodiments, the individual experiences no IR (or no further IR) during infusion of 10 mg/kg (e.g., at least 10 mg/kg, or 20 mg/kg) of the anti-CD38 antibody (such as isatuximab) in a 250 ml fixed volume on any of Days 8, 15, and 22 of the first 28-day cycle and on any of Days 1 and 15 of any subsequent 28-day cycle.

In some embodiments, the individual does not experience a moderate or severe IR following infusion of an anti-CD38 antibody in a 250 ml volume, e.g., according to a method described herein. In some embodiments, the individual does not experience an IR of Grade 3, 4, or 5, as defined in the National Cancer Institute Common Terminology Criteria for Adverse Events, version 4.03 (NCI-CTCAE v. 4.03). In some embodiments, the IR is a Grade 3 IR if the individual experiences prolonged signs/symptoms of IR (such as described herein) and is not rapidly responsive to medication for the IR and/or to interruption of the infusion. In some embodiments, the IR is Grade 3 IR if the individual experiences recurrence of the signs/symptoms of IR (such as described herein) following initial improvement. In some embodiments, the IR is grade 3 IR is the individual requires hospitalization for the signs/symptoms of IR (such as described herein). In some embodiments, the IR is a Grade 4 IR if the signs/symptoms (such as described herein) are life threatening and/or require urgent intervention. In some embodiments, the IR is Grade 5 IR if the signs/symptoms of IR result in death.

In some embodiments, the individual does not experience an IR of any grade (e.g., Grade 1, 2, 3, 4, or 5 IR) during or following the fourth intravenous infusion of 10 mg/kg (e.g., at least 10 mg/kg, or 20 mg/kg) of the anti-CD38 antibody (such as isatuximab) in a 250 ml fixed volume. Additionally or alternatively, in some embodiments, the individual does not experience an IR of any grade (e.g., Grade 1, 2, 3, 4, or 5 IR) during or following any intravenous infusion of 10 mg/kg (e.g., at least 10 mg/kg, or 20 mg/kg) of the anti-CD38 antibody (such as isatuximab) in a 250 ml fixed volume subsequent to the fourth intravenous infusion. In some embodiments, the individual experiences no IR (or no further IR) during a fourth infusion or an infusion after the fourth infusion of the anti-CD38 antibody (e.g., isatuximab) in a 250 ml fixed volume. For example, in some embodiments, the individual experiences no IR (or no further IR) during infusion of 10 mg/kg (e.g., at least 10 mg/kg, or 20 mg/kg) of the anti-CD38 antibody (such as isatuximab) in a 250 ml fixed volume on Day 22 of the first 28-day cycle and on any of Days 1 and 15 of any subsequent 28-day cycle (i.e., after the first 28-day cycle).

In some embodiments, the dose of anti-CD38 antibody (such as isatuximab) that is in a fixed 250 ml volume and is administered to the individual is not reduced during treatment, e.g., whether or not the individual experiences an IR.

In some embodiments, an anti-CD38 antibody described herein (such as isatuximab) is in a formulation comprising about 20 mg/mL antibody, about 20 mM histidine, about 10% (w/v) sucrose, about 0.02% (w/v) polysorbate 80 at pH 6.0. In some embodiments, an anti-CD38 antibody described herein (such as isatuximab) is in a formulation comprising about 20 mg/mL antibody, about 100 mg/mL sucrose, 2.22 mg/mL histidine hydrochloride monohydrate, about 1.46 mg/ml histidine, and about 0.2 mg/ml polysorbate 80. In some embodiments, the formulation comprises water for injection (WFI), such as sterile water for injection (SWFI). In some embodiments, the formulation is sterile. In some embodiments, a single use of the formulation comprises 5 ml of the formulation (i.e., 100 mg anti-CD38 antibody). In some embodiments, the single use 5 ml formulation is provided in, e.g., a type I 6 mL colorless clear glass vial fitted with elastomeric closure. In some embodiments, the fill volume of the vial has been established to ensure removal of 5 mL. In some embodiments, the fill volume is 5.4 mL. In some embodiments, a single use of the formulation comprises 25 ml of the formulation (i.e., 500 mg anti-CD38 antibody). In some embodiments, the single use 25 ml formulation is provided in, e.g., a 30 mL colorless clear glass vial fitted with elastomeric closure. In some embodiments, the fill volume of the vial has been established to ensure removal of 25 mL. In some embodiments, the formulation is stable for at least about 6, 12, 18, 24, 30, or 36 months, including any range in between these values, at a temperature between about 2° C. and about 8° C. and protected from light. In some embodiments, the formulation is diluted for infusion in 0.9% sodium chloride, 5% glucose, or 5% dextrose. In some embodiments, the diluted infusion solution is stable for up to about 6, 12, 18, 24, 30, 36, 42, or 48 hours, including any range in between these values, between about 2° C. and about 8° C. In some embodiments, the diluted solution for infusion is stable following storage (e.g., for up to about 6, 12, 18, 24, 30, 36, 42, or 48 hours, including any range in between these values) between about 2° C. and about 8° C. and for a further 8 hours (including the infusion time) at room temperature. In some embodiments, the diluted solution for infusion is stable in the presence of light. In some embodiments the bag in which the diluted solution for infusion is stored is fabricated from polyolefins (PO), polyethylene (PE), polypropylene (PP), polyvinyl chloride (PVC) with di(ethylhexyl)phthalate (DEHP) or ethy vinyl acetate (EVA). In some embodiments, the tubing used for infusion is fabricated from PE, PVC (with or without DEHP), polybutyldiene (PBD), or polyurethane (PU) with an in-line filter (polyethersulfone (PES), polysulfone or nylon).

For administration to patients, the appropriate volume of isatuximab is diluted in an infusion bag of 0.9% sodium chloride solution, 5% glucose, or 5% dextrose. No protection from light is required for storage in the infusion bags. The Investigational medicinal product was stored at +2° C. to +8° C.

In some embodiments, provided is an intravenous (IV) bag containing 250 mls of a 10 mg/kg dose (e.g., at least 10 mg/kg, or 20 mg/kg) of an anti-CD38 antibody (e.g., isatuximab). In some embodiments, the 10 mg/kg dose of the anti-CD38 antibody (e.g., isatuximab) is calculated based on the weight of the patient to whom the anti-CD38 antibody is to be administered. In some embodiments, the anti-CD38 antibody (e.g., isatuximab) is diluted from a concentrated formulation (e.g., a formulation described herein) into 0.9% sodium chloride, 5% glucose, or 5% dextrose. In some embodiments, the bag contains between about 360 mg and about 1600 mg, between about 450 mg and about 16000 mg, between about 450 mg and 1140 mg, or between about 450 mg and about 910 mg, including any range in between these values.

Treatment Comprising Administration of 10 mg/kg or 20 mg/kg Dose of Anti-CD38 Antibody

Also provided herein are methods for treating or delaying progression of multiple myeloma (such as relapsed multiple myeloma or relapsed and refractory multiple myeloma) in an individual (e.g., a human individual) comprising administering to the individual an anti-CD38 antibody (e.g., an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6) via intravenous infusion in a first 28 day cycle, and wherein the anti-CD38 antibody is administered at a dose of 10 mg/kg on Days 1, 8, 15, and 22 of the first 28 day cycle. In some embodiments, the anti-CD38 antibody is administered via intravenous infusion in one or more subsequent 28-day cycles following the first 28-day cycle, wherein the anti-CD38 antibody is administered at a dose of 10 mg/kg on Days 1 and 15 of each of the one or more subsequent 28-day cycles following the first 28-day cycle. In some embodiments, treatment results in a reduction of serum M protein by at least about any one of 40%, 45%, 50%, 55%, 60%, 65% or more than 65% from baseline. In some embodiments, treatment results in a reduction of serum M protein by at least about 52% from baseline. In some embodiments, serum M protein level is reduced after about two cycles of treatment. In some embodiments, the anti-CD38 antibody comprises a heavy chain variable region (V_{H}) comprising an amino acid sequence of SEQ ID NO: 7 and a light chain variable region (V_{L}) comprising an amino acid sequence of SEQ ID NO: 7 or SEQ ID NO: 9. In some embodiments, the anti-CD38 antibody is isatuximab. In some embodiments, the anti-CD38 antibody is not administered in combination with a second drug (i.e., the anti-CD38 antibody is administered as monotherapy).

Also provided herein are methods for treating or delaying progression of multiple myeloma (such as relapsed multiple myeloma or relapsed and refractory multiple myeloma) in an individual (e.g., a human individual) comprising administering to the individual an anti-CD38 antibody (e.g., an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6) via intravenous infusion in a first 28 day cycle, and wherein the anti-CD38 antibody is administered at a dose of 20 mg/kg on Days 1, 8, 15, and 22 of the first 28 day cycle. In some embodiments, the anti-CD38 antibody is administered via intravenous infusion in one or more subsequent 28-day cycles following the first 28-day cycle, wherein the anti-CD38 antibody is administered at a dose of 20 mg/kg on Days 1 and 15 of each of the one or more subsequent 28-day cycles following the first 28-day cycle. In some embodiments, treatment results in a reduction of serum M protein by at least about any one of 40%, 45%, 50%, 55%, 60%, 65% or more than 65% from baseline. In some embodiments, treatment results in a reduction of serum M protein by at least about 52% from baseline. In some embodiments, serum M protein level is reduced after about two cycles of treatment. In some embodiments, the anti-CD38 antibody comprises a heavy chain variable region (V_{H}) comprising an amino acid sequence of SEQ ID NO: 7 and a light chain variable region (V_{L}) comprising an amino acid sequence of SEQ ID NO: 7 or SEQ ID NO: 9. In some embodiments, the anti-CD38 antibody is isatuximab. In some embodiments, the anti-CD38 antibody is not administered in combination with a second drug (i.e., the anti-CD38 antibody is administered as monotherapy).

In some embodiments, the individual has received at least two, at least three, at least four, at least five, or at least six prior therapies (such as 7, 8, 9, 10, 11, or 12 prior therapies) for multiple myeloma. In some embodiments, the prior therapy for multiple myeloma was an immunomodulatory drug (e.g., lenalidomide, pomalidomide, and/or thalidomide). In some embodiments, the individual was refractory to the immunomodulatory drug. In some embodiments, the prior therapy for multiple myeloma was a proteasome inhibitor (e.g., bortezomib, carfilzomib, and/or ixazomib). In some embodiments, the individual was refractory to the proteasome inhibitor. In some embodiments, the individual received prior therapy with an immunomodulatory drug and a proteasome inhibitor. In some embodiments, the immunomodulatory drug and the proteasome inhibitor were administered in combination. In some embodiments, the immunomodulatory drug and the proteasome inhibitor were administered during separate therapies (e.g., separate treatment regimens). In some embodiments, the individual was refractory to the immunomodulatory drug and the proteasome inhibitor.

In some embodiments, the individual has at least one high-risk cytogenetic abnormality (e.g., prior to starting treatment with the anti-CD38 antibody). In some embodiments the at least one high-risk cytogenetic abnormality is selected from the group consisting of: 17p deletion/del(17p) (TP53), t(4;14) translocation (FGFR3/IGH), and t(14;16) translocation (IGH/MAF). In some embodiments, the individual has at least two high-risk cytogenetic abnormalities. In some embodiments, the individual has all three high-risk cytogenetic abnormalities.

### Other Characteristics of Individuals Receiving Treatment Comprising an Anti-CD38 Antibody

In some embodiments, the individual demonstrated progressive disease during the most recent prior therapy (or line of therapy), e.g., the therapy (or line of therapy) just before the start of a treatment described herein comprising administration of the anti-CD38 antibody (e.g., isatuximab). In some embodiments, the individual demonstrated progressive disease (PD) within 60 days after the end of the most recent prior therapy (or line of therapy) for multiple myeloma, e.g., the therapy (or line of therapy) just before the start of the treatment comprising administration of the anti-CD38 antibody (e.g., isatuximab) a treatment described herein comprising administration of the anti-CD38 antibody (e.g., isatuximab). In some embodiments, a progressive disease (PD) is defined according to International Myeloma Working Group criteria (see, e.g., Kumar et al. (2016) “International Myeloma Working Group consensus criteria for response and minimal residual disease assessment in multiple myeloma.” Lancet Oncol. 17(8):e328-e346; Durie et al. (2006) “International uniform response criteria for multiple myeloma. Leukemia. 20: 1467-1473; and Table 14 herein). In some embodiments, a line of therapy is ≥1 complete cycle of a single agent, or of a combination of two or more agents, or a planned sequential therapy that includes stem cell transplantation. In some embodiments, a given treatment is considered a new line of therapy if any 1 of the following 3 conditions are met:

1. Start of a new line of treatment after discontinuation of a previous line. If a treatment regimen is discontinued for any reason and a different regimen is started, it can be considered a new line of therapy. For example, a regimen is considered to have been discontinued if all the drugs in that given regimen have been stopped. For example, a regimen is not considered to have been discontinued if some of the drugs of the regimen, but not all, have been discontinued. In some embodiments, the reasons for discontinuation, addition, substitution, or SCT do not influence how lines are counted. Reasons for change may include, for example, end of planned therapy, toxicity, progression, lack of response, inadequate response.

2. The unplanned addition or substitution of 1 or more drugs in an existing regimen. Unplanned addition of a new drug or switching to a different drug (or combination of drugs) due to any reason can be considered a new line of therapy.

3. Stem cell transplantation (SCT): In patients undergoing >1 SCT, except in the case of a planned tandem SCT with a predefined interval (such as 3 months), each SCT (autologous or allogeneic) can be considered a new line of therapy regardless of whether the conditioning regimen used is the same or different. Generally, planned tandem SCT is considered 1 line. Planned induction and/or consolidation, maintenance with any SCT (frontline, relapse, autologous or allogeneic) is generally considered 1 line of therapy.

In some embodiments, the multiple myeloma is difficult to treat. In some embodiments, the individual has refractory multiple myeloma. In some embodiments, an individual with refractory multiple myeloma is one who was refractory to all prior therapies (or prior lines of therapy), but achieved at least a minimal response (MR) to one prior therapy (or line of therapy). In some embodiments, a minimal response (MR) is defined according to International Myeloma Working Group criteria (see, e.g., Kumar et al. (2016) “International Myeloma Working Group consensus criteria for response and minimal residual disease assessment in multiple myeloma.” Lancet Oncol. 17(8):e328-e346; Durie et al. (2006) “International uniform response criteria for multiple myeloma. Leukemia. 20: 1467-1473; and Table 14 herein). In some embodiments, an individual with refractory multiple myeloma is one who was non-responsive to a prior therapy (or prior line of therapy). In some embodiments, “non-responsive” to a therapy (or line of therapy) for multiple myeloma means that the individual failed to achieve a minimal response (MR) to the therapy (or line of therapy) for multiple myeloma. In some embodiments “non-responsive” to a therapy (or line of therapy) for multiple myeloma means that the individual has demonstrated progressive disease during the therapy (or line of therapy) for multiple myeloma. In some embodiments, an individual with refractory multiple myeloma is one who demonstrated progressive disease within the 60 days from the end of the last therapy for multiple myeloma.

In In some embodiments, the individual has failed prior treatment (such as lenalidomide and/or a proteasome inhibitor) for multiple myeloma. In some embodiments, “failing” a prior treatment means that the individual has demonstrated disease progression (e.g. according to the criteria in Table A) while on the treatment (such as treatment with lenalidomide and/or a proteasome inhibitor) or within 60 days from end of treatment (such as treatment with lenalidomide and/or a proteasome inhibitor). In some embodiments, “failing” a prior treatment for multiple myeloma means that the individual had demonstrated a partial response (PR) or better (e.g., according to the criteria in Table A) to treatment (such as treatment with lenalidomide and/or a proteasome inhibitor), but exhibited disease progression within 6 months after discontinuing the treatment (e.g., as treatment with lenalidomide and/or a proteasome inhibitor). In some embodiments, “failing” a prior treatment for multiple myeloma means that this individual developed toxicity/intolerance after a minimum of 2 consecutive cycles of a treatment regimen (e.g., a treatment regimen containing lenalidomide and/or a proteasome inhibitor (bortezomib, carfilzomib, ixazomib)). In some embodiments, intolerance to a proteasome-containing regimen refers to the individual (e.g., an individual who did not have peripheral neuropathy prior to starting the regimen) developing peripheral neuropathy or neuropathic pain. In some embodiments, intolerance to a lenalidomide-containing regimen refers to the individual developing a severe rash.

In some embodiments, the individual has relapsed and refractory multiple myeloma. In some embodiments, an individual with relapsed and refractory multiple myeloma is one who relapsed from at least one prior therapy (or line of therapy) for multiple myeloma and was refractory to the most recent therapy (or line of therapy) for multiple myeloma. In some embodiments, the individual with relapsed and refractory multiple myeloma is one who relapsed from at least one prior therapy (or line of therapy) for multiple myeloma, was refractory to the most recent therapy (or line of therapy) for multiple myeloma, and was refractory to one or more therapies (or lines of therapy) prior to the most recent therapy (or line of therapy) for multiple myeloma. In some embodiments, an individual with relapsed or refractory multiple myeloma is one who demonstrated progressive disease within 60 days after the end of the most recent therapy (or line of therapy).

In some embodiments, the individual was refractory to the most recent prior therapy (or line of therapy).

In some embodiments, the individual has relapsed/refractory multiple myeloma (RRMM) with measurable disease (e.g., serum M protein≥0.5 g/dL measured using serum protein immunoelectrophoresis and/or urine M protein≥200 mg/24 hours measured using urine protein immunoelectrophoresis and/or serum free light chain (FLC) (i.e., FLC assay≥10 mg/dl (≥100 mg/L) and an abnormal serum FLC ratio (<0.26 or >1.65)) who has received at least 2 prior therapies, including lenalidomide and a proteasome inhibitor (e.g., bortezomib, carfilzomib, or ixazomib) and was refractory to the last line of therapy (i.e., most recent line of therapy). In some embodiments, the individual has adequate renal, hepatic and bone marrow function.

In some embodiments, the individual has a poor prognosis. In some embodiments of the methods and uses provided herein, the individual has received at least one, at least two, at least three, at least four prior therapies (or lines of therapy), or more than four prior therapies (or lines of therapy), e.g., at least any one of 5, 6, 7, 8, 9, 10, or 11 prior therapies (or lines of therapy) for multiple myeloma.

In some embodiments, the individual has undergone at least one prior therapy (or line of therapy) with lenalidomide. In some embodiments, the prior lenalidomide therapy (or line of therapy) comprised at least two consecutive cycles of lenalidomide. In some embodiments, the individual failed (e.g., was non-responsive to) a prior lenalidomide therapy (or a line of therapy). In some embodiments, an individual who failed a prior lenalidomide therapy (or a line of therapy) did not achieve at least a minimal response (MR) during the therapy (or line of therapy) with lenalidomide. In some embodiments, an individual who failed a prior lenalidomide therapy (or a line of therapy) demonstrated progressive disease (PD) during the therapy (or line of therapy) with lenalidomide. As noted elsewhere herein, in some embodiments, “minimal response” and “progressive disease” are assessed according to the criteria in Kumar et al. (2016) “International Myeloma Working Group consensus criteria for response and minimal residual disease assessment in multiple myeloma.” Lancet Oncol. 17(8): e328-e346 and Durie et al. (2006) “International uniform response criteria for multiple myeloma. Leukemia. 20: 1467-1473 (see also Table 14 herein). In some embodiments, prior lenalidomide therapy was administered during the first, second, third, fourth, fifth, sixth, and/or later therapy (or line of therapy) for multiple myeloma (i.e., prior to a treatment described herein comprising administration of the anti-CD38 antibody (e.g., isatuximab)). In some embodiments, the individual was refractory to lenalidomide. In some embodiments, the prior lenalidomide was administered to the individual as a single agent. In some embodiments, the prior lenalidomide was administered to the individual in conjunction with at least one additional agent.

In some embodiments, the individual has undergone at least one prior therapy (or at least one prior line of therapy) with a proteasome inhibitor. In some embodiments, the proteasome inhibitor is selected from the group consisting of: bortezomib, carfilzomib, and ixazomib. In some embodiments, the prior therapy (or line of therapy) with the proteasome inhibitor comprised at least two consecutive cycles of the proteasome inhibitor. In some embodiments, the individual failed (e.g., was non-responsive to) a prior proteasome inhibitor therapy (or a prior line of therapy). In some embodiments, an individual who failed a prior therapy (or a line of therapy) with the proteasome inhibitor did not achieve at least a minimal response (MR) during the therapy (or line of therapy) with the proteasome inhibitor. In some embodiments, an individual who failed a prior therapy (or a line of therapy) with a proteasome inhibitor demonstrated progressive disease (PD) during the therapy (or line of therapy) with the proteasome inhibitor. In some embodiments, the prior proteasome inhibitor therapy was administered during the first, second, third, fourth, fifth, sixth, and/or later therapy (or line of therapy) for multiple myeloma (i.e., prior to a treatment described herein comprising administration of the anti-CD38 antibody (e.g., isatuximab)). In some embodiments, the individual was refractory to the proteasome inhibitor (e.g., such as one or more proteasome inhibitors). In some embodiments, the prior proteasome inhibitor therapy was administered to the individual as a single agent. In some embodiments, the prior proteasome inhibitor therapy was administered to the individual in conjunction with at least one additional agent.

In some embodiments, the individual has received at least two prior therapies (or lines of therapy) including lenalidomide (as described elsewhere herein) and a proteasome inhibitor (as described elsewhere herein). In some embodiments, the individual also demonstrated disease progression while on the most recent prior therapy or after completion of the most recent prior therapy (e.g., prior to a treatment described herein comprising administration of the anti-CD38 antibody (e.g., isatuximab). In some embodiments, the lenalidomide and the proteasome inhibitor were administered to the individual in combination. In some embodiments, the individual previously achieved a partial response (PR) or greater to lenalidomide and/or the proteasome inhibitor (given alone or in combination), but demonstrated progressive disease (PD) within 6 months of the end of the therapy (or line of therapy) with lenalidomide and/or the proteasome inhibitor.

In some embodiments, the individual has received prior therapy (or at least one prior line of therapy) with pomalidomide.

In some embodiments, the individual has a respiratory, thoracic, and/or mediastinal disorder. In some embodiments, the individual has chronic obstructive pulmonary disorder (COPD). In some embodiments, the individual is diagnosed with COPD prior to the start of a treatment described herein comprising administration of the anti-CD38 antibody (e.g., isatuximab). In some embodiments, the individual develops and/or is diagnosed with COPD after the start of a treatment described herein comprising administration of the anti-CD38 antibody (e.g., isatuximab). In some embodiments, the individual has asthma. In some embodiments, the individual is diagnosed with asthma prior to the start of a treatment described herein comprising administration of the anti-CD38 antibody (e.g., isatuximab). In some embodiments, the individual develops and/or is diagnosed with asthma after the start of a treatment described herein comprising administration of the anti-CD38 antibody (e.g., isatuximab). In some embodiments, the individual has (e.g., experiences) bronchospasms. In some embodiments, the individual experienced bronchospasms prior to the start of a treatment described herein comprising administration of the anti-CD38 antibody (e.g., isatuximab). In some embodiments, the individual develops bronchospasms after the start of a treatment described herein comprising administration of the anti-CD38 antibody (e.g., isatuximab). In some embodiments, the individual has one or more of the following: bronchial hypersensitivity, cough, dyspnea, dyspnea at rest, dyspnea exertional, emphysema, hypoxia, lung infiltration, oropharyngeal pain, pleural effusion, pleuritic pain, pulmonary embolism, pulmonary hypertension, allergic rhinitis, and rhinorrhea. In some embodiments, the individual experienced one or more of bronchial hypersensitivity, cough, dyspnea, dyspnea at rest, dyspnea exertional, emphysema, hypoxia, lung infiltration, oropharyngeal pain, pleural effusion, pleuritic pain, pulmonary embolism, pulmonary hypertension, allergic rhinitis, and rhinorrhea prior to the start of a treatment described herein comprising administration of the anti-CD38 antibody (e.g., isatuximab). In some embodiments, the individual develops one or more of bronchial hypersensitivity, cough, dyspnea, dyspnea at rest, dyspnea exertional, emphysema, hypoxia, lung infiltration, oropharyngeal pain, pleural effusion, pleuritic pain, pulmonary embolism, pulmonary hypertension, allergic rhinitis, and rhinorrhea after the start of a treatment described herein comprising administration of the anti-CD38 antibody (e.g., isatuximab).

In some embodiments, the individual does not have primary refractory multiple myeloma. In some embodiments, an individual with primary refractory multiple myeloma is one who has never achieved at least a minimal response (MR) with any therapy (or line of therapy) during the disease course. In some embodiments, the individual does not have free light chain (FLC) measurable disease only. In some embodiments, the individual has not received prior treatment with an anti-CD38 antibody. In some embodiments, the individual has not received a prior therapy (or a prior line of therapy) with isatuximab. In some embodiments, the individual has not demonstrated progressive disease (PD) during a prior therapy (or prior line of therapy) with an anti-CD38 antibody. In some embodiments, the individual has not demonstrated PD within 60 days after the end of a therapy (or line of therapy) with an anti-CD38 antibody. In some embodiments, the individual has not received a prior therapy (or a prior line of therapy) with pomalidomide. In some embodiments, the individual has not received prior allogenic hematopoietic stem cell transplantation.

In some embodiments, the individual is less than 65 years of age. In some embodiments, the individual is between 65 and less than 75 years of age. In some embodiments, the individual is 75 years of age or older. In some embodiments, the individual is female (e.g. a fertile female of childbearing age). In some embodiments, the individual has Eastern Cooperative Oncology Group (ECOG) Performance Status score of no more than 0, no more than 1, or no more than 2. In some embodiments, the individual is Stage I, Stage II, or Stage III according to the Multiple Myeloma International Stating System (ISS).

### Single Agent and Combination Treatments

In some embodiments, a method of treatment described herein comprises the administration of an anti-CD38 antibody (e.g., isatuximab) as a single agent (e.g., as monotherapy). In some embodiments, the anti-CD38 antibody is administered in conjunction with at least one additional agent (such as two or more additional agents). The additional agent can be a small molecule drug or a biologic, such as an antibody.

In some embodiments, the at least one additional agent comprises an immunomodulatory drug (IMiD®). In some embodiments, the IMiD® administered in conjunction with the anti-CD38 antibody (e.g., isatuximab) is thalidomide, lenalidomide, and/or pomalidomide. In some embodiments, the anti-CD38 antibody (e.g., isatuximab) and the immunomodulatory drug are administered further in conjunction with a corticosteroid, e.g., dexamethasone or prednisone. In some embodiments, the anti-CD38 antibody (e.g., isatuximab) is administered in conjunction with lenalidomide and dexamethasone. In some embodiments, the anti-CD38 antibody (e.g., isatuximab) is administered in conjunction with pomalidomide and dexamethasone. In some embodiments, the anti-CD38 antibody (e.g., isatuximab), the immunomodulatory drug (e.g. lenalidomide), and the corticosteroid (e.g., dexamethasone) are administered further in conjunction with an anti-coagulation agent (e.g., aspirin, warfarin, or heparin).

In some embodiments, the at least one additional agent comprises a proteasome inhibitor. In some embodiments, the proteasome inhibitor administered in conjunction with the anti-CD38 antibody (e.g., isatuximab) is bortezomib, carfilzomib, ixazomib citrate, marizomib, and/or oprozomib. In some embodiments, the anti-CD38 antibody (e.g., isatuximab) and the proteasome inhibitor are administered further in conjunction with a corticosteroid, e.g., dexamethasone or prednisone. In some embodiments, the anti-CD38 antibody (e.g., isatuximab) and the proteasome inhibitor, are administered in conjunction with an IMiD® (e.g., thalidomide, lenalidomide, and/or pomalidomide). In some embodiments, the anti-CD38 antibody is administered in conjunction with carfilzomib, lenalidomide, and dexamethasone. In some embodiments, the anti-CD38 antibody is administered in conjunction with bortezomib, lenalidomide, and dexamethasone. In some embodiments, the anti-CD38 antibody (e.g., isatuximab) and the proteasome inhibitor are administered further in conjunction with an alkylating agent (e.g., including, without limitation, cyclophosphamide, cyclophosphamide monohydrate, bendamustine, bendamustine hydrochloride, busulfan, carmustine, lomustine, melphalan, melphalan flufenamide, melphalan hydrochloride, thiotepa, treosulfan). In some embodiments, the anti-CD38 antibody (e.g., isatuximab) is administered in conjunction with bortezomib, lenalidomide, and dexamethasone.

Additionally or alternatively, in some embodiments, the at least one additional agent comprises a histone deacetylase inhibitor (HDAC inhibitor), e.g., without limitation, panobinostat or panobinostat lactate). Additionally or alternatively, in some embodiments, the at least one additional agent comprises an anthracycline, e.g., without limitation, daunorubicin, doxorubicin, doxorubicin hydrochloride, idarubicin, liposomal doxorubicin hydrochloride, mitoxantrone, pegylated liposomal doxorubicin, or pegylated liposomal doxorubicin hydrochloride. Additionally or alternatively, in some embodiments, the at least one additional agent comprises a corticosteroid, e.g., without limitation, betamethasone, betamethasone sodium phosphate, dexamethasone, dexamethasone acetate, dexamethasone sodium phosphate, methylprednisolone, prednisolone, or prednisone. Additionally or alternatively, in some embodiments, the at least one additional agent comprises a vinca alkaloid, e.g., without limitation, vincristine or vincristine sulfate.

### Articles of Manufacture or Kits

In another embodiment of the invention, an article of manufacture or a kit is provided comprising an anti-CD38 antibody (such as isatuximab). In some embodiments, the article of manufacture or kit further comprises package insert comprising instructions for using the anti-CD38 antibody (e.g., isatuximab) to treat or delay progression of multiple myeloma (e.g., refractory multiple myeloma or relapsed and refractory multiple myeloma) in an individual who has received at least two prior therapies for multiple myeloma (e.g., including lenalidomide and a proteasome inhibitor). In some embodiments, the article of manufacture or kit further comprises a package insert or label comprising instructions for administering one or more 10 mg/kg doses of anti-CD38 antibody (such as isatuximab), wherein each dose is in a volume of 250 ml, according to a method described herein. In some embodiments, the article of manufacture or kit further comprises a package insert or label comprising instructions for administering 20 mg/kg anti-CD38 antibody (such as isatuximab).

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLE 1A

### Preliminary Results from a Phase 1 b Study to Evaluate the Feasibility and Safety of Isatuximab Short Duration Fixed Volume Infusion in Combination with Pomalidomide and Dexamethasone for Relapsed and/or Refractory Multiple Myeloma

This example describes a multicenter, open label, non-comparative, Phase 1b study that assessed a simplified infusion administration of isatuximab (I) in combination with pomalidomide and dexamethasone (Pd) using a fixed infusion volume in patients with relapsed/refractory multiple myeloma (RRMM) that have been previously exposed to proteasome inhibitors and immunomodulatory drugs and are relapsed/refractory to the most recent therapy.

### I. Study Objectives

The primary objective of this study was to evaluate the feasibility of isatuximab (I) administered from a fixed infusion volume in combination with pomalidomide and dexamethasone (Pd) as assessed by occurrence of Grade≥3 infusion reactions (IR).

The secondary objectives of this study were: (1) to evaluate the infusion duration for administration of isatuximab in combination with Pd in a fixed infusion volume; (2) to evaluate the safety profile of the Pd combination with isatuximab administration with fixed volume; (3) to evaluate immunogenicity of isatuximab in combination with Pd; and (4) to describe the efficacy of the combination of isatuximab with Pd in terms of overall response rate (ORR, i.e., CR+VGPR+PR) and clinical benefit rate (CBR, i.e., CR+VGPR+PR+MR) based on the International Myeloma Working Group (IMWG) response criteria and the duration of response in RRMM patients (see Table 14) (Kumar et al., (2016) Lancet Oncol. 17(8):e328-e346 and Durie et al. (2006) “International uniform response criteria for multiple myeloma. Leukemia. 20: 1467-1473.).

The Exploratory Objectives of this study were: (1) to investigate the multiple myeloma molecular subtype (as defined by cytogenetics) and clinical response; (2) to investigate the relationship between immune genetic determinants, immunophenotype and parameters of clinical response; (3) to assess minimal residue disease (MRD) patients achieving a complete response (CR) and correlate with clinical outcome (see Table 14); and (4) to investigate the potential isatuximab interference with the M protein assessment in immunoelectrophoresis and immunofixation assays.

### II. Study Population

A. Inclusion Criteria

Eligible patients were considered for inclusion in this study if they met all of the following criteria:

Patient had been previously diagnosed with multiple myeloma (MM) based on standard criteria and required treatment because MM has relapsed following a response, according to IMWG criteria.

Patient received at least two previous therapies, including lenalidomide and proteasome inhibitor, and had demonstrated disease progression on last therapy or after completion of the last therapy.

Patient had measurable disease defined as at least one of the following:
Serum M protein≥0.5 g/dL (≥5 g/L).
Urine M protein≥200 mg/24 hours.
Serum free light chain (sFLC) assay: Involved FLC assay≥10 mg/dL (≥100 mg/L) and an abnormal serum FLC ratio (<0.26 or >1.65).

All patients enrolled into this trial were registered in and complied with all requirements of the POMALYST REMS™ program (www(dot)pomalystrems(dot)com).

B. Exclusion Criteria

Patients that met any of the following criteria were ineligible for this study:

Male or female patients less than 18 years of age.

Patients diagnosed or treated for another malignancy within 3 years prior to enrollment, with the exception of complete resection of basal cell carcinoma or squamous cell carcinoma of the skin, an in-situ malignancy, or low risk prostate cancer after curative therapy.

Patients with an Eastern Cooperative Oncology Group (ECOG) performance status score greater than 2, or life expectancy less than or equal to 3 months.

Clinical Laboratories Exclusion Criteria: Patients were excluded if the screening laboratory results were as follows:
Absolute neutrophil count (ANC)<1000 cells/μl (1.0×10⁹/L). Growth factor could not be used within the previous 7 days.
Aspartate aminotransferase (AST/SGOT) or Alanine aminotransferase (ALT/SGPT)≥2.5×upper limit of normal (ULN).
Platelet count<50 000 cells/μl (50×10⁹/L) without platelet transfusion in the previous 7 days.
Total bilirubin≥1.5×ULN.
Calculated creatinine clearance (CrCl) using <30 mL/min according to the MDRD equation:

Glomerular filtration rate (mL/min/1.73 m²)=175×(Scr)-1.154×(Age)−0.203×(0.742 if Female)×(1.212 if African-American), where Scr is serum creatine in mg/dL and Age is in years of age.
Serum calcium (corrected for albumin) level above the ULN range. Treatment of hypercalcemia was allowed and patients were allowed to enroll in this study if hypercalcemia returned to normal with standard treatment.

Primary refractory or intolerant to prior therapy with any anti-CD38 monoclonal antibody (MoAb) or had disease progression (after achieving a response of ≥MR) during anti-CD38 MoAb, administered as last therapy.

Received any investigational drug within 14 days or 5 half-lives of the investigational drug, whichever is longer.

Prior anti-cancer therapy within 14 days.

Any Grade>1 adverse reaction unresolved from previous treatments according to the NCI CTC AE v. 4.03. The presence of alopecia or peripheral neuropathy Grade≤2 without pain was allowed.

Previous allogeneic stem cell transplantation with active Graft Versus Host Disease (GVHD) or being under immunosuppressive therapy in the last 2 months prior to inclusion in the trial.

Daily requirement for corticosteroids (equivalent to 10 mg/day prednisone for more than 7 consecutive days except for inhalation corticosteroids and patients being treated for adrenal insufficiency/replacement therapy).

Patient was known to be human immunodeficiency virus (HIV) positive, Hepatitis B surface antigen-positive, or had an active hepatitis C infection.

Any clinically significant, uncontrolled medical condition that, in the Investigator's opinion, would have imposed excessive risk to the patient or may have interfered with compliance or interpretation of the study results.

History of erythema multiforme or severe hypersensitivity to prior IMiDs®.

Hypersensitivity or history of intolerance to IMiDs®, dexamethasone, sucrose, histidine (as base and hydrochloride salt) and polysorbate 80 or any of the components of study therapy that are not amenable to premedication with steroids and H2 blockers or would prohibit further treatment with these agents.

Hypersensitivity to boron and or mannitol (i.e., where the investigational medicinal products (IMPs) and/or non-investigational medicinal products (NIMPs) contain boron and/or mannitol)

Inability to tolerate thromboprophylaxis.

### III. Study Design

A. Primary Endpoint

The primary endpoint of this study was the incidence of IRs of Grade≥3 reported during the first six infusions of isatuximab, from a fixed infusion volume in combination with Pd.

B. Secondary Endpoints

The secondary endpoints of this study were:

i. Duration of Infusion Time

Infusion duration was measured from the start of isatuximab infusion to the end of isatuximab infusion, regardless of temporary stop/interruption.
ii. Safety and Immunogenicity

Safety was assessed through collection of treatment-emergent adverse events (TEAE) and changes in laboratory parameters (hematology, biochemistry, and urinalysis), vital signs (heart rate, blood pressure, and body weight), ECG, physical exam, and ECOG PS. Patients were assessed for the presence of human anti-drug antibodies (ADA) to isatuximab. An adverse event was defined as any untoward medical occurrence in a patient administered a pharmaceutical product, and which does not necessarily have a causal relationship with the study treatment.
iii. Efficacy

Efficacy was assessed according to the updated IMWG Response Criteria (see Kumar S. et al., Lancet Oncol. 2016; 17(8): e328-e46; Durie et al. (2006) “International uniform response criteria for multiple myeloma. Leukemia. 20: 1467-1473; and Table 14 herein) to evaluate the percentage of patients with objective response (overall response rate), with Clinical Benefit response (CBR) using IMWG defined response criteria, and duration of response.

C. Exploratory Endpoints

Bone marrow and/or blood samples were analyzed for genomic profiling and multiple myeloma molecular subtype (using cytogenetics) and bone marrow for the levels of CD38 mRNA. These markers were correlated with clinical response. In addition, cytogenetic analysis was carried out on blood samples for immune genetic determinants (such as Fc polymorphisms, Human Leukocyte Antigen (HLA) and Killer Immunoglobulin-like Receptors (KIR), etc.) correlated with clinical response. The correlation of immunophenotype (such as B-cell, T-cell, and Natural Killer (NK)-cell subsets) in peripheral blood with parameters of clinical response was also assessed. Finally, MRD by sequencing was assessed in CR patients and correlated with clinical outcome.

D. Statistical Methods

The incidence of grade≤3 IRs reported determined the sample size. With a total of approximately 40 patients, the fixed infusion volume of isatuximab will not have been considered feasible if the lower bound of the 95% CI is >5.5%; i.e., if ≥6 patients have grade ≥3 IRs.

The statistical evaluation for all analyses was descriptive and performed based on all treated patients who completed at least 6 isatuximab infusions or terminated study treatment early (with definitive end of treatment). Continuous data was summarized using number of available data, mean, standard deviation, median, minimum, and maximum. Categorical and ordinal data was summarized using number and percentage of patients. Number (percentage) of patients with Grade≥3 IR within the first six isatuximab infusions among patients evaluable for IR assessment was analyzed with 95% confidence interval using Clopper-Pearson method.

E. Duration of Study Participation

The study duration for an individual patient included a screening period for inclusion of up to 21 days. The treatment period continued until disease progression, unacceptable AE or other reason for discontinuation. Patients were followed for a minimum of 30 days after the last use of investigational medicinal product/non-investigational medicinal product (IMP/NIMP) or more than 30 days in case of unresolved IMP/NIMP related adverse events (AE). For all patients, any study treatment-related adverse events, and all serious adverse events (SAE) (regardless of their causal relationship to study treatment) ongoing at the time of study treatment discontinuation were followed during the follow-up period until resolution or stabilization. The primary analysis cut-off date occurred when the last enrolled patient completed six infusions. The final analysis cut-off date was 10 months after the date of the first dose of the last enrolled patient.

No samples were collected for ADA analyses after 10 cycles. If the last ADA sample was positive or inconclusive, additional ADA sampling was done 3 months later. No further ADA was sampled, even if the 3-month sample was positive.

F. Fixed Volume Infusion Schedule

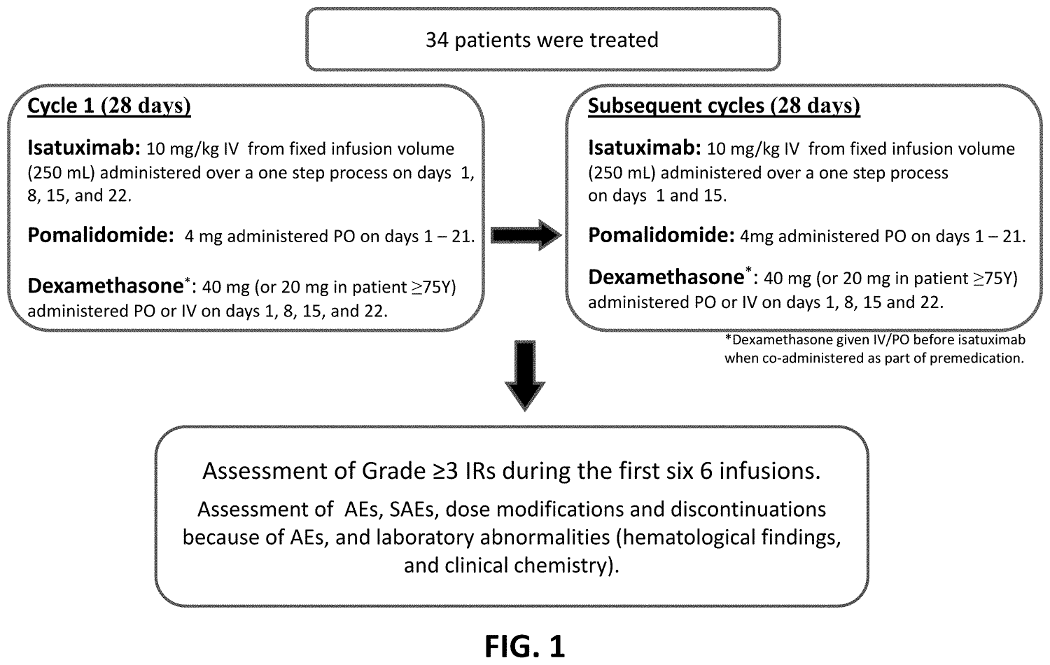

As shown in FIG. 1, Isatuximab was administered intravenously (IV) over a one-step process at the selected dose of 10 mg/kg from a fixed volume of 250 mL with an infusion rate expressed in ml/h. The fixed volume was administered on days 1, 8, 15, and 22 of the first 28-day cycle. During each subsequent 28-day cycle, the fixed volume was administered on days 1 and 15. The patient's weight was measured prior to each cycle to allow calculation of the isatuximab dose. Pomalidomide was administered orally on days 1-21 of every 28-day cycle. Dexamethasone was administered orally or intravenously on Days 1, 8, 15, and 22 of every 28-day cycle. When dexamethasone was co-administered as part of premedication, it was administered orally or intravenously before administration of isatuximab. As described in further detail below, all patients received pre-treatment prophylaxis for hypersensitivity reactions.

Grade 3 or greater IRs were assessed during the first six isatuximab infusions. The study treatment of patients that experienced a Grade ≥3 IR was permanently discontinued and appropriate supportive therapy was administered. After the sixth infusion, patients continued on study treatment until disease progression, unacceptable toxicity, or other reasons for discontinuation. As described in further detail below, patients initiated additional cycles if they meet the criteria for the initiation of a new cycle of therapy.

G. Fixed Volume Infusion Rates

First infusion: The first infusion was initiated at an infusion rate of 25 mL/hour. In the absence of IRs after 1 hour of infusion, the infusion rate was increased by 25 mL/hour increments every 30 minutes, to a maximum infusion rate of 150 mL/hour. In case of Grade 2 IRs during the first infusion, the infusion was restarted at one-half the rate (12.5 mL/hour) of the initial infusion rate upon improvement of IRs to Grade≤1. If symptoms did not recur after 30 minutes, the infusion rate was increased in 25 mL/hour increments every 30 minutes, until the total volume was infused.

Second infusion: The second infusion was initiated at a rate of 50 mL/hour. In the absence of Grade 2 IRs after 30 minutes of infusion, the rate was increased by 100 mL/hour for 30 minutes, then 200 mL/hour for 30 minutes, and then 300 mL/hour until the total volume was infused. In case of Grade 2 IRs during the second infusion, infusions were restarted at one-half the rate of the initial infusion rate (25 mL/hour) when the IRs improved to Grade≤1. If symptoms did not recur after 30 minutes, the infusion rate was increased in 50 mL/hour increments every 30 minutes, until the total volume is infused.

Third and subsequent infusions: The third and subsequent infusions were initiated at a fixed infusion rate of 200 mL/hour, until the total volume was infused. In case of Grade 2 IRs during the third infusion, the infusion was restarted at one-half of the infusion rate (100 mL/hour) when the IRs improved to Grade≤1. If symptoms did not recur after 30 minutes, the infusion rate was increased in 50 mL/hour increments every 30 minutes, until the total volume was infused.

H. Investigational Medicinal Products (IMPs)

i. Isatuximab

Isatuximab is an anti-CD38 antibody comprising a heavy chain that comprises the sequence of SEQ ID NO: 10 and a light chain comprising the sequence of SEQ ID NO: 11. Isatuximab was provided as a sterile, non-pyrogenic, injectable, colorless concentrate in 30 mL glass vials fitted with elastomeric closure. Each vial contained 20 mg/mL (500 mg/25 mL) isatuximab in 20 mM histidine, 10% (w/v) sucrose, 0.02% (w/v) polysorbate 80, pH 6.0 buffer. Vials with white to white-off particulates were permitted. Each vial contained a nominal content of 500 mg of isatuximab.

Isatuximab was administered at the selected dose of 10 mg/kg IV (from a fixed volume of 250 mL) on days 1, 8, 15, and 22 of the first 28-day cycle. During subsequent cycles, isatuximab was administered on days 1 and 15. The patient's weight was measured prior to each cycle to allow calculation of the isatuximab dose.

For administration to patients, the appropriate volume of isatuximab was diluted in an infusion bag of 0.9% sodium chloride solution. No protection from light is required for storage in the infusion bags. The Investigational medicinal product was stored at +2° C. to +8° C.

ii Pomalidomide

Pomalidomide capsules were administered orally from days 1-21 of each 28 day-cycle at the dose of 4 mg, according to the pomalidomide prescribing information (available at the web site: www(dot)accessdata.fda(dot)gov/drugsatfda_docs/label/2013/2040261b1(dot)pdf).

iii. Dexamethasone

Dexamethasone (40 mg for patients younger than 75 years of age; 20 mg for patients of 75 years of age or older) was administered either orally (PO) or by IV infusion on days 1, 8, 15, and 22 of each 28-day cycle.

### I. Non-Investigational Medicinal Products (NIMPs)—Premedication for the Prevention of Infusion Reactions (IRs)

Patients routinely received pre-medications prior to isatuximab infusion to reduce the risk and severity of IRs commonly observed with monoclonal antibodies. The recommended premedication agents were: diphenhydramine, 25-50 mg, administered IV (or equivalent, intravenous route was preferred for at least the first 4 infusions); ranitidine, 50 mg, administered IV (or equivalent); and acetaminophen, 650-1000 mg, administered PO 15-30 minutes (but no longer than 60 minutes) prior to isatuximab infusion. Once the premedication regimen was completed, the isatuximab infusion was started.

In addition, 40 mg of dexamethasone (or 20 mg in case of patient≥75 years of age) were administered as part of premedication PO or IV before administration of isatuximab. Because dexamethasone was also an IMP administered on days 1, 8, 15, and 22 of each 28-day cycle, during the days of isatuximab infusions, dexamethasone was administered only once before isatuximab infusion, and the single administration was used for both premedication and study treatment. The order of administration of premedications is provided below:

When dexamethasone was administered PO, the following order was used:
Dexamethasone 40 mg PO (or 20 mg PO for patients >75 years of age).
Acetaminophen (paracetamol) 650 mg to 1000 mg PO.
Ranitidine 50 mg IV (or equivalent).
Diphenhydramine 25 mg to 50 mg IV (or equivalent).

When dexamethasone was administered IV, the following order was used:
Acetaminophen (paracetamol) 650 mg to 1000 mg PO.
Ranitidine 50 mg IV (or equivalent).
Diphenhydramine 25 mg to 50 mg IV (or equivalent).
Dexamethasone 40 mg IV (or 20 mg IV for patients≥75 years of age).

For patients who could not tolerate dexamethasone during study treatment or dexamethasone being prematurely stopped, methylprednisolone 100 mg IV was administered as premedication only. However, both drugs were not used at the same time for premedication purposes.

J. Dose Delays, Omissions, and/or Modifications

i. Isatuximab

No dose reductions were authorized for isatuximab. If dose reduction of isatuximab occurred, the patient was withdrawn from study treatment unless a clear benefit from therapy was observed.

Patients had a dose of isatuximab omitted if toxicity occurred within a cycle and did not recover the day of planned infusion. In such cases, infusion could be delayed up to 3 days. Otherwise, the infusion was omitted and patients received the next isatuximab infusion after recovery of the toxicity. No more than two consecutive isatuximab infusions omissions per patient were permitted.

Stopping and altering the infusion rate of isatuximab was permitted in response to IRs as follows:

Grade 1 IRs: Infusion interruption or intervention was not indicated for patients experiencing a Grade 1 IR. However, if the infusion was stopped as deemed necessary, the IR was classified as Grade 2.

Grade 2 IRs: Infusion interruption and additional premedications as needed were indicated for patients experiencing Grade 2 IRs. Once a Grade 2 IR improved to Grade≤1, the infusion was restarted at one half the original infusion rate under close monitoring and supportive care as needed. If symptoms did not recur after 30 minutes, the infusion rate was increased as follows:
25 mL/hour increments every 30 minutes, until the total volume was infused during the first infusion.
50 mL/hour increments every 30 minutes, until the total volume was infused during the second infusion.
50 mL/hour increments every 30 minutes, until the total volume was infused during the third and subsequent infusions.

Grade 3 or 4 IRs: Patients with Grade 3 or 4 IRs had isatuximab treatment permanently discontinued and appropriate therapy was administered.

ii Pomalidomide

One or several doses of pomalidomide were omitted within a cycle if toxicity occurred and did not recover the day of the planned infusion/administration. The dose of pomalidomide was adjusted from the starting dose of 4 mg to 3 mg for the first dose reduction, 2 mg for the second dose reduction, and 1 mg for the third dose reduction. No more than 3 dose reductions of pomalidomide per patient were permitted. Once reduced, dosing was never re-escalated. If strong inhibitors of CYP1A2 were co-administered in the presence of strong inhibitors of CYP3A4 and inhibitors of P-glycoprotein, pomalidomide dose was reduced by 50%. If pomalidomide was prematurely permanently discontinued, then isatuximab was continued until disease progression or unacceptable toxicity or patient's refusal of further treatment.

iii. Dexamethasone

One or several doses of dexamethasone were omitted within a cycle if toxicity occurred and did not recover the day of the planned infusion/administration. For patients younger than 75 years of age, the starting dose of 40 mg of dexamethasone was adjusted to 20 mg for the first dose reduction, 12 mg for the second dose reduction, 8 mg for the third dose reduction, and dexamethasone was discontinued if further reductions were needed. For patients 75 years of age or older, the starting dose of 20 mg of dexamethasone was adjusted to 12 mg for the first dose reduction, 8 mg for the second dose reduction, 4 mg for the third dose reduction, and dexamethasone was discontinued if further reductions were needed. Once reduced, dosing was never re-escalated. If dexamethasone was prematurely permanently discontinued, then isatuximab was continued until disease progression or unacceptable toxicity or patient's refusal of further treatment.

K. Concomitant Therapy

Standard prophylactic medication with antihistaminic and antipyretic agents without post-infusion corticosteroid prophylaxis was given. Premedication after 4 infusions was reconsidered. Anti-coagulation prophylaxis was required after an assessment of each patient's underlying risk factors. Unless there was an excess risk of bleeding, all patients received standard (e.g., prophylactic) anti-thrombotic treatment unless contraindicated.

L. Initiation of a New Cycle

A cycle of study treatment was started if the following criteria were met:
ANC≥1,000/mm³. G-CSF use was permitted during all cycles and was allowed on the same day as treatment administration.
Platelet count≥50,000/mm³. Platelet transfusions were permitted during all cycles and allowed on the same day as treatment administration.
Any IMP-related AE had reduced to less than Grade 1 severity or baseline.

If the above criteria were not met on day 1 of the scheduled cycle, patients were re-evaluated weekly. Patients who did not meet the above criteria within 14 days of day 1 of the scheduled cycle were discontinued from study treatment.

### IV. Results

A. Patient Characteristics

All patients who completed at least 6 isatuximab infusions (2 cycles) or terminated study treatment early (with definitive end of treatment) were included in the results. Thus, a total of 34 patients were included, of which 24 (70.6%) were still on treatment at the end of the study and 10 (29.4%) had terminated treatment early (Table 1).

As shown in Table 1, the reasons for definitive study treatment discontinuation among the 10 (29.4%) patients who had terminated treatment early were: disease progression (7 patients) and AEs (3 patients). One patient prematurely discontinued pomalidomide treatment, and no patient prematurely discontinued dexamethasone treatment.

TABLE 1
Number of patients in all treated patients and reasons for treatment discontinuation.

| | Isatuximab | |
|---|---|---|
| | (dose level and schedule) + | |
| | pomalidomide/dexamethasone | |
| | 10 mg/kg QW/Q2W (N = 34) | |
| Off treatment | 10 | (29.4%) |
| Reasons for definitive treatment discontinuation | | |
| Adverse event | 3 | (8.8%) |
| Disease progression | 7 | (20.6%) |
| Reasons for premature discontinuation of pomalidomide | | |
| Adverse event | 1 | (2.9%) |
| Ongoing treatment | 24 | (70.6%) |

Table 2 provides a summary of the demographic characteristics of the 34 treated patients. The median age was 64 years (range 46 to 85 years), with the majority of the patients being aged <65 years (55.9%). There were 18 female and 16 male patients. The majority of patients were White (88.2%) and not Hispanic or Latino (85.3%). All patients had ECOG PS of 0 or 1, except one patient (2.9%) who had an ECOG PS 2. At study entry, patients had body weights ranging from 40 kg to 121 kg with a median of 89.1 kg.

TABLE 2
Demographic characteristics of all treated patients.

| | Isatuximab | |
|---|---|---|
| | (dose level and schedule) + | |
| | pomalidomide/dexamethasone | |
| | 10 mg/kg QW/Q2W (N = 34) | |
| Age(years) | | |
| Number | 34 | |
| Mean (SD) | 63.1 | (10.6) |
| Median | 64.0 | |
| Min:Max | 46:85 | |
| Age by category | | |
| <65 years | 19 | (55.9%) |
| ≥65 - <75 years | 10 | (29.4%) |
| ≥75 years | 5 | (14.7%) |
| Race [n(%)] | | |
| Number | 34 | |
| White | 30 | (88.2%) |
| Black or African American | 2 | (5.9%) |
| Asian | 1 | (2.9%) |
| Other | 1 | (2.9%) |
| Ethnicity [n(%)] | | |
| Number | 33 | |
| Hispanic or Latino | 4 | (11.8%) |
| Not Hispanic or Latino | 29 | (85.3%) |
| Missing | 1 | (2.9%) |
| Gender [n(%)] | | |
| Number | 34 | |
| Male | 16 | (47.1%) |
| Female | 18 | (52.9%) |
| Weight (kg) | | |
| Number | 34 | |
| Mean (SD) | 83.1 | (23.3) |
| Median | 89.1 | |
| Mm:Max | 40:121 | |
| ECOG PS [n(%)] | | |
| Number | 34 | |
| 0 | 10 | (29.4%) |
| 1 | 23 | (67.6%) |
| 2 | 1 | (2.9%) |

Note: Reported numbers correspond to the count of patients with non-missing data used for calculation of the percentage.

As shown in Table 3, at study entry, 15 (44.1%), 10 (29.4%), and 5 (14.7%) patients had International Staging System (ISS) criteria of Stage I, II, and III, respectively. The ISS stage of 4 patients (11.8%) was unknown. Most patients (67.6%) had measurable serum M-protein. Patients had a median of 12.6% (range 0% to 96.0%) plasma cells in bone marrow, with 47.1% of patients having 20% to 50% bone marrow plasma cells. Most patients (67.6%) had bone lesions at baseline, and 10 (29.4%) patients had plasmacytoma present at baseline.

TABLE 3
Disease characteristics at study entry of all treated patients.

| | Isatuximab | |
|---|---|---|
| | (dose level and schedule) + | |
| | pomalidomide/dexamethasone | |
| | 10 mg/kg QW/Q2W (N = 34) | |
| ISS at study entry [n(%)] | | |
| Stage I | 15 | (44.1%) |
| Stage II | 10 | (29.4%) |
| Stage III | 5 | (14.7%) |
| Missing | 4 | (11.8%) |
| Measurable paraprotein at baseline [n(%)] | | |
| Serum M-Protein | 23 | (67.6%) |
| Urine M-Protein | 1 | (2.9%) |
| Kappa Light Chain | 6 | (17.6%) |
| Lambda Light Chain | 2 | (5.9%) |
| Missing | 2 | (5.9%) |
| Bone marrow plasma cells (%) | | |
| Number | 34 | |
| Mean (SD) | 30.11 | (32.00) |
| Median | 12.60 | |
| Min:Max | 0.0:96.0 | |
| Bone marrow plasma cells (%) by category | | |
| 0 | 1 | (2.9%) |
| (0-5) | 8 | (23.5%) |
| [5-20) | 9 | (26.5%) |
| [20-50) | 7 | (20.6%) |
| ≥50 | 9 | (26.5%) |
| Missing | 0 | |
| Plasmacytoma at baseline [n(%)] | 10 | (29.4%) |
| Bone lesions at baseline [n(%)] | 23 | (67.6%) |

The most frequent conditions reported in medical histories were: hypertension (18 patients, 52.9%), peripheral sensory neuropathy (17 patients, 50.0%), back pain (16 patients, 47.1%), and gastroesophageal reflux disease (10 patients, 29.4%). Three patients (8.8%) had drug hypersensitivity reported in their medical history. Table 4 provides a summary of relevant respiratory medical histories, which included asthma in 5 patients (14.7%), bronchial hyperreactivity in 1 patient (2.9%) and chronic obstructive pulmonary disease in 2 patients (5.9%).

TABLE 4
Summary of relevant respiratory medical histories by system
organ class and preferred term in all treated patients.

| | Isatuximab |
|---|---|
| | (dose level and schedule) + |
| | pomalidomide/dexamethasone |
| Primary System Organ Class | 10 mg/kg QW/Q2W |
| Preferred Term n (%) | (N = 34) |
| Respiratory, thoracic and | 20 (58.8%) |
| mediastinal disorders | |
| Asthma | 5 (14.7%) |
| Bronchial hyperreactivity | 1 (2.9%) |
| Chronic obstructive | 2 (5.9%) |
| pulmonary disease | |
| Cough | 5 (14.7%) |
| Dyspnoea | 2 (5.9%) |
| Dyspnoea at rest | 1 (2.9%) |
| Dyspnoea exertional | 3 (8.8%) |
| Emphysema | 1 (2.9%) |
| Hypoxia | 2 (5.9%) |
| Lung infiltration | 1 (2.9%) |
| Oropharyngeal pain | 1 (2.9%) |
| Pleural effusion | 1 (2.9%) |
| Pleuritic pain | 1 (2.9%) |
| Pulmonary embolism | 2 (5.9%) |
| Pulmonary hypertension | 1 (2.9%) |
| Rhinitis allergic | 3 (8.8%) |
| Rhinorrhoea | 1 (2.9%) |
| Sleep apnoea syndrome | 6 (17.6%) |
| Wheezing | 1 (2.9%) |

As shown in Table 5, all patients had received an immunomodulatory agent (IMiD®), including lenalidomide, pomalidomide, or thalidomide; a proteasome inhibitor (PI), including bortezomib, carfilzomib, ixazomib citrate, marizomib, or oprozomib; and corticosteroids including dexamethasone or prednisone, in prior lines of treatment. Twenty-nine patients (85.3%) received an alkylating agent (bendamustine, carmustine, cyclophosphamide, melphalan or melphalan flufenamide) in prior lines of treatment. Fourteen (41.2%) and 7 (20.6%) had received prior pomalidomide and carfilzomib respectively. Prior to study entry, five (14.7%) and 7 (20.6%) patients had received daratumumab (anti-CD38 monoclonal antibody) and elotuzumab (anti-SLAM7 monoclonal antibody), respectively.

TABLE 5
Prior anti-cancer treatments in all treated patients.

| | Isatuximab (dose level |
|---|---|
| | and schedule) + |
| | pomalidomide/dexamethasone |
| | 10 mg/kg QW/Q2W |
| | (N = 34) |
| Main prior treatments[n(%)] (CDG) | |
| Alkylating agent | 29 (85.3%) |
| Bendamustine | 1 (2.9%) |
| Carmustine | 1 (2.9%) |
| Cyclophosphamide | 19 (55.9%) |
| Melphalan | 25 (73.5%) |
| Melphalan flufenamide | 1 (2.9%) |
| PI or IMiD ® agent | 34 (100%) |
| Lenalidomide or Bortezomib | 34 (100%) |
| Caifilzomib or Pomalidomide | 18 (52.9%) |
| PI and IMiD ® agent | 34 (100%) |
| Lenalidomide and Bortezomib | 32 (94.1%) |
| Caifilzomib and Pomalidomide | 3 (8.8%) |
| IMiD ® agent | 34 (100%) |
| Lenalidomide | 33 (97.1%) |
| Pomalidomide | 14 (41.2%) |
| Thalidomide | 7 (20.6%) |
| PI agent | 34 (100%) |
| Bortezomib | 33 (97.1%) |
| Caifilzomib | 7 (20.6%) |
| Ixazomib citrate | 8 (23.5%) |
| Marizomib | 1 (2.9%) |
| Oprozomib | 1 (2.9%) |
| moAb | 12 (35.3%) |
| Daratumumab | 5 (14.7%) |
| Elotuzumab | 7 (20.6%) |
| HDAC inhibitors | 1 (2.9%) |
| Panobinostat | 1 (2.9%) |
| Anthracyclines | 5 (14.7%) |
| Doxorubicin | 4 (11.8%) |
| Pegylated liposomal doxorubicin | 1 (2.9%) |
| hydrochloride | |
| Vinca alkaloids | 1 (2.9%) |
| Vincristine | 1 (2.9%) |
| Corticosteroids | 34 (100%) |
| Dexamethasone | 34 (100%) |
| Prednisone | 1 (2.9%) |

CDG:Customized Drug Grouping.

B. Extent of Isatuximab Exposure

Overall, the median number of isatuximab infusion cycles was 3.5 (min-max: 1 to 9) with 17 (50.0%) patients having started at least 4 cycles (minimum 9 infusions). The overall median duration of exposure was 13.4 weeks (min-max: 1 to 37). The median relative dose intensity (RDI) of isatuximab was 94.80% (69.8% to 112.9%) (Table 6). The median relative dose intensity of pomalidomide and dexamethasone were 84.7% and 87.5%, respectively

TABLE 6
Overall extent of exposure in all treated patients.

| | Isatuximab (dose | |
|---|---|---|
| | level and schedule) + | |
| | pomalidomide/dexamethasone | |
| | 10 mg/kg QW/Q2W | |
| | (N = 34) | |
| Total number of cycles started | 147 | |
| Number of cycles started by patient | | |
| Number | 34 | |
| Mean (SD) | 4.3 | (2.4) |
| Median | 3.5 | |
| Min:Max | 1:9 | |
| Number of cycles started | | |
| by patient [n(%)] | | |
| At least 1 | 34 | (100%) |
| At least 2 | 32 | (94.1%) |
| At least 3 | 27 | (79.4%) |
| At least 4 | 17 | (50.0%) |
| At least 6 | 11 | (32.4%) |
| At least 7 | 7 | (20.6%) |
| At least 8 | 6 | (17.6%) |
| At least 9 | 2 | (5.9%) |
| Duration of exposure (weeks) | | |
| Number | 34 | |
| Mean (SD) | 16.8 | (9.6) |
| Median | 13.4 | |
| Min:Max | 1:37 | |
| Isatuximab relative dose | | |
| intensity (%) | | |
| Number | 34 | |
| Mean (SD) | 93.62 | (9.87) |
| Median | 94.80 | |
| Min:Max | 69.8:112.9 | |
| Pomalidomide relative dose | | |
| intensity (%) | | |
| Number | 33 | |
| Mean (SD) | 82.89 | (16.30) |
| Median | 85.88 | |
| Min:Max | 49.4:100.0 | |
| Dexamethasone relative dose | | |
| intensity (%) | | |
| Number | 34 | |
| Mean (SD) | 85.52 | (17.00) |

| | |
|---|---|
| Median | 91.11 |
| Min:Max | 41.9:101.8 |

C. Dose Modifications and Withdrawal

As shown in Table 7, delay of isatuximab infusion (within cycle, excluding first infusion of cycle) occurred in 1 (3.0%) patient. Dose omissions occurred in 12 (35.3%) patients, with 16 of 146 (11.0%) cycles having one dose omission.

Seventeen (50.0%) patients had at least 1 infusion interruption, and overall, 17 of 317 (5.4%) infusions were temporarily interrupted before being completed. All the infusion interruptions occurred exclusively at the first infusion (Table 7). The median time from the start of infusion to the first interruption was 85 minutes (min-max: 46 to 145 minutes), with most of the interruptions occurring between 61 to 90 minutes (6 interruptions) (Table 7). There were no isatuximab dose reductions.

TABLE 7
Isatuximab dose modifications.

| | Isatuximab (dose level and schedule) + pomalidomide/ | |
|---|---|---|
| | dexamethasone 10 mg/kg QW/Q2W | |
| | (N = 34) | |
| Number of patients who could have an infusion delay^{a} | 33 | |
| Patients with at least 1 infusion delay [n(%)] | 1 | (3.0%) |
| Number of treated patients | 34 | |
| Patients with at least 1 dose omission [n(%)] | 12 | (35.3%) |
| Patients with at least 1 dose reduction [n(%)] | 0 | |
| Patients with at least 1 infusion interrupted | 17 | (50.0%) |
| [n(%)] | | |
| Patients with at least 1 infusion interrupted and | 17 | (50.0%) |
| re-started | | |
| Patients with at least 1 infusion interrupted and | 0 | |
| not re-started | | |
| Patients with at least 2 infusion interrupted | 0 | |
| [n(%)] | | |
| Number of isatuximab cycles | 146 | |
| Cycles with at least 1 dose omission [n(%)] | 16 | (11.0%) |
| Cycles with at least 1 dose reduction [n(%)] | 0 | |
| Cycles with at least 1 infusion interrupted [n(%)] | 17 | (11.6%) |
| Number of isatuximab infusions | 317 | |
| Isatuximab infusion interrupted [n(%)] | 17 | (5.4%) |
| Isatuximab infusion interrupted and re-started | 17 | (5.4%) |
| Isatuximab infusion interrupted and not re- | 0 | |
| started | | |
| Isatuximab infusion interrupted more than once | 0 | |
| [n(%)] | | |
| Infusions interrupted | 17 | |
| 1st infusion | 17 | (100%) |
| 2nd infusion | 0 | |
| 3rd infusion | 0 | |
| 4th infusion | 0 | |
| 5th infusion | 0 | |
| 6th infusion | 0 | |
| Subsequent infusions | 0 | |
| Time from infusion start to first interruption | | |
| (minutes) ^{b} | | |
| Number | 17 | |
| Mean (SD) | 88.8 | (31.3) |
| Median | 85.0 | |
| Min:Max | 46:145 | |
| <5 minutes | 0 | |
| 5-10 minutes | 0 | |
| 11-30 minutes | 0 | |
| 31-40 minutes | 0 | |
| 41-50 minutes | 2 | (11.8%) |
| 51-60 minutes | 2 | (11.8%) |
| 61-90 minutes | 6 | (35.3%) |
| 91-120 minutes | 3 | (17.6%) |
| >120 minutes | 4 | (23.5%) |

| | |
|---|---|
| Missing | 0 |

^{a}Patient was treated with at least two infusions.
^{b} For all infusions with at least one dose interruption.

As shown in Table 8, of the seventeen patients who had an isatuximab infusion interruption, 15 had the interruption because of a treatment emergent adverse event (TEAE). None of the TEAEs was Grade≥3. The remaining two (5.9%) patients who had infusion interruptions experienced food intolerance (G1 nausea and G1 vomiting) or technical issues, and their infusion interruptions were not related to IRs. In both cases the infusions were shortly interrupted, but were continued without decrease in infusion rate after restart and until completion as planned.

TABLE 8
Summary of treatment emergent adverse events leading
to isatuximab dose interruption by primary system
organ class (SOC) and preferred term (PT) presented by
all grades and grade ≥3.

| | Isatuximab (dose level and schedule) + | |
|---|---|---|
| | pomalidomide/dexamethasone | |
| | 10 mg/kg QW/Q2W | |
| Primary system organ class | (N = 34) | |
| Preferred term n(%) | All grades | Grade ≥3 |
| Any Event | 15 (44.1%) | 0 |
| Injury, poisoning and procedural | 15 (44.1%) | 0 |
| complications | | |
| Infusion related reaction | 15 (44.1%) | 0 |

Treatment-Emergent Adverse Events (TEAEs)

As shown in Table 9, the median durations of the first and second infusions were 3.94 hours (min-max: 3.3 to 6.1 hours) and 1.88 hours (min-max: 1.5 to 3.5 hours), respectively. Each of the third, fourth, fifth, and sixth infusions had a median duration of 1.27 hours. The median duration of the third and subsequent infusions, which were administered at a fixed infusion rate of 200 mL/hour, was 1.25 hours (min-max: 0.7 to 3.4 hours).

TABLE 9
Isatuximab duration of infusion in all treated patients.

| | Isatuximab (dose level and schedule) + | |
|---|---|---|
| | pomalidomide/dexamethasone | |
| | 10 mg/kg QW/Q2W | |
| Duration of infusion^{a} (hrs) | (N = 34) | |
| 1st infusion | | |
| Number | 34 | |
| Mean (SD) | 4.34 | (0.98) |
| Median | 3.94 | |
| Min:Max | 3.3:6.1 | |
| 2nd infusion | | |
| Number | 33 | |
| Mean (SD) | 2.06 | (0.54) |
| Median | 1.88 | |
| Mm:Max | 1.5:3.5 | |
| 3rd infusion | | |
| Number | 32 | |
| Mean (SD) | 1.45 | (0.42) |
| Median | 1.27 | |
| Min:Max | 1.2:3.4 | |
| 4th infusion | | |
| Number | 32 | |
| Mean (SD) | 1.35 | (0.17) |
| Median | 1.27 | |
| Min:Max | 1.1:2.0 | |
| 5th infusion | | |
| Number | 31 | |
| Mean (SD) | 1.30 | (0.11) |
| Median | 1.27 | |
| Min:Max | 1.1:1.6 | |
| 6th infusion | | |
| Number | 29 | |
| Mean (SD) | 1.33 | (0.13) |
| Median | 1.27 | |
| Mm:Max | 1.2:1.6 | |
| >3rd infusion | | |
| Number | 250 | |
| Mean (SD) | 1.33 | (0.20) |

| | |
|---|---|
| Median | 1.25 |
| Min:Max | 0.7:3.4 |

^{a}Duration of infusion was defined from the start time of infusion to the end time of infusion, including interruption time (if any).

E. Infusion Reactions

i. Infusion Reactions in All Treated Patients

Despite the short duration of infusions using a fixed volume, no Grade≥3 IRs were reported, and all IRs were Grade 2. There were no treatment discontinuations due to IRs. As shown in Table 10, IRs were reported in 16/34 (47.1%) patients, and in 16/317 (5.0%) infusions. All patients that experienced an IR developed only one episode of IR, and only during their first infusion of isatuximab. The onset of all the IRs occurred during the same day of the isatuximab infusion, and all the IRs recovered on the same day. IRs that occurred in >2 patients were dyspnea and cough (n=6 for each), and chills (n=3)

TABLE 10
Description of infusion reactions (generic term
as reported by investigator).

| | Isatuximab (dose level |
|---|---|
| | and schedule) + |
| | pomalidomide/dexamethasone |
| | 10 mg/kg QW/Q2W |
| | (N = 34) |
| ANALYSIS BY PATIENT | |
| Number of patients | 34 |
| Worst grade by patient [n(%)] | |
| All grades | 16 (47.1%) |
| Grade 1 | 0 |
| Grade 2 | 16 (47.1%) |
| Grade 3 | 0 |
| Grade 4 | 0 |
| Grade 5 | 0 |
| Action taken with isatuximab | |
| by patient [n(%)] | |
| Dose not changed | 1 (2.9%) |
| Dose delayed | 0 |
| Dose reduced | 0 |
| Dose delayed and reduced | 0 |
| Dose interrupted | 15 (44.1%) |
| Drug withdrawn | 0 |
| Not applicable | 0 |
| Corrective treatment given [n(%)] | 14 (41.2%) |
| Episodes by patient [n(%)] | |
| Only 1 episode | 16 (47.1%) |
| ≥1 episode | 16 (47.1%) |
| ≥2 episodes | 0 |
| ≥3 episodes | 0 |
| ≥4 episodes | 0 |
| ≥5 episodes | 0 |
| First occurrence of the IR at [n(%)] | |
| 1st Infusion | 16 (47.1%) |
| 2nd Infusion | 0 |
| 3rd Infusion | 0 |
| 4th Infusion | 0 |
| Subsequent infusions | 0 |
| Onset of IR leading to drug | |
| withdrawal at [n(%)] | |
| 1st Infusion | 0 |
| 2nd Infusion | 0 |
| 3rd Infusion | 0 |
| 4th Infusion | 0 |
| Subsequent infusions | 0 |
| Patient with IRs at [n(%)] | |
| 1st Infusion only | 16 (47.1%) |
| 1st Infusion | 16 (47.1%) |
| 2nd Infusion | 0 |
| 3rd Infusion | 0 |
| 4th Infusion | 0 |
| Subsequent infusions | 0 |
| Patients with at least 2 episodes of | 0 |
| IRs at the same infusion [n(%)] | |
| ANALYSIS BY INFUSION | |
| Number of infusions | 317 |
| Worst grade by infusion [n(%)] | |
| All grades | 16 (5.0%) |
| Grade1 | 0 |
| Grade2 | 16 (5.0%) |
| Grade3 | 0 |
| Grade4 | 0 |
| Grade5 | 0 |
| ANALYSIS BY EPISODE | |
| Number of episodes | 16 |
| Proportion of IRs occurring | |
| at [n(%)]: | |
| Infusion1 | 16 (100%) |
| Infusion 2 | 0 |
| Infusion 3 | 0 |
| Infusion 4 | 0 |
| Infusion 5 | 0 |
| Infusion 6 | 0 |
| Infusion >6 | 0 |
| IR duration [n(%)] | |
| 1 day | 16 (100%) |
| 2 days | 0 |
| >2 days | 0 |
| Not recovered | 0 |
| Day of onset of IR related to | |
| isatuximab administration | |
| Infusion day | 16 (100%) |
| 1 day after infusion | 0 |
| 2 days after infusion | 0 |
| 3 days after infusion | 0 |
| >3 days after infusion | 0 |

Infusion reactions were selected using Customized MedDRA queries (CMQ). An episode corresponded to a unique reference ID.

ii. Infusion Reactions Leading to Isatuximab Infusion Interruption

Fifteen of 16 patients with IRs had their isatuximab infusion interrupted. In the remaining patient, who experienced a Grade 2 IR and Grade 3 hypoxia (symptom of the IR), the isatuximab infusion was not interrupted, and the hypoxia was managed with supplementary oxygen administration. Symptoms of IRs that occurred in >1 patient were cough (6 patients, 17.6%), dyspnea (5 patients, 14.7%), nasal congestion (2 patients, 5.9%), and chills (2 patients, 5.9%). Most of the symptoms that were associated with IRs were reported as Grade 1 or 2, except for Grade 3 hypoxia and Grade 3 dyspnea (1 patient, 2.9% each) (Table 11).

TABLE 11
Summary of infusion reactions (including symptoms as
reported by investigator) by primary SOC and
PT presented by all grades and Grade ≥3.

| | Isatuximab (dose level | |
|---|---|---|
| | and schedule) + | |
| | pomalidomide/dexamethasone | |
| | 10 mg/kg QW/Q2W | |
| Primary system organ class | (N = 34) | |
| Preferred term n(%) | All grades | Grade ≥3 |
| Any event | 16 (47.1%) | 2 (5.9%) |
| Nervous system disorders | 1 (2.9%) | 0 |
| Paraesthesia | 1 (2.9%) | 0 |
| Vascular disorders | 1 (2.9%) | 0 |
| Flushing | 1 (2.9%) | 0 |
| Respiratory, thoracic and | 12 (35.3%) | 2 (5.9%) |
| mediastinal disorders | | |
| Dyspnoea | 5 (14.7%) | 1 (2.9%) |
| Cough | 6 (17.6%) | 0 |
| Nasal congestion | 2 (5.9%) | 0 |
| Oropharyngeal pain | 1 (2.9%) | 0 |
| Hypoxia | 1 (2.9%) | 1 (2.9%) |
| Sneezing | 1 (2.9%) | 0 |
| Throat irritation | 1 (2.9%) | 0 |
| Wheezing | 1 (2.9%) | 0 |
| Respiratory tract congestion | 1 (2.9%) | 0 |
| Gastrointestinal disorders | 1 (2.9%) | 0 |
| Nausea | 1 (2.9%) | 0 |
| Skin and subcutaneous | 1 (2.9%) | 0 |
| tissue disorders | | |
| Pruritus | 1 (2.9%) | 0 |
| General disorders and administration | 5 (14.7%) | 0 |
| site conditions | | |
| Non-cardiac chest pain | 1 (2.9%) | 0 |
| Chills | 2 (5.9%) | 0 |
| Chest discomfort | 1 (2.9%) | 0 |
| Chest pain | 1 (2.9%) | 0 |
| Injury, poisoning and procedural | 16 (47.1%) | 0 |
| complications | | |
| Infusion related reaction | 16 (47.1%) | 0 |

IRs were managed by dose interruption and/or use of medication consisting of H1/H2 blockers, and/or paracetamol, and/or montelukast, and/or steroids. H1/H2 blockers and steroids were used in 9/16 patients each (56.3%), paracetamol in 3/16 patients (18.8%), and montelukast in 1/16 patient (6.3%) (Table 12).

TABLE 12
Listing of IRs and symptoms according to investigator's reporting along with postmedications.

| | | | | H1/H2 | | | |
|---|---|---|---|---|---|---|---|
| Patient ID | Cycle/Day | IR term | IR grade | blockers | Paracetamol | Montelukast | Steroids |
| A | Cycle1/day 1 | Chills | 1 | Y | N | N | N |
| | Cycle1/day 1 | Infusion related | 2 | Y | N | N | N |
| | | reaction | | | | | |
| B | Cycle1/day 1 | Chest pain | 1 | N | Y | N | N |
| | Cycle1/day 1 | Dyspnoea | 1 | N | Y | N | N |
| | Cycle1/day 1 | Infusion related | 2 | N | Y | N | N |
| | | reaction | | | | | |
| | Cycle1/day 1 | Oropharyngeal | 1 | N | Y | N | N |
| | | pain | | | | | |
| C | Cycle1/day 1 | Dyspnoea | 2 | Y | N | Y | Y |
| | Cycle1/day 1 | Infusion related | 2 | Y | N | Y | Y |
| | | reaction | | | | | |
| D | Cycle1/day 1 | Cough | 2 | Y | N | N | Y |
| | Cycle1/day 1 | Infusion related | 2 | Y | N | N | Y |
| | | reaction | | | | | |
| | Cycle1/day 1 | Nasal congestion | 2 | Y | N | N | Y |
| | Cycle1/day 1 | Sneezing | 2 | Y | N | N | Y |
| E | Cycle1/day 1 | Dyspnoea | 2 | N | N | N | Y |
| | Cycle1/day 1 | Infusion related | 2 | N | N | N | Y |
| | | reaction | | | | | |
| F | Cycle1/day 1 | Infusion related | 2 | Y | N | N | Y |
| | | reaction | | | | | |
| | Cycle1/day 1 | Nausea | 2 | Y | N | N | Y |
| | Cycle1/day 1 | Non-cardiac | 2 | Y | N | N | Y |
| | | chest pain | | | | | |
| G | Cycle1/day 1 | Infusion related | 2 | Y | N | N | Y |
| | | reaction | | | | | |
| | Cycle1/day 1 | Nasal congestion | 2 | Y | N | N | Y |
| | Cycle1/day 1 | Wheezing | 2 | Y | N | N | Y |
| H | Cycle1/day 1 | Hypoxia | 3 | N | N | N | N |
| | Cycle1/day 1 | Infusion related | 2 | N | N | N | N |
| | | reaction | | | | | |
| I | Cycle1/day 1 | Chills | 2 | N | N | N | Y |
| | Cycle1/day 1 | Infusion related | 2 | N | N | N | Y |
| | | reaction | | | | | |
| J | Cycle1/day 1 | Infusion related | 2 | N | N | N | N |
| | | reaction | | | | | |
| | Cycle1/day 1 | Pruritus | 1 | N | N | N | N |
| K | Cycle1/day 1 | Cough | 1 | Y | Y | N | Y |
| | Cycle1/day 1 | Dyspnoea | 3 | Y | Y | N | Y |
| | Cycle1/day 1 | Infusion related | 2 | Y | Y | N | Y |
| | | reaction | | | | | |
| L | Cycle1/day 1 | Cough | 2 | Y | N | N | N |
| | Cycle1/day 1 | Infusion related | 2 | Y | N | N | N |
| | | reaction | | | | | |
| M | Cycle1/day 1 | Cough | 1 | N | N | N | N |
| | Cycle1/day 1 | Infusion related | 2 | N | N | N | N |
| | | reaction | | | | | |
| N | Cycle1/day 1 | Cough | 2 | Y | Y | N | Y |
| | Cycle1/day 1 | Infusion related | 2 | Y | Y | N | Y |
| | | reaction | | | | | |
| | Cycle1/day 1 | Respiratory tract | 2 | Y | Y | N | Y |
| | | congestion | | | | | |
| O | Cycle1/day 1 | Dyspnoea | 2 | N | N | N | N |
| | Cycle1/day 1 | Flushing | 2 | N | N | N | N |
| | Cycle1/day 1 | Infusion related | 2 | N | N | N | N |
| | | reaction | | | | | |
| P | Cycle1/day 1 | Chest discomfort | 2 | Y | N | N | Y |
| | Cycle1/day 1 | Cough | 2 | Y | N | N | Y |
| | Cycle1/day 1 | Infusion related | 2 | Y | N | N | Y |
| | | reaction | | | | | |
| | Cycle1/day 1 | Paraesthesia | 2 | Y | N | N | Y |
| | Cycle1/day 1 | Throat irritation | 2 | Y | N | N | Y |

As per safety complementary form, subjects 840-017-203 and 840-017-206 received H1/H2 blockers and steroids as medications for infusion reaction.

iii. Infusion Reactions in Patients with Relevant Therapeutic Histories and Respiratory Medical Histories

Of the 7 patients with prior exposure to daratumumab, 3 experienced IRs.

There was an acceptable tolerability of IRs in patients with a medical history of bronchial disorders (asthma, bronchial hyperreactivity, COPD).

Seven patients (20.6%) had a medical history of bronchospasm and obstruction (see Table 4). As shown in Table 13, five of these 7 patients experienced a Grade 2 IR. Most frequently, the IRs were managed with bronchodilators and steroids.

TABLE 13
Listing of IRs and their management in patients with
Bronchospasm and obstruction in medical history.

| Patient ID | Medical history | IR term | IR grade | Hydrocortisone | Motelukast | Methylprednisolone |
|---|---|---|---|---|---|---|
| C | Asthma | Dyspnoea | 2 | N | N | Y |
| | | Infusion | 2 | N | N | Y |
| | | related | | | | |
| | | reaction | | | | |
| E | Chronic | Dyspnoea | 2 | N | N | N |
| | obstructive | | | | | |
| | pulmonary | | | | | |
| | disease | | | | | |
| | | Infusion | 2 | N | N | N |
| | | related | | | | |
| | | reaction | | | | |
| K | Asthma | Cough | 1 | N | N | N |
| | | Dyspnoea | 3 | N | N | N |
| | | Infusion | 2 | N | N | N |
| | | related | | | | |
| | | reaction | | | | |
| M | Bronchial | Cough | 1 | N | N | N |
| | hyperreactivity | | | | | |
| | | Infusion | 2 | N | N | N |
| | | related | | | | |
| | | reaction | | | | |
| O | Asthma | Dyspnoea | 2 | N | N | N |
| | | Flushing | 2 | N | N | N |
| | | Infusion | 2 | N | N | N |
| | | related | | | | |
| | | reaction | | | | |
| | Chronic | Dyspnoea | 2 | N | N | N |
| | obstructive | | | | | |
| | pulmonary | | | | | |
| | disease | | | | | |
| | | Flushing | 2 | N | N | N |
| | | Infusion | 2 | N | N | N |
| | | related | | | | |
| | | reaction | | | | |

F. Immune Response

Anti-drug antibodies (ADA) against isatuximab are being assessed throughout the study in patient plasma using the P and A method in a 100 μl assay volume (Sanofi, Alfortville, France).

G. Efficacy

Efficacy was assessed according to the updated IMWG Response Criteria (Kumar S. et al, Lancet Oncol. 2016; 17(8):e328-e46 and Durie et al. (2006) “International uniform response criteria for multiple myeloma. Leukemia. 20: 1467-1473) to evaluate the percentage of patients with objective response (Overall Response Rate “ORR”), with Clinical Benefit Response (“CBR”) using IMWG defined response criteria, and duration of response (DOR).

Overall Response Rate: The ORR was defined as the proportion of patients with stringent complete response (sCR), complete response (CR), very good partial response (VGPR), and partial response (PR) using the updated IMWG Response Criteria (see Table 14). Response evaluation was performed on a monthly basis and included the following:

M-protein quantification (serum and 24-hr urine).

Serum free light chain levels.

Bone marrow biopsy/aspiration (if clinicallyindicated).

CT/MRI scan of plasmacytoma (if clinically indicated).

Bone skeletal survey (if clinically indicated).

Clinical Benefit Response: The CBR was defined as the proportion patients with sCR, CR, VGPR, PR and minimal response (MR) according to the IMWG criteria (see Table 14).

Duration of Response: The DOR was evaluated as the time from the date of the first response to the date of subsequent PD or death, whichever happened earlier. In the absence of the confirmation of subsequent disease progression or death before the end of the study, the DOR was censored at the date of the last valid assessment performed before the end of the study, or date of initiation of new anticancer treatment, whichever was earlier. DOR was determined only for patients who achieved a response of >PR. DOR was not calculated for patients that did not achieve a response.

TABLE 14
Standard International Myeloma Working Group (IMWG)
Response Criteria

| Response | IMWG Criteria |
|---|---|
| Complete Response | negative immunofixation on the serum and urine, |
| (CR) | and |
| | disappearance of any soft tissue plasmacytomas, |
| | and |
| | <5% plasma cells in bone marrow aspirates. |
| | A normal FLC ratio of 0.26-1.65 is required. |
| | Two consecutive assessments are needed. No known |
| | evidence of progressive disease or new bone marrow |
| | lesions if radiographic studies were performed |
| Stringent Complete | CR as defined above, plus: |
| Response (sCR) | a normal free light chain (FLC) ratio of 0.26-1.65, |
| | and |
| | absence of clonal cells in bone marrow by |
| | immunohistochemistry (κ/λ ratio ≤4:1 or ≥1:2 for κ |
| | and λ patients, respectively, after counting ≥100 |
| | plasma cells) |
| | Two consecutive assessments of laboratory |
| | parameters are needed. No known evidence of pro- |
| | gressive disease or new bone marrow lesions if |
| | radiographic studies were performed. |
| Very Good Partial | serum and urine M-protein detectable by |
| Response (VGPR) | immunofixation but not on electrophoresis, or |
| | ≥90% reduction in serum M-protein plus urine M- |
| | protein level <100 mg/24 h. |
| | Two consecutive assessments of laboratory |
| | parameters are needed. No known evidence of pro- |
| | gressive disease or new bone marrow lesions if |
| | radiographic studies were performed. |
| Partial Response | ≥50% reduction of serum M-protein and reduction in |
| (PR) | 24 hours urinary M-protein by ≥90% or to <200 |
| | mg/24 h, and |
| | If present at baseline, a ≥50% reduction in the size |
| | (SPD≠) of soft tissue plasmacytomas is also required. |
| | Two consecutive assessments of laboratory |
| | parameters are needed. No known evidence of |
| | progressive disease or new bone marrow lesions |
| | if radiographic studies were performed. |
| Minimal Response | 25% but <49% reduction of serum M-protein and |
| (MR) | reduction in 24 hours urinary M-protein by 50-80%, |
| | which still exceed 200 mg/24 h, and |
| | If present at baseline, a ≥50% reduction in the size |
| | (SPD) of soft tissue plasmacytomas is also required. |
| | No known evidence of progressive disease or |
| | new bone marrow lesions if radiographic studies |
| | were performed. |
| Stable Disease | Not meeting criteria for CR, VGPR, PR, MR |
| (SD) | or progressive disease. |
| | Two consecutive assessments are needed. No known |
| | evidence of progressive disease or new bone marrow |
| | lesions if radiographic studies were performed |
| Progressive | Any one or more of the following criteria: |
| Disease (PD) | Increase of ≥25% from lowest confirmed |
| | value in any one of the following criteria: |
| | Serum M-protein (the absolute increase must have |
| | been ≥0.5 g/dL). |
| | Serum M-protein increase ≥1 g/dL if the lowest M |
| | component was ≥5 g/dL. |
| | Urine M-component (the absolute increase must |
| | have been ≥200 mg/24 h). |
| | Appearance of new lesion(s), ≥50% increase from |
| | nadir in SPD≠ of >1 lesion, or ≥50% increase in the |
| | longest diameter of a previous lesion >1 cm in short |
| | axis; |
| | ≥50% increase in circulating plasma cells (minimum |
| | of 200 cells per μL) if this is the only measure of |
| | disease |
| | Two consecutive assessments are needed. No known |
| | evidence of progressive disease or new bone marrow |
| | lesions if radiographic studies were performed |

≠SPD, sum of the products of the maximal perpendicular diameters of measured lesions

Of the 31 patients who were evaluable for efficacy, the ORR was 64.5%, and the median PFS was 17.58 months (95% CI: 6.538 to not reached). Efficacy results were consistent with the results of clinical trial NCT02990338

### H. Exploratory Analyses

The correlation of clinical response and genomic profiling, multiple myeloma molecular subtype (using cytogenetics), and bone marrow CD38 mRNA levels in bone marrow and/or blood samples are analyzed. In addition, cytogenetic analysis was carried out on blood samples for immune genetic determinants (such as Fc polymorphisms, Human Leukocyte Antigen (HLA) and Killer Immunoglobulin-like Receptors (KIR), etc.), and correlation with clinical response are determined. Finally, the correlation of immunophenotype (such as B-cell, T-cell, and Natural Killer (NK)-cell subsets) in peripheral blood with parameters of clinical response are assessed.

### Conclusions

Isatuximab administered in a 250 ml fixed infusion volume with infusion rate measured in mL/hour had a manageable safety profile and considerably shorter infusion time compared to an infusion method consisting of a weight-based volume administered in mg/h. In general, the safety profile (including infusion reactions) of isatuximab administration with a simplified infusion method based on a fixed volume in mL/h was manageable and consistent with the safety profile of observed for other methods of isatuximab infusion (see, e.g., clinical trial NCT02990338), where the infusion rate was measured in mg/h.

The study met its primary endpoint with no IRs Grade≥3 observed. All IRs were grade 2, occurred during the first isatuximab infusion, and resolved the same day; no delayed onset IRs were reported. The median infusion time for isatuximab 10 mg/kg administered in a 250 mL fixed infusion volume with an infusion rate in mL/h for the third and subsequent infusions was 75 minutes. This is considerably shorter than the infusion time for isatuximab administered as mg/h (median 174 minutes for third and subsequent infusions. The general safety profile of the simplified infusion of Isa was favorable and consistent with previous observations for this combination of Isa-Pd.

### Example 1B

### Further Results from a Phase 1b Study to Evaluate the Feasibility and Safety of Isatuximab Short Duration Fixed Volume Infusion in Combination with Pomalidomide and Dexamethasone for Relapsed and/or Refractory Multiple Myeloma

Further results from the Phase 1b study described in Example 1A are described below. Briefly, 47 patients were treated. Patient baseline characteristics are shown in Table 15 below. All patients had previously received lenalidomide and 48.9% had prior pomalidomide treatment. Prior daratumumab (Dara) exposure was recorded in 14.9% of patients and prior elotuzumab exposure in 19.1%. At study entry, the median length of time since initial diagnosis was about 6.2 years (range 1.1-22.7 years). 41 patients (87.2%) were refractory to their last regimen.

TABLE 15
Patient demographics and disease characteristics

| | All patients |
|---|---|
| Characteristic | (n = 47) |
| Median age, years (range) | 65 (45-85) |
| ISS stage at study entry, n (%) | |
| I | 23 (48.9) |
| II | 12 (25.5) |
| III | 7 (1 4.9) |
| Unknown | 5 (10.6) |
| Respiratory disorders at | |
| baseline, n % | |
| Asthma | 8 (17.0) |
| Bronchial hyperreactivity | 1 (2.1) |
| COPD | 2 (4.3) |
| Prior treatment and | |
| refractory status | |
| Prior treatments, n (%) | 47 (100) |
| Lenalidomide | 23 (48.9) |
| Pomalidomide | 46 (97.9) |
| Bortezomib | 7 (14.9) |
| Daratumumab | 9 (19.1) |
| Elotuzumab | |
| Refractory to, n (%) | 41 (87.2) |
| Lenalidomide | 23 (48.9) |
| Pomalidomide | 26 (55.3) |
| Bortezomib | 46 (97.9) |
| IMiD or PI | 35 (74.5) |
| IMiD and PI | |

COPD, chronic obstructive pulmonary disease;
IMID, Immunomodulatory drug;
ISS, International Staging System;
PI, proteasome Inhibitor

At the time of data cut-off, 30 (63.8%) patients remained on treatment and 17 (36.2%) had discontinued treatment. The reasons for discontinuations were disease progression (n=10), adverse event (AE; n=4), and other (n=3).

Treatment Exposure

The Median number of cycles was 4.0 with 45 (95.7%) patients having started at least 2 cycles (minimum 5 infusions) and 31 (66.0%) having started at least 4 cycles (minimum 9 infusions). Overall median duration of exposure was 18.1 weeks (range 1-45). The Median relative dose intensities for isatuximab, pomalidomide, and dexamethasone were 94.1%, 84.7% and 87.5%, respectively.

Of a total of 490 Isa infusions, 22 (4.5%) were interrupted and restarted. Twenty (90.9%) of the interruptions occurred on the first infusion. Twenty-five patients (53.2%) had ≥1 pomalidomide dose omission; 21 patients (44.7%) had ≥1 dose reduction. Most dose reductions (85.7%) occurred during Cycle 2.

Infusion Reactions

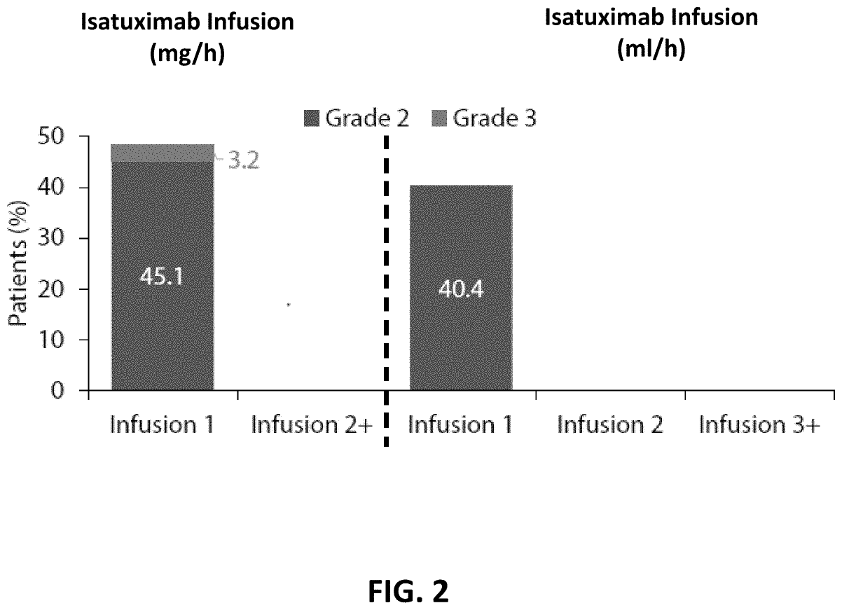

There were no grade≥3 IRs or treatment discontinuations due to IRs. IRs of any grade were reported in 19/47 (40.4%) patients and 19/490 (3.9%) infusions. In Part 1, 48.3% of patients who received Isa 10 mg/kg experienced an IR (FIG. 2). All IRs were Grade 2 severity, all occurred during the first infusion of Isa, and all the IRs recovered on the same day. IRs that occurred in >2 patients were dyspnea and cough (n=6 for each), and chills (n=3). IRs were managed with dose interruption in 18 (38.3%) patients, while the dose was not interrupted in 1 patient (2.1%).

In a parallel clinical trial designed to assess the efficacy of isatuximab+pomalidomide+dexamethasone in patients who had undergone at least two prior therapies for multiple myeloma, isatuximab was administered to study participants (n=31) according to a standard protocol, in which the isatuximab infusion rate was measured in mg/hour. The left side of FIG. 2 shows the number of patients from the parallel clinical trial who experienced IRs (i.e., who were administered with isatuximab according to the standard protocol). The right side of FIG. 2 shows the number of patients in the present clinical study who experienced IRs when administered with isatuximab in a 250 ml fixed volume (i.e., as described in Section III G in Example 1A). As discussed in Example 1A, isatuximab infusion rate was measured in in ml/h. The percentage of patients who experienced Grade≥2 IRs during the first infusion in the present study was 40.4%. By contrast, the percentage of patients in the parallel study who experienced Grade≥2 IRs (i.e., when administered with isatuximab according to the standard protocol) was 48.3%. None of the patients in the present feasibility and safety study experienced Grade 3 IRs during the first infusion. By contrast, 3.2% of the patients in the parallel study (i.e., who were administered with isatuximab according to the standard protocol) experienced Grade 3 IRs during the first infusion.

All IRs experienced by patients in the present study were managed with dose interruption, and, if justified, the use of medication consisting of H1/H2 blockers, and/or paracetamol, and/or montelukast, and/or steroids, and/or bronchodilators. The onset of all the IRs occurred during the same day of the Isa infusion (i.e., no delayed IRs occurred). Post-infusion prophylaxis was not needed. Of the 7 patients in the present study with prior exposure to Dara, 3 experienced IRs. There was an acceptable tolerability of IRs in patients in the present study with a medical history of bronchial disorders (asthma, bronchial hyperreactivity, COPD).

Infusion Duration

The left side of FIG. 2 shows the median durations of the first isatuximab infusion and the median durations of subsequent isatuximab infusions (i.e., following the first infusion) from the parallel clinical trial, i.e., in which participants were administered with isatuximab according to the standard protocol. The right side of FIG. 2 shows the median durations of the first isatuximab infusion, the second isatuximab infusion, and subsequent isatuximab infusions (i.e., following the second infusion) from the present study, i.e., in which participants were administered with isatuximab in a 250 ml fixed volume, as described in Section III G in Example 1A.

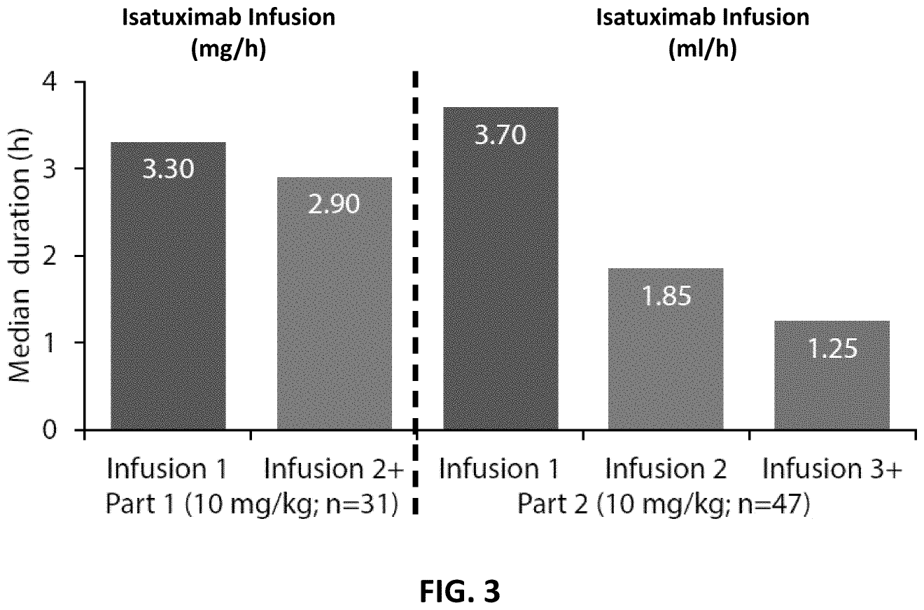

In the present study (which is alternatively referred to herein as “Part B”), the median duration of isatuximab infusion decreased from 3.70 h (222 mins, range: 1.0-6.1 hours) for first infusion to 1.85 h for the second infusion, and then further decreased to 1.25 h (75 mins) for third infusion onwards (right side of FIG. 3). Median infusion duration remained stable over subsequent infusions (1.25 h, range: 0.9-3.4 hours). Median infusion durations were 3.30 hours for the first infusion and 2.9 h (174 mins) for second infusion onwards in a parallel study, alternatively referred to herein as “Part A” (left side of FIG. 3), i.e., in which patients were administered with isatuximab according to the standard protocol (where infusion rate was measured as mg/hr).

Treatment-Emergent Adverse Events (TEAEs)

All but one patient (97.9%) experienced a TEAE with the most common being fatigue (55.3%), IRs (40.4%), and upper respiratory tract infections (38.3%), and neutropenia (38.3%). See, e.g., Table 16. Grade≥3 TEAEs were observed in 31 (68.1%) patients. Non-hematologic TEAEs observed in >2 patients included arthralgia, pneumonia and musculoskeletal pain (n=3 for each). Infections of any grade were reported in 34 (72.3%) patients, with Grade≥3 infections reported in 9 (19.1%) patients. Treatment-related TEAEs were experienced by 45 (95.7%) patients, with 26 (55.3%) experiencing treatment-related TEAEs of grade≥3. Serious TEAEs were observed in 23 (48.9%) patients, which were treatment-related in 12 (25.5%) patients. Four (8.5%) patients discontinued due to TEAEs (2 severe infections; 1 acute myocardial infarction; 1 sudden death). Six patients died during treatment period (≤30 days from last dose of study drug); there were no deaths during the post-treatment period (>30 days from last dose of study drug). Deaths were attributed to AE (n=3; acute myocardial infarction, sepsis, and rectal hemorrhage/sepsis), progressive disease (n=2), and 1 sudden death with unknown cause. No deaths were considered treatment-related.

TABLE 16
Most Common TEAEs

| TEAE*, n (%) | All grades | Grade ≥3 |
|---|---|---|
| Any | 46 (97.9) | 32 (68.1) |
| Fatigue | 26 (55.3) | 2 (4.3) |
| Infusion reactions | 19 (40.4) | 0 |
| Upper respiratory tract | 18 (38.3) | 2 (4.3) |
| infection | | |
| Dyspnea | 14 (29.8) | 1 (2.1) |
| Constipation | 13 (27.7) | 0 |
| Diarrhea | 13 (27.7) | 1 (2.1) |
| Nausea | 12 (25.5) | 0 |
| Cough | 11 (23.4) | 0 |
| Arthralgia | 10 (21.3) | 3 (6.4) |
| Pneumonia | 7 (14.9) | 3 (6.4) |
| Musculoskeletal pain | 5 (10.6) | 3 (6.4) |

*TEAE, treatment-emergent adverse event

Hematologic abnormalities (all grades) were observed in the majority of patients: leukopenia (95.7%); neutropenia (93.5%); anemia and thrombocytopenia (both 82.6%); lymphopenia (63.0%). Neutropenia was the most common Grade 3/4 hematologic adverse event with 17 patients experiencing Grade 3 and 16 patients Grade 4 (see Table 17). Grade 4 neutropenia was observed in 34.8% of patients and grade 4 thrombocytopenia in 8.7% of patients. 20 patients (42.6%) received granulocyte colony-stimulating factor.

TABLE 17
Grade 3/4 hematologic events (n = 46)

| Hematologic | | |
|---|---|---|
| abnormality, n (%) | Grade 3 | Grade 4 |
| Leukopenia | 25 (54.3) | 6 (13.0) |
| Neutropenia | 17 (37.0) | 16 (34.8) |
| Lymphopenia | 16 (34.8) | 4 (8.7) |
| Thrombocytopenia | 9 (19.6) | 4 (8.7) |
| Anemia | 8 (17.4) | 0 |

Conclusions

The study met its primary endpoint with no IRs Grade≥3 observed. All IRs were grade 2, occurred during the first isatuximab infusion, and resolved the same day; no delayed onset IRs were reported. The median infusion time for isatuximab 10 mg/kg administered in a 250 mL fixed infusion volume with an infusion rate in mL/h for the third and subsequent infusions was 75 minutes. This is considerably shorter than the infusion time from a parallel study in which isatuximab administered as mg/hr according to a standard administration protocol (median 174 minutes for third and subsequent infusions). The general safety profile of the simplified infusion of isatuximab was favorable and consistent with previous observations for this combination of isatuximab+pomalidomide+dexamethasone. A fixed infusion volume (250 ml) of isatuximab can be helpful with the monitoring of fluid balance recommended for patients with renal impairment. Additionally, the change from a weight-based volume administration method (mg/hr) for Isatuximab infusion to a fixed volume infusion method (ml/hr) had a limited impact on pharmacokinetic parameters with comparable simulated Cₘₐₓ at steady state (283 μml vs. 284 μg/ml) and C_{trough} (119 μg/ml vs. 119 μg/ml).

### Example 1C

### Efficacy Results from a Phase 1b Study to Evaluate the Feasibility and Safety of Isatuximab Short Duration Fixed Volume Infusion in Combination with Pomalidomide and Dexamethasone for Relapsed and/or Refractory Multiple Myeloma

Further results from the Phase 1b study described in Examples 1A and 1B are described below. 47 patients were included in the all-treated population. At the time of the final cut-off, 22 patients (46.8%) were still on treatment. The reasons for study treatment discontinuation at the time of the analysis were: disease progression (15 patients, 31.9%), adverse events (AEs); (5 patients, 10.6%), and other reasons (5 patients, 10.6%). One patient (2.1%) prematurely discontinued pomalidomide treatment due to an adverse event, and no patients prematurely discontinued dexamethasone treatment. See Table 17B.

TABLE 17B
Reasons for treatment discontinuation

| | Isatuximab (dose level |
|---|---|
| | and schedule) + |
| | pomalidomide/dexamethasone |
| | 10 mg/kg QW/Q2W |
| | (N = 47) |
| Off treatment | 25 (53.2%) |
| Reasons for definitive | |
| treatment | |
| discontinuation | |
| Adverse event | 5 (10.6%) |
| Disease progression | 15 (31.9%) |
| Other | 5 (10.6%) |
| Reasons for premature | |
| discontinuation of | |
| pomalidomide | |
| Adverse event | 1 (2.1%) |
| Ongoing treatment | 22 (46.8%) |

Demographics

The median age was 65 years (range 45 to 85 years), with the largest proportion of patients being aged <65 years (23 patients, 48.9%). All the patients had an ECOG PS of 0 or 1, except 2 patients (4.3%) who had an ECOG PS of 2. At baseline, patients had a body weight ranging from 40 kg to 121 kg with a median of 90.3 kg.

Medical History

The most frequent conditions reported in medical history consisted of: peripheral sensory neuropathy (27 patients, 57.4%); hypertension (25 patients, 53.2%); back pain (18 patients, 38.3%); and gastroesophageal reflux disease (15 patients, 31.9%). Relevant respiratory medical history included asthma in 8 patients (17.0%), and chronic obstructive pulmonary disease in 2 patients (4.3%).

Disease Characteristics at Study Entry

At study entry, 23 (48.9%), 12 (25.5%), and 7 (14.9%) patients had International Staging System (ISS) criteria of Stage I, II, and III, respectively. The ISS stage of 5 patients (10.6%) was unknown. At study entry, most patients (33 patients, 70.2%) had measurable serum M-protein.) criteria of Stage I, II, and III, respectively. The ISS stage of 5 patients (10.6%) was unknown. At study entry, most patients (33 patients, 70.2%) had measurable serum M-protein.

Patients had a median of 22.5% (range 0 to 100%) plasma cells in bone marrow. Most patients (33 patients, 70.2%) had bone lesions at baseline, and 12 (25.5%) patients had plasmacytoma present at baseline. There were 10 patients (21.3%) with high risk cytogenetic characteristics: cytogenetic abnormalities included del17p in 7 patients (14.9%), t(4;14) translocation in 3 patients (6.4%) and t(14;16) translocation in 1 patient (2.1%). Seventeen patients (36.2%) entered the study with moderate renal impairment (GFR 30 to ≤60 mL/min/1.73 m²) and one patient entered the study with severe renal impairment (GFR 15 to <30 mL/min/1.73 m²).

Prior Anti-Cancer Treatments

The median number of prior treatment lines was 3 (min-max: 1-8) with 1 patient (2.1%) having received 1 prior line of treatment and 17 patients (36.2%) having received 2 prior lines of treatment.

All patients had received an IMiD (including lenalidomide, pomalidomide, or thalidomide), PI agent (including bortezomib, carfilzomib, ixazomib, marizomib, or oprozomib) and corticosteroid (dexamethasone or prednisone) in prior lines of treatment. All patients had received prior lenalidomide. 19 patients (40.4%) were refractory to lenalidomide at last regimen prior to study entry. The majority (39 patients, 83.0%) of patients received an alkylating agent (bendamustine, carmustine, cyclophosphamide, melphalan or melphalan flufenamide) in prior lines. Twenty-three (48.9%) and 11 (23.4%) patients had received prior pomalidomide and carfilzomib respectively. Seven (14.9%) and 9 (19.1%) patients, respectively, had received daratumumab (anti-CD38 monoclonal antibody) and elotuzumab (anti-SLAM7 monoclonal antibody), prior to study entry.

### Efficacy

Overall Response Rate (ORR)

The ORR, determined in the all treated population (n=47), was 53.2% (95% confidence interval [CI]: 38.1% to 67.9%), including 2 patients (4.3%) with CR, 11 patients (23.4%) with VGPR, and 12 patients (25.5%) with PR. See Table 17C. The “at least VGPR” rate was 27.6%. The clinical benefit rate or “CBR” (MR or better) was 72.3% (95% CI: 57.4% to 84.4%), including all the above plus 9 patients (19.1%) with MR. Responses and disease progression were assessed by investigator. The “at least VGPR” rate was 27.6%. The clinical benefit rate or “CBR” (MR or better) was 72.3% (95% CI: 57.4% to 84.4%), including all the above plus 9 patients (19.1%) with MR. Responses and disease progression were assessed by investigator.

TABLE 17C
Best overall response, overall response rate
and clinical benefit rate

| | Isatuximab (dose level |
|---|---|
| | and schedule) + |
| | pomalidomide/ |
| | dexamethasone |
| | 10 mg/kg QW/Q2W |
| | (N = 47) |
| Overall Response Rate (≥PR) | 25 (53.2%) |
| 95% CI^{a} | (38.1% to 67.9%) |
| Complete response (CR) | 2 (4.3%) |
| Very Good Partial Response | 11 (23.4%) |
| (VGPR) | |
| Partial response (PR) | 12 (25.5%) |
| Minimal response (MR) | 9 (19.1%) |
| Stable disease (SD) | 11 (23.4%) |
| Not evaluable | 2 (4.3%) |
| Clinical benefit rate (≥MR) | 34 (72.3%) |
| 95% CI^{a} | (57.4% to 84.4%) |

^{a}estimated by Clopper-Pearson Exact method
CI:Confidence interval,
CR:Complete Response,
VGPR:Very Good Partial Response,
PR:Partial Response,
MR:Minimal response,
SD:stable disease

Among the 7 patients who had previous exposure to daratumumab treatment (see Table 17D), there was 1 response of PR for an ORR of 14.3%. In addition, 2 of the 7 patients (28.6%) obtained MR, resulting in a CBR of 42.9%. One patient with prior exposure to daratumumab was non-evaluable for response. The ORR for patients without prior daratumumab was 60.0% (24 of 40).

TABLE 17D
Best overall response, overall response rate and clinical benefit rate
in patients who received Daratumumab as prior therapy

| | Isatuximab (dose level and schedule) + |
|---|---|
| | pomalidomide/dexamethasone |
| | 10 mg/kg QW/Q2W |
| | (N = 7) |
| Overall Response Rate (≥PR) | 1 (14.3%) |
| 95% CI^{a} | (0.4% to 57.9%) |
| Partial response (PR) | 1 (14.3%) |
| Minimal response (MR) | 2 (28.6%) |
| Stable disease (SD) | 3 (42.9%) |
| Not evaluable | 1 (14.3%) |
| Clinical benefit rate (≥MR) | 3 (42.9%) |
| 95% CI^{a} | (9.9% to 81.6%) |

^{a}estimated by Clopper-Pearson Exact method
CI: Confidence interval, CR: Complete Response, VGPR: Very Good Partial Response, PR: Partial Response, MR: Minimal response, SD: stable disease

The ORRs was 52.2% (12 of 23) for patients with prior pomalidomide, 56.5% (13 of 23) for patients without prior pomalidomide or prior daratumumab, 54.5% (18 of 33) for patients with measurable M-protein on serum, 33.3% (2 of 6) for patients with measurable M-protein on urine, and 66.7% (4 of 6) for patients with disease measurable on FLC only

Duration of Follow-Up, Time to First Response, and Duration of Response (DOR)

The median duration of follow-up was 9.9 months (range: 0 to 17.3). The median time to first response was 0.95 months (range: 0.9 to 3.4).

Duration of response (DOR) was assessed using Kaplan-Meier method in 25 responders. All responding patients who had an ongoing response at the DOR analysis were censored (N=21) at last disease assessment. The median DOR and the 25^{th} quantile have not been reached.

### Progression Free Survival (PFS)

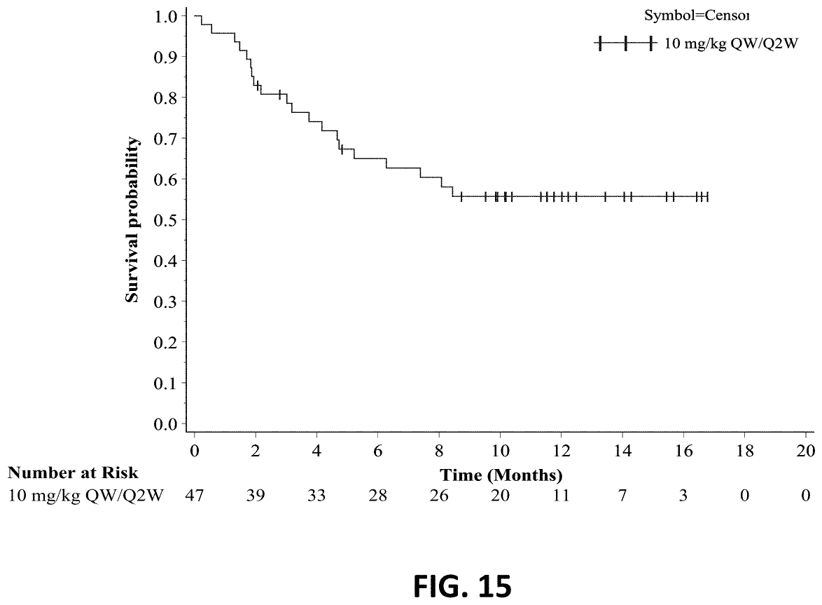

At the time of analysis, 20 patients (42.6%) were reported to have had a PFS event (i.e., confirmed progressive disease (PD), symptomatic deterioration or death), and 27 patients (57.4%) were censored. The median PFS has not been reached; the 6-month probability of PFS was 65.0% (95% CI: 49.3% to 76.9%) and the 12-month probability was 55.7% (95% CI: 40.1% to 68.8%). A Kaplan-Meier plot of PFS is provided in FIG. 15.

Overall Survival (OS)

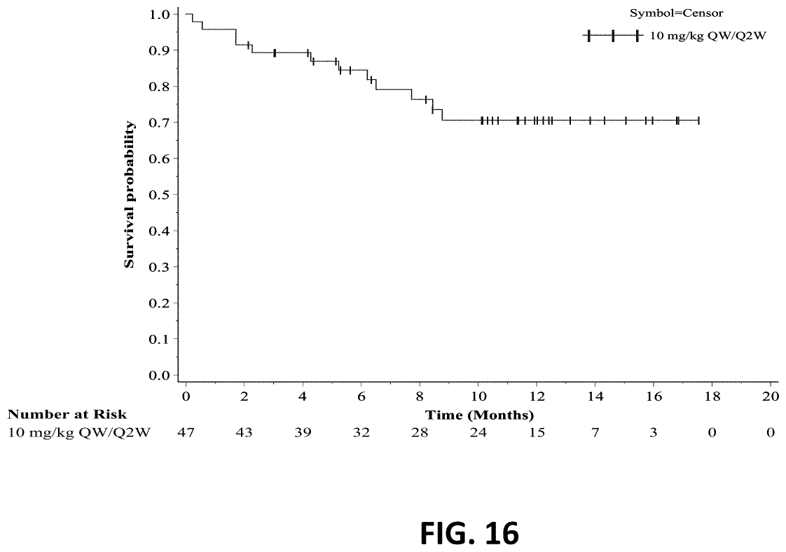

At the time of analysis, 12 patients (25.5%) were reported to have died. The median OS has not been reached. The probability of surviving 6 months was 84.5% (95% CI: 70.1% to 92.3%) and the probability of surviving 12 months was 70.6% (95% CI: 53.7% to 82.3%). A Kaplan-Meier plot of OS is provided in FIG. 16.

### Safety

Extent of Exposure

Overall, the median number of cycles was 9 (range: 1 to 19) with 31 (66.0%) patients having started at least 6 cycles and 18 (38.3%) patients having started at least 12 cycles. The overall median duration of exposure was 36.9 weeks (range 1 to 77). See Table 17E.

TABLE 17E
Overall extent of exposure

| | Isatuximab (dose level and schedule) + |
|---|---|
| | pomalidomide/dexamethasone |
| | 10 mg/kg QW/Q2W |
| | (N = 47) |
| Total number of cycles started | 425 |
| Number of cycles started by patient | |
| Number | 47 |
| Mean (SD) | 9.0 (5.5) |
| Median | 9.0 |
| Min:Max | 1:19 |
| Number of cycles | |
| started by patient[n(%)] | |
| At least 1 | 47 (100%) |
| At least 2 | 45 (95.7%) |
| At least 3 | 41 (87.2%) |
| At least 4 | 35 (74.5%) |
| At least 5 | 32 (68.1%) |
| At least 6 | 31 (66.0%) |
| At least 8 | 27 (57.4%) |
| At least 9 | 25 (53.2%) |
| At least 10 | 23 (48.9%) |
| At least 11 | 21 (44.7%) |
| At least 12 | 18 (38.3%) |
| At least 13 | 16 (34.0%) |
| At least 15 | 10 (21.3%) |
| At least 16 | 6 (12.8%) |
| At least 17 | 5 (10.6%) |
| At least 18 | 4 (8.5%) |
| At least 19 | 2 (4.3%) |
| Duration of exposure(weeks) | |
| Number | 47 |
| Mean (SD) | 37.2 (23.2) |
| Median | 36.9 |
| Min:Max | 1:77 |

Infusion duration is summarized in Table 17F. The median duration of the first infusion was 3.70 hours (range 1 to 6.1 hours); mediation duration of second infusion was 1.85 hours (range 1.5 to 3.9 hours); and from the third infusion onward the median duration was 1.25 hours (range 0.8 to 3.4 hours).

TABLE 17F
Isatuximab Duration of Infusion

| | Isatuximab (dose level and schedule) + |
|---|---|
| | pomalidomide/dexamethasone |
| | 10 mg/kg QW/Q2W |
| Duration of infusion^{a} (hrs) | (N = 47) |
| 1st infusion | |
| Number | 47 |
| Mean (SD) | 4.10 (1.06) |
| Median | 3.70 |
| Min:Max | 1.0:6.1 |
| 2nd infusion | |
| Number | 46 |
| Mean (SD) | 2.06 (0.55) |
| Median | 1.85 |
| Min:Max | 1.5:3.9 |
| 3rd infusion | |
| Number | 45 |
| Mean (SD) | 1.42 (0.39) |
| Median | 1.25 |
| Min:Max | 1.2:3.4 |
| 4th infusion | |
| Number | 45 |
| Mean (SD) | 1.33 (0.16) |
| Median | 1.25 |
| Min:Max | 1.1:2.0 |
| 5th infusion | |
| Number | 44 |
| Mean (SD) | 1.29 (0.09) |
| Median | 1.25 |
| Min:Max | 1.1:1.6 |
| 6th infusion | |
| Number | 42 |
| Mean (SD) | 1.31 (0.12) |
| Median | 1.25 |
| Min:Max | 1.2:1.6 |
| ≥3rd infusion | |
| Number | 777 |
| Mean (SD) | 1.32 (0.16) |
| Median | 1.25 |
| Min:Max | 0.8:3.4 |

^{a}Duration of infusion is defined from the start time of infusion to the end time of infusion including interruption time (if any).

Infusion Reactions

Infusion reactions (IRs) are summarized in Table 17G. IRs of any grade were reported in 19 patients (40.4%), and in 20 episodes in 871 infusions (2.3%). All the IRs were Grade 2 and no patient had IR of Grade≥3. All but 1 of the patients who experienced IRs had only a single episode, and all only during their first infusion of isatuximab; one patient (2.1%) had 2 IR episodes during the first infusion. The onset of all the IRs occurred during the same day of the isatuximab infusion, and all the IRs recovered on the same day.

IRs were managed with dose interruption and/or use of medication consisting of either H1/H2 blockers, and/or paracetamol, and/or montelukast, and/or steroids. Eighteen of 19 patients with IRs had their isatuximab infusion interrupted; in the remaining patient with a Grade 2 IR and Grade 3 hypoxia (symptom of the IR), the infusion was not interrupted, and the hypoxia was managed with oxygen administration.

Among the7 patients who had had previous exposure to daratumumab treatment, 3 experienced an IR.

TABLE 17G
Description of infusion
reactions (generic term as reported by investigator)

| | Isatuximab (dose level and |
|---|---|
| | schedule) + pomalidomide/ |
| | dexamethasone |
| | 10 mg/kg QW/Q2W |
| | (N = 47) |
| ANALYSIS BY PATIENT | |
| Number of patients | 47 |
| Worst grade by patient [n(%)] | |
| All grades | 19 (40.4%) |
| Grade 1 | 0 |
| Grade 2 | 19 (40.4%) |
| Grade 3 | 0 |
| Grade 4 | 0 |
| Grade 5 | 0 |
| Action taken with | |
| isatuximab by patient [n(%)] | |
| Dose not changed | 1 (2.1%) |
| Dose delayed | 0 |
| Dose reduced | 0 |
| Dose delayed and reduced | 0 |
| Dose interrupted | 18 (38.3%) |
| Drug withdrawn | 0 |
| Not applicable | 0 |
| Corrective treatment given [n(%)] | 18 (38.3%) |
| Episodes by patient [n(%)] | |
| Only 1 episode | 18 (38.3%) |
| ≥1 episode | 19 (40.4%) |
| ≥2 episodes | 1 (2.1%) |
| ≥3 episodes | 0 |
| ≥4 episodes | 0 |
| ≥5 episodes | 0 |
| First occurrence of the IR at [n(%)] | |
| 1st Infusion | 19 (40.4%) |
| 2nd Infusion | 0 |
| 3rd Infusion | 0 |
| 4th Infusion | 0 |
| Subsequent infusions | 0 |
| Onset of IR leading to drug withdrawal at | |
| [n(%)] | |
| 1st Infusion | 0 |
| 2nd Infusion | 0 |
| 3rd Infusion | 0 |
| 4th Infusion | 0 |
| Subsequent infusions | 0 |
| Patient with IRs at [n(%)] | |
| 1st Infusion only | 19 (40.4%) |
| 1st Infusion | 19 (40.4%) |
| 2nd Infusion | 0 |
| 3rd Infusion | 0 |
| 4th Infusion | 0 |
| Subsequent infusions | 0 |
| Patients with at least 2 episodes of | 1 (2.1%) |
| IRs at the same infusion [n(%)] | |
| ANALYSIS BY INFUSION | |
| Number of infusions | 871 |
| Number of episodes of IRs | 20 (2.3%) |
| Worst grade by infusion [n(%)] | |
| All grades | 19 (2.2%) |
| Grade 1 | 0 |
| Grade 2 | 19 (2.2%) |
| Grade 3 | 0 |
| Grade 4 | 0 |
| Grade 5 | 0 |
| ANALYSIS BY EPISODE | |
| Number of episodes | 20 |
| Proportion of IRs occurring at [n(%)]: | |
| Infusion 1 | 20 (100%) |
| Infusion 2 | 0 |
| Infusion 3 | 0 |
| Infusion 4 | 0 |
| Infusion 5 | 0 |
| Infusion 6 | 0 |
| Infusion >6 | 0 |
| IR duration [n(%)] | |
| 1 day | 20 (100%) |
| 2 days | 0 |
| >2 days | 0 |
| Not recovered | 0 |
| Day of onset of IR related to isatuximab | |
| administration | |
| Infusion day | 20 (100%) |
| 1 day after infusion | 0 |
| 2 days after infusion | 0 |
| 3 days after infusion | 0 |
| >3 days after infusion | 0 |

### Treatment-Emergent Adverse Events

All patients had at least 1 TEAE (any grade), 35 patients (74.5%) had Grade≥3 TEAEs regardless of relationship to study treatment, and 27 patients (57.4%) had at least one serious TEAE regardless of relationship to study treatment. There were 6 patients (12.8%) who experienced TEAEs leading to death during the study. Five (10.6) patients experienced TEAEs leading to definitive treatment discontinuation (i.e., discontinuation of all study treatment), in addition 1 patient (2.1%) experienced TEAEs leading to premature discontinuation of pomalidomide.

The most frequently reported non-hematologic TEAEs of any grade and regardless of relationship with study treatment (in >20% of patients) were fatigue (30 patients, 63.8%), infusion related reaction (19 patients, 40.4%), upper respiratory tract infection (19 patients, 40.4%), cough (19 patients, 40.4%), diarrhea (16 patients, 34.0%), nausea (16 patients, 34.0%), dyspnea (16 patients, 34.0%), insomnia (15 patients, 31.9%), back pain (14 patients, 29.8%), constipation (14 patients, 29.8%), arthralgia (13 patients, 27.7%), peripheral sensory neuropathy (10 patients, 21.3%), and pneumonia (10 patients, 21.3%). The most frequently reported Grade≥3 non-hematologic TEAEs (in >5% of patients) were pneumonia (5 patients, 10.6%), arthralgia (3 patients, 6.4%), upper respiratory tract infection (3 patients, 6.4%) and musculoskeletal pain (3 patients, 6.4%).

Five (10.6%) patients had TEAEs leading to definitive study treatment discontinuation. In addition to 4 patients with fatal events described above (acute myocardial infarction, sepsis, rectal hemorrhage and sepsis, sudden death), there was 1 patient with serious Grade 3 spinal cord compression which was considered not related to study treatment. There was also 1 patient that selectively discontinued pomalidomide (continuing treatment with isatuximab and dexamethasone) due to non-serious Grade 1 events of tremor, gait disturbance, and flushing.

During the treatment period, 71.7% of patients had Grade 3 or 4 neutrophil count decreased (37.0% and 34.8%, respectively), 67.4% of patients had Grade 3 or 4 white blood cell decreased (55.2% and 15.2%, respectively), and 65.2% of patients had Grade 3 or 4 lymphocyte count decreased (54.3% and 10.9%, respectively). Grade 3 anemia was reported in 21.7% of the patients, and Grade 4 anemia was reported in none of the patients during treatment.

### Conclusions

The results from the primary safety analyses discussed in Examples 1A and 1B have confirmed the safety and feasibility of the administration of isatuximab from a fixed infusion volume. This example summarizes the main findings from the efficacy analysis at the final-cutoff, 10 months after the date of the first dose of the last enrolled patient.

A total of 47 patients were enrolled, and 22 patients (46.8%) patients were still receiving study treatment at the cut-off date.

The median number of cycles administered were 9 (range: 1-19). The median duration of infusion decreased from 3.70 hours during the first infusion to 1.85 hours during the second infusion, and to 1.25 hours for ≥3 infusions when the infusion was administered at the fixed infusion rate of 200 mL/hour. Notwithstanding the increased infusion rate and shorter infusion duration from the second infusion onwards, no IRs were observed following the first infusion.

Efficacy was observed in these 47 patients who received isatuximab administered from a fixed infusion volume in combination with pomalidomide and dexamethasone, with an ORR of 53.2% (95% CI: 38.1% to 67.9%), and median PFS and OS had not yet been reached at a median duration of 9.9 months follow-up. The 12-month probability of PFS was 55.7%, and the 12-month probability of OS was 70.6%. Of the 7 patients with prior exposure to daratumumab, there was 1 PR and 2 MRs for an ORR of 14.3% and a CBR of 42.9%; the ORR in the 40 patients without prior exposure to daratumumab was 60.0%. The responses were durable, and the median duration of response had not yet been reached. The other ORR subgroup analyses, based on prior exposure to pomalidomide and other treatments, as well as type of measurable M-protein, did not show evidence of major differences in response rate compared to the all treated population.

The efficacy data with fixed volume infusion were consistent with the data from a parallel study comparing isatuximab in combination with pomalidomide and dexamethasone vs. pomalidomide and dexamethasone in patients with refractory or relapsed and refractory multiple myeloma. In the parallel study, isatuximab was administered infused at a rate based on amount of protein/hour (mg/hr). In the present study, the ORR in patients not exposed to daratumumab was 60.0%, compared to 60.4% in the parallel study. In the present study the 1-year PFS rate was 55.7%, compared to 47.6% in the parallel study. In the present study, the 1 year OS rate was 70.6%, compared to 72.% in the parallel study. In the present study the time to first response was 0.95 months, compared to 1.94 months in the parallel study. In the present study, the median duration of response had not been reached, compared to 13.27 months in the parallel study.

The safety findings were consistent with those reported in Examples 1A and 1B, with no Grade >3 IRs and no IRs after the second infusion, and no new safety signal noted with fixed volume administration. The safety data is also consistent with the infusion schedule used with IPd in the parallel study. These results confirm the safety, efficacy and feasibility of isatuximab administered by a fixed infusion volume method.

### Example 2

### Exposure-Response Analysis and Disease Modeling for Selection of Optimal Dosing Regimen of Isatuximab as Single Agent in Patients with Multiple Myeloma

Exposure-Response (E-R) analysis and disease modeling of tumor burden were performed to evaluate the relationship between isatuximab exposure and efficacy outcomes, and to support dosing regimen selection for isatuximab as a single agent in relapsed/refractory multiple myeloma (RRMM) patients.

Study Design

194 RRMM patients were administered isatuximab intravenously at doses ranging from 1 mg/kg to 20 mg/kg. Isatuximab was administered as a monotherapy at the selected dose either once per week or every 2 weeks. As shown in Table 18, the median age was 63 years, 94.3% of patients had ≥3 prior treatment lines, and the median percent bone marrow plasma cell was 27.6.

TABLE 18
Patient characteristics.

| | N = 194 patients |
|---|---|
| Age | |
| Median | 63 |
| Min; Max | 38; 85 |
| Number of prior treatment lines | |
| 1 | 1 (0.5) |
| 2 | 10 (5.2) |
| ≥3 | 183 (94.3) |
| % Bone marrow plasma cell | |
| Median | 27.6 |
| Min; Max | 0.0; 100 |

The pharmacokinetics, best overall response (ORR), and serum M-protein data (subset of 122 patients) were used for the ER analyses and disease modeling described in this Example.

Exposure-Response Analysis

Logistic regression modeling was used to examine the association of several isatuximab exposure parameters, including C_{trough} and percent bone marrow plasma cells, with the probability of achieving an objective response (CR, VGPR, or PR; see Table 14 for response criteria).

Baseline covariates were also considered in the model to reduce potential confounding effects. C_{trough} was defined as the plasma concentration of isatuximab observed just prior to treatment administration during repeated dosing.

Disease Progression Modeling

Disease progression was captured in a subset of 122 evaluable patients with the dynamics of serum M-protein. Drop outs were accounted using a joint model.

A Tumor Growth Inhibition (TGI) model (Claret et al., J Clin Oncol 27 (2009) 25:4103-4108; Jonsson et al., CPT Pharmacometrics Syst Pharmacol (2015) 4(12):711-719) was applied to longitudinal dynamics of the serum M-protein in 122 of the 194 RRMM patients administered isatuximab monotherapy intravenously at doses from 1 mg/kg to 20 mg/kg either once per week or every 2 weeks. Patient dropouts were accounted for using a joint model.

Trial Simulations

Clinical trial simulations (5000 trial simulations with 100 patients each) based on both the E-R analysis and TGI modeling described above were then carried out to evaluate different dosing regimens of interest using both models (E-R analysis and disease progression model).

Results

Logit Emax Model

Pharmacokinetics data was best described by a two-exponential distribution model with parallel linear and non-linear (target specific mediated) clearance.

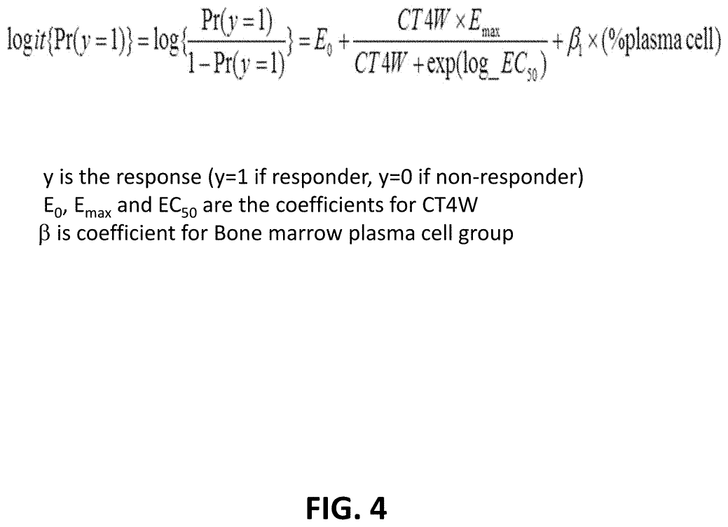

The relationship between isatuximab exposure and ORR was best described by a Logit Eₘₐₓ model (AUC of ROC curve=0.91) (FIG. 4). Table 19 provides parameter estimates of the Logit Emax model.

TABLE 19
Parameter estimates of the Logit Emax model.

| | | | | 95% Confidence |
|---|---|---|---|---|
| Parameter | Estimate | Standard Error | P-value | Limits |
| E₀ | −3.0063 | 0.9560 | 0.0019 | −4.8918-−1.1208 |
| Emax | 2.7205 | 0.9211 | 0.0035 | 0.9038-4.5372 |
| Log_EC₅₀ | 2.6584 | 1.0985 | 0.0164 | 0.4919-4.8248 |
| β | −1.0480 | 0.4885 | 0.0331 | −2.0114-−0.0847 |

The model revealed that C_{trough} at 4 weeks (CT4W) and the percent of bone marrow plasma cell (BMPC) were significant predictors of the overall response rate (ORR).

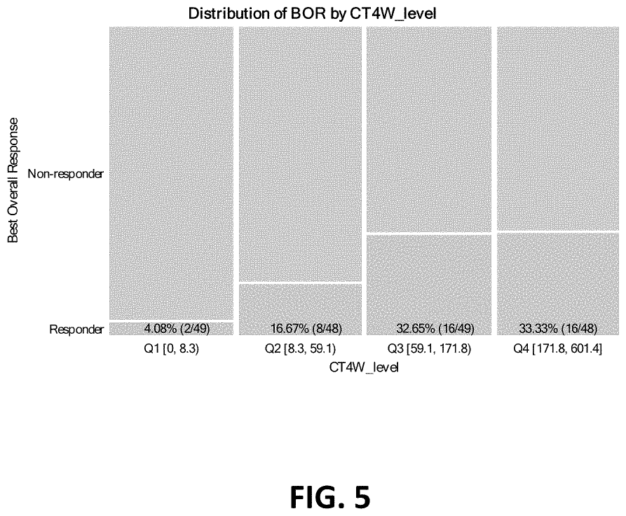

ORR increased as CT4W increased, with a plateau of ORR at about 33% reached for CT4W from the 3^{rd} quartile (FIG. 5). The CT4W value to provide 90% maximal effect (EC₉₀) was 128.5 μg/mL. Therefore, limited additional benefit in ORR is expected with CT4W higher than predicted EC₉₀.

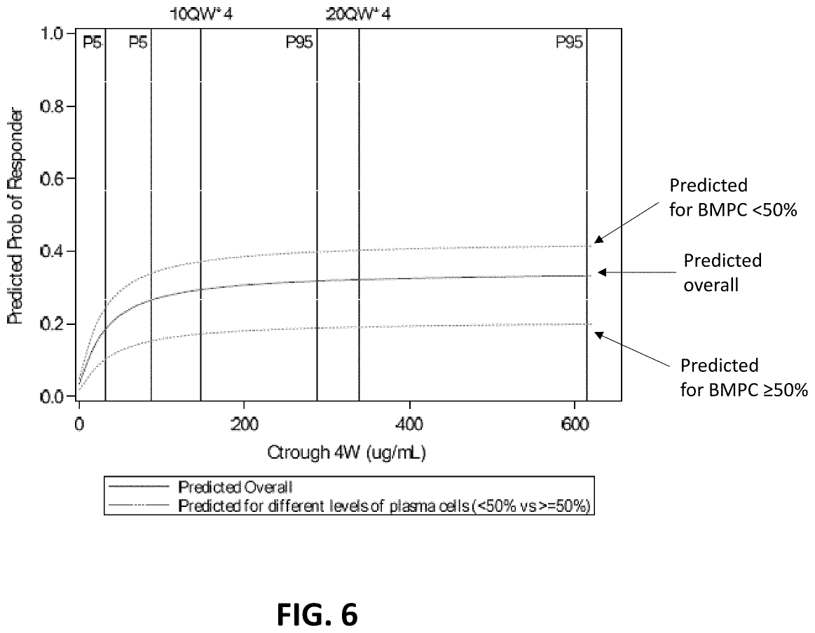

Patients with BMPC lower than 50% were more likely to respond (FIG. 6). For a given BMPC value, higher probability of response to treatment was obtained with higher CT4W.

M-Protein Model

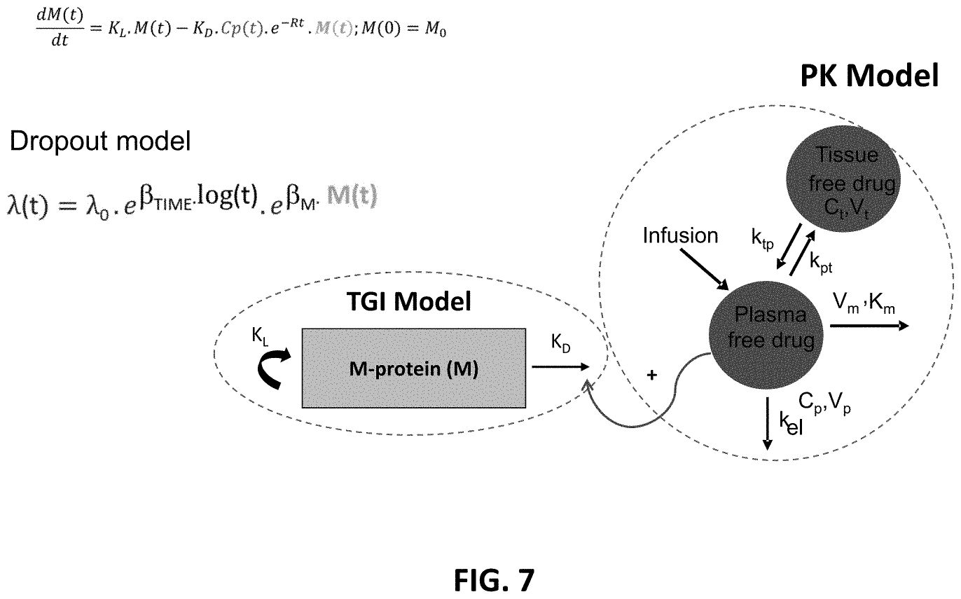

Serum M-protein kinetics were adequately described by an exposure-driven TGI model (FIG. 7) (Claret et al., J Clin Oncol 27 (2009) 25:4103-4108; Jonsson et al., CPT Pharmacometrics Syst Pharmacol (2015) 4912):711-719). The parameter estimates of disease of the M-protein model at provided in Table 20.

TABLE 20
Parameter estimates of disease M-protein model.

| | | Interindividual | Residual variability | |
|---|---|---|---|---|
| Fixed effects | | variability | (combined error) | |
| | Estimate | ω (%) | σa (g/L) | σp (%) |
| Parameter | (RSE %) | (RSE %) | (RSE %) | (RSE %) |
| M0 (g · L⁻¹) | 24.4 (6) | 65.4 (7) | 0.927 (14) | 6.07 (15) |
| KL (day⁻¹) | 0.00407 (15) | 105 (13) | | |
| KD (L · mg⁻¹ · | 0.000121 (27) | 154 (12) | | |
| day⁻¹) | | | | |
| R (day⁻¹) | 0.0198 (28) | 123 (15) | | |
| λ₀ | 2.73e−05 (41) | — | | |
| β_{TIME} | 0.85 (8) | — | | |
| βM | 0.0476 (8) | — | | |

K_{L}: tumor growth rate; K_{D}: drug constant-cell-kill rate; R: resistance appearance rate; M₀: M-protein at baseline; λ₀ baseline hazard; b_{M}: link between M-protein and risk of dropout.

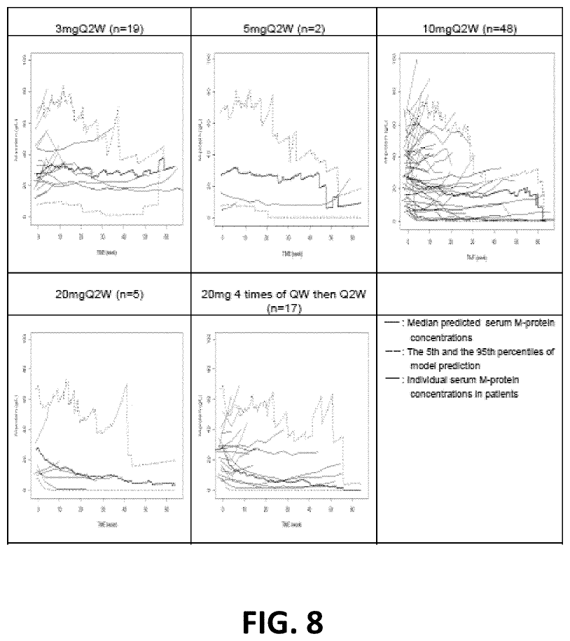

As shown in FIG. 8, the disease M-protein model adequately described the observed time-course of serum M-protein levels.

Clinical Trial Simulations

5000 clinical trial simulations with 100 patients each were carried out. The models assumed that patients receive the same dose level for each simulated trial.

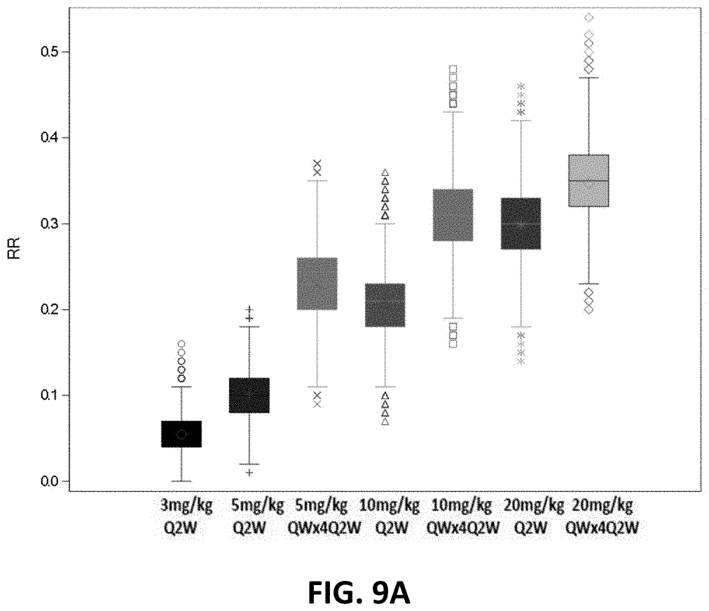

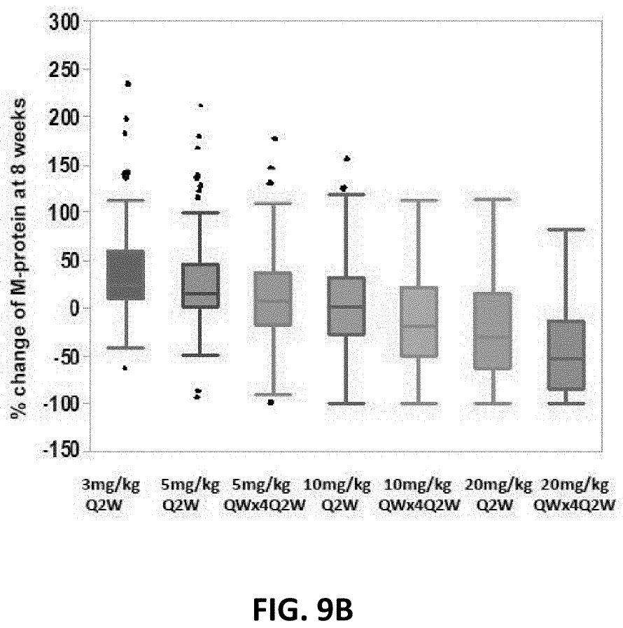

As shown in FIGS. 9A and 9B, the clinical trial simulations revealed that weekly administration together with a high dose at the first cycle (loading dose period) allow optimization of the response as the efficacious concentration is more rapidly reached.

The probabilities of success to reach a 30% ORR with several dosing regimens are provided in Table 21. FIG. 9A provides simulated overall response rates at several dosing regimens, including the 20 mg/kg QWX4Q2W dosing regimen.

TABLE 21
Simulated probabilities of reaching 30% ORR with the indicated dosing regimens.

| | 3 mg/kg | 5 mg/kg | 5 mg/kg | 10 mg/kg | 10 mg/kg | 20 mg/kg | 20 mg/kg |
|---|---|---|---|---|---|---|---|
| | Q2 W | Q2 W | QW × 4Q2 W | Q2 W | QW × 4Q2 W | Q2 W | QW × 4Q2 W |
| Prob | 0 | 0.32 | 17.9 | 12.58 | 45.64 | 41.22 | 59.72 |
| (ORR ≥30) | | | | | | | |

The median percent change of M-protein levels at two months relative to baseline are provided in Table 22 for several dosing regimens. The QWX4Q2W led to a 52% reduction of serum M-protein from baseline levels following 2 months of treatment (FIG. 9B).

TABLE 22
Simulated percent change of M-protein at two months from baseline.

| | 3 mg/kg | 5 mg/kg | 5 mg/kg | 10 mg/kg | 10 mg/kg | 20 mg/kg | 20 mg/kg |
|---|---|---|---|---|---|---|---|
| | Q2 W | Q2 W | QW × 4Q2 W | Q2 W | QW × 4Q2 W | Q2 W | QW × 4Q2 W |
| Median | 24.1 | 14.9 | 7.2 | 1.3 | −17.9 | −30.5 | −52.1 |
| 5-95^{th} | [−8.9, | [−33.4, | [−72.8, | [−84.1, | [−96.7, | [−99, | [−100, |
| percentiles | 139.8] | 134.9] | 129.5] | 123.5] | 93.1] | 74.7] | 36.4] |

In addition, the QWX4Q2W isatuximab dosing regimen appeared to be well tolerated.

Conclusions

The present Example demonstrates that a model-based drug development approach has been successfully applied to support phase II isatuximab monotherapy dosing regimen selection in RRMM patients. This approach showed that a loading dose of isatuximab 20 mg/kg weekly over only 4 weekly administrations, followed by administration every 2 weeks appeared adequate to maximize the tumor response and sustain efficacy in monotherapy while being well tolerated.

The dose recommendation of 20 mg/kg QW/Q2W applies to monotherapy.

### Example 3

### A Phase 1/2 Study of Isatuximab Monotherapy for Relapsed and/or Refractory Multiple Myeloma in Japanese Patients

This example describes a Phase 1/2 study of isatuximab monotherapy for relapsed and/or refractory multiple myeloma (RRMM) in Japanese patients.

### Study Objectives

Phase 1: To evaluate safety and tolerability, and dose-limiting toxicities (DLTs) of isatuximab in Japanese patients with RRMM.

Phase 2: To evaluate the efficacy of isatuximab at the recommended dose, and to determine its overall response rate (ORR; ≥partial response [PR]) in Japanese patients with RRMM.

### Study Population

Patients meeting the following criteria were enrolled in this study:
Patients aged ≥20 years with a diagnosis of symptomatic multiple myeloma, at least three prior lines of therapy or refractory to both an IMiD® and a proteasome inhibitor (PI), with a minimal response or better to at least one line, refractory to most recent therapy, and measurable disease.
RRMM was diagnosed according to International Myeloma Working Group criteria (Palumbo A et al. J Clin Oncol 2014; 32: 587-600) and staged according to International Staging System (Greipp P R et al., J Clin Oncol 2005; 23: 3112-20).

Key exclusion criteria were: prior treatment with an anti-CD38 agent; diagnosis of another malignancy within 5 years of enrollment; prior anticancer therapy within 21 days of first drug infusion; systemic radiotherapy within 4 weeks or localized radiotherapy within 1 week prior to first drug infusion; abnormal laboratory values; ongoing toxicity of grade≥2; prior allogenic stem cell transplantation; or diagnosis of Crow-Fukase syndrome, plasma cell leukemia, Waldenstrom's macroglobulinemia, or multiple myeloma of the IgM subtype.

### Study Design

This study is an open-label, non-randomized, single-arm, two-phase, multicenter trial performed in Japan. The trial comprised a dose-escalation phase (Phase 1) to determine the maximum tolerated dose based on dose-limiting toxicities (DLTs), followed by a confirmatory phase (Phase 2), which enrolled patients at the maximum tolerated dose determined in Phase 1.

Phase 1

The maximum tolerated dose (MTD) of isatuximab monotherapy was determined in two cohorts of patients in a 3+3 design:
Cohort 1: Isatuximab administered in 28-day cycles at 10 mg/kg every week (QW) in cycle 1 (i.e., four weeks) and every 2 weeks (Q2W) in subsequent four-week cycles.
Cohort 2: Isatuximab administered in 28-day cycles at 20 mg/kg QW in cycle 1 and Q2W in subsequent cycles; enrollment started after completion of the DLT observation period in Cohort 1.

The dose regimens used in Phase 1 were selected as half the highest dose (Cohort 1) and the highest dose (Cohort 2) used in study Martin T G et al., J Clin Oncol 2014; 32:abstract 8532.

Phase 2

Patients received the MTD established in Phase 1. Enrollment commenced after completion of the DLT observation period in Cohort 2. Patients in Phase 2 included those patients enrolled in the Phase 1 cohort treated with the recommended dose.

Study Endpoints

Primary Endpoints

The primary endpoints of this study were the safety and tolerability of isatuximab in Phase 1, including DLTs, and to evaluate the efficacy of isatuximab at the recommended dose, including assessment of ORR.

Secondary Endpoints

The secondary endpoints included:
Safety and immunogenicity (antidrug antibodies [ADA]) of isatuximab.
Pharmacokinetics of isatuximab.
Efficacy determined using IMWG uniform response criteria, ORR, clinical benefit rate (CBR), overall survival (OS), and progression-free survival (PFS).
Best response in paraprotein.
Baseline CD38 receptor density (RD) on multiple myeloma cells.

Exploratory Objectives

The exploratory objectives included:
Minimal residual disease (MRD), assessed in patients achieving complete response (CR), and its correlation with clinical outcomes.

Statistical Analyses

ORR was assessed in all patients who received at least one dose of isatuximab at the recommended dose in either Phase 1 or Phase 2. The null hypothesis that the true response rate was <10% was tested using a one-sided exact binomial test with a significance level of 0.025 assuming true ORR of 28%.

### Results

Patient Demographics and Baseline Characteristics

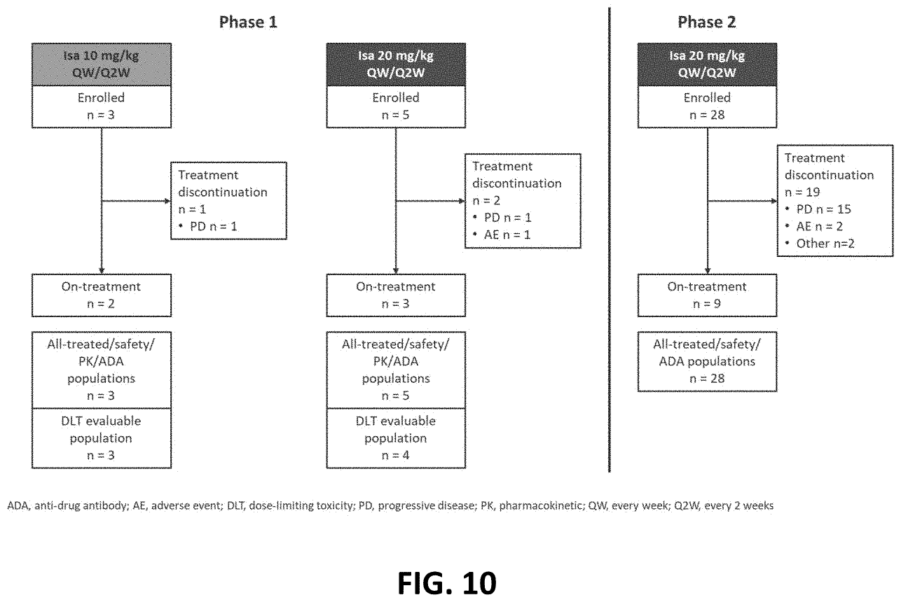

As shown in FIG. 10, eight patients were enrolled in Phase 1 and 28 patients were enrolled in Phase 2. All patients had received at least two prior therapies, including an IMiD® and a PI, and the majority was refractory to an IMiD® and/or a PI (Table 23).

TABLE 23
Patient characteristics.

| | Phase 1 | | Phase 2 | All Treated |
|---|---|---|---|---|
| | Isa 10 | Isa 20 | Isa 20 | Isa 20 |
| | mg/kg | mg/kg | mg/kg | mg/kg |
| | QW/Q2W | QW/Q2W | QW/Q2W | QW/Q2W |
| | (n = 3) | (n = 5) | (n = 28) | (n = 33) |
| Male/female, n | 1/2 | 1/4 | 18/10 | 19/14 |
| Age in years, median (range) | 69.0 (59-74) | 76.0 (69-80) | 71.5 (48-82) | 72.0 (48-82) |
| Weight in kg, median (range) | 44.40 (43.6- | 48.70 (37.6- | 56.30 (38.8- | 55.3 (37.6- |
| | 73.4) | 66.0) | 75.0) | 75.0) |
| ECOG PS, 0/1/2, n | 2/1/0 | 2/2/1 | 15/9/4 | 17/11/5 |
| Time from diagnosis to first | 6.69 (4.8- | 4.25 (1.6- | 6.24 (1.4- | 5.46 (1.4- |
| dose of isatuximab in years, | 18.0) | 6.6) | 18.6) | 18.6) |
| median (range) | | | | |
| ISS at initial diagnosis, | 0/2/0/1 | 1/2/2/0 | 10/11/4/3 | 11/13/6/3 |
| I/II/III/unknown | | | | |
| Measurable paraprotein, | 3/0/0 | 3/1/1 | 21/3/4 | 24/4/5 |
| serum/urine/both, n | | | | |
| Multiple Myeloma subtype, n | | | | |
| (%) | | | | |
| Heavy chain | | | | |
| IgA | 0 | 0 | 6 (21) | 6 (18) |
| IgD | 0 | 0 | 1 (4) | 1 (3) |
| IgG | 3 (100) | 4 (80) | 19 (68) | 23 (70) |
| Not applicable | 0 | 0 | 1 (4) | 1 (3) |
| Undetected | 0 | 1 (20) | 1 (4) | 2 (6) |
| Light chain | | | | |
| Kappa | 2 (67) | 3 (60) | 17 (61) | 20 (61) |
| Lambda | 1 (33) | 2 (40) | 11 (39) | 13 (39) |
| Biclonal (no) | 3 (100) | 5 (100) | 28 (100) | 33 (100) |
| Median plasma cells in | 6.20 (0.0- | 15.80 (6.6- | 14.50 (0.4- | 15.60 (0.4- |
| marrow (range), % | 45.8) | 81.8) | 84.6) | 84.6) |
| Patients with plasmacytomas, | 1 (33) | 1 (20) | 5 (18) | 6 (18) |
| n (%) | | | | |
| Patients with bone lesions, n | 2 (67) | 5 (100) | 15 (54) | 20 (61) |
| Derived ISS at study entry, n | | | | |
| (%) | | | | |
| I | 1 (33) | 1 (20) | 14 (50) | 15 (45) |
| II | 1 (33) | 2 (40) | 9 (32) | 11 (33) |
| III | 1 (33) | 2 (40) | 5 (18) | 7 (21) |
| Median serum β2-MG | 5.10 (2.8- | 4.50 (2.5- | 3.30 (1.9- | 3.40 (1.9- |
| (range), mg/L | 5.8) | 10.4) | 12.7) | 12.7) |
| Median albumin (range), g/L | 35.00 | 38.00 | 36.50 (18.0- | 37.00 |
| | (34.0-37.0) | (23.0-40.0) | 42.0) | (18.0-42.0) |
| High risk cytogenetic | | | | |
| abnormalities at study entry | | | | |
| At least one cytogenetic | 2 (67) | 1 (20) | 8 (29) | 9 (27) |
| abnormality | | | | |
| At least two cytogenetic | 1 (33) | 1 (20) | 3 (11) | 4 (12) |
| abnormalities | | | | |
| 17p deletion (TP53) | 2 (67) | 1 (20) | 5 (18) | 6 (18) |
| t(4; 14) translocation | 1 (33) | 1 (20) | 5 (18) | 6 (18) |
| (FGFR3/IGH) | | | | |
| t(14; 16) translocation | 0 | 0 | 1 (4) | 1 (3) |
| (IGH/MAF) | | | | |
| Number of prior treatment | 5.0 (4-12) | 4.0 (3-6) | 5 (2-11) | 5.0 (2-11) |
| lines, median (range) | | | | |
| Prior therapies, n (%) | | | | |
| ImiD ® | 3 (100) | 5 (100) | 28 (100) | 33 (100) |
| Lenalidomide | 3 (100) | 5 (100) | 27 (96) | 32 (97) |
| Pomalidomide | 3 (100) | 3 (60) | 22 (79) | 25 (76) |
| Thalidomide | 1 (33) | 0 | 8 (29) | 8 (24) |
| PI | 3 (100) | 5 (100) | 28 (100) | 33 (100) |
| Bortezomib | 3 (100) | 5 (100) | 27 (96) | 32 (97) |
| Carfilzomib | 1 (33) | 2 (40) | 9 (32) | 11 (33) |
| Ixazomib | 0 | 1 (20) | 3 (11) | 4 (12) |
| Other | 1 (33) | 2 (40) | 10 (36) | 12 (36) |
| Panobinostat | 1 (33) | 1 (20) | 7 (25) | 8 (24) |
| Elotuzumab | 0 | 1 (20) | 6 (21) | 7 (21) |
| IMiD ® plus PI | 3 (100) | 5 (100) | 28 (100) | 33 (100) |
| Lenalidomide and | 3 (100) | 5 (100) | 26 (93) | 31 (94) |
| bortezomib | | | | |
| Lenalidomide, | 1 (33) | 1 (20) | 6 (21) | 7 (21) |
| bortezomib, | | | | |
| pomalidomide and | | | | |
| carfilzomib | | | | |
| Refractory to IMiD ®, n | 3 (100) | 5 (100) | 25 (89) | 30 (91) |
| Refractory to PI, n | 2 (67) | 4 (80) | 25 (89) | 29 (88) |
| Refractory to IMiD ® and PI, | 2 (67) | 4 (80) | 22 (79) | 26 (79) |
| n | | | | |

QW, every week; Q2W, every 2 weeks; ECOG, Eastern Cooperative Oncology Group; PS, Performance Status; IMiD ®, immunomodulatory drug; PI, protease inhibitor; β2-MG, β2-microglobulin.

The number of cycles ranged from 1 to 24, the duration of exposure ranged from 2 to 96 weeks, and the cumulative dose ranged from 40.0 to 859.3 mg/kg (Table 24).

TABLE 24
Isatuximab exposure.

| | | Phase 1 | | Phase 2 |
|---|---|---|---|---|
| | | Isa 10 mg/kg | Isa 20 mg/kg | Isa 20 mg/kg |
| | | QW/Q2W | QW/Q2W | QW/Q2W |
| | | (n = 3) | (n = 5) | (n = 28) |
| Number of cycles, median (range) | | 22.0 (2-24) | 15.0 (1-21) | 6.0 (1-13) |
| Duration of exposure in weeks, median | | 90.4 (6-96) | 57.9 (2-82) | 22.0 (4-50) |
| (range) | | | | |
| Cumulative dose in mg/kg, median | | 449.10 (50.0- | 619.30 (40.0- | 259.60 (77.8- |
| (range) | | 490.0) | 859.3) | 520.3) |
| ADI in | Cycle 1 | 10.00 (7.4- | 19.95 (15.3- | 20.00 (13.4- |
| mg/kg/week, | | 10.0) | 20.7) | 21.3) |
| median (range) | | | | |
| | Subsequent cycles | 4.89 (4.8-5.0) | 9.93 (9.8-10.0) | 10.00 (6.8- |
| | | | | 10.9) |
| RDI in %, | Cycle 1 | 100.00 (73.7- | 99.75 (76.3- | 100.00 (66.8- |
| median (range) | | 100.0) | 103.6) | 106.4) |
| | Subsequent cycles | 97.83 (96.3- | 99.27 (98.0- | 100.00 (68.0- |
| | | 100.0) | 99.9) | 108.5) |

QW, every week; Q2W, every 2 weeks; ADI, actual dose intensity; RDI, relative dose intensity.

Five patients in Phase 1 and nine patients in Phase 2 were still on treatment at the cutoff date.

Safety

Dose-Limiting Toxicities (DLTs)

One patient was excluded from the DLT evaluable population owing to adverse events (both AEs were unrelated to isatuximab).

No DLTs occurred in either cohort in Phase 1. Accordingly, the starting dose in Phase 2 was set as 20 mg/kg QW/Q2W.

Adverse Events

Treatment-emergent AEs (TEAEs) in both phases are summarized in Table 25 by dose and grade. The only serious drug-related TEAE was grade≥3 pneumonia, which occurred in 1 patient treated with 10 mg/kg QW/Q2W in Phase 1 and in two patients in Phase 2.

Infusion-related reactions occurred in 3 Phase 1 patients (2 events in 2 patients at 10 mg/kg, and 2 events in 1 patient at 20 mg/kg), and in 12 patients in Phase 2 (13 events). All infusion-related reactions were grade≤2. When a reaction occurred, it was at the first infusion in all patients in Phase 1 and in 11 patients in Phase 2. One patient in Phase 2 experienced reactions at the first and third infusion. All infusion-related reactions resolved within 1 day, except two patients with reactions that lasted 2 days. No patients discontinued treatment due to infusion reactions.

Clinically significant TEAEs that occurred in all 36 patients in Phases 1 and 2, combined, were respiratory infections in 19 patients, lower respiratory TEAEs in 8 patients, and neutropenia in 13 patients.

TABLE 25
Summary of safety results.

| | Phase 1 | | | | Phase 2 | |
|---|---|---|---|---|---|---|
| | Isa 10 mg/kg | | Isa 20 mg/kg | | Isa 20 mg/kg | |
| | QW/Q2 W (n = 3), | | QW/Q2 W (n = 5), | | QW/Q2 W (n = 28), | |
| | n (%) | | n (%) | | n (%) | |
| | Any grade | Grade ≥3 | Any grade | Grade ≥3 | Any grade | Grade ≥3 |
| Any TEAE | 3 (100) | 1 (33.3) | 4 (80.0) | 2 (40.0) | 25 (89.3) | 12 (42.9) |
| Drug-related TEAE | 2 (66.7) | 1 (33.3) | 1 (20.0) | 0 | 18 (64.3) | 3 (10.7) |
| Serious TEAE | 1 (33.3) | | 1 (20.0) | | 7 (25.0) | |
| Serious drug-related TEAE | 1 (33.3) | | 0 | | 2 (7.1) | |
| TEAE leading to death | 0 | | 1 (20.0) | | 0 | |
| TEAE leading to | 0 | | 1 (20.0) | | 2 (7.1) | |
| discontinuation | | | | | | |
| At least one DLT | 0 | | 0 | | — | |
| At least one infusion-related | 2 (66.7) | 0 | 1 (20.0) | 0 | 12 (42.9) | 0 |
| reaction | | | | | | |
| Frequent TEAEs (in ≥3 | | | | | | |
| patients in either Phase) | | | | | | |
| Nasopharyngitis | 2 (66.7) | 0 | 1 (20.0) | 0 | 6 (21.4) | 0 |
| Vomiting | 1 (33.3) | 0 | 2 (40.0) | 0 | 1 (3.6) | 0 |
| Pneumonia | 1 (33.3) | 1 (33.3) | 1 (20.0) | 1 (20.0) | 3 (10.7) | 2 (7.1) |
| Rhinorrhea | 1 (33.3) | 0 | 1 (20.0) | 0 | 3 (10.7) | 0 |
| Cataract | 1 (33.3) | 0 | 0 | 0 | 3 (10.7) | 1 (3.6) |
| Diarrhea | 1 (33.3) | 0 | 0 | 0 | 3 (10.7) | 0 |
| Back pain | 1 (33.3) | 0 | 0 | 0 | 4 (14.3) | 0 |
| Pyrexia | 0 | 0 | 0 | 0 | 6 (21.4) | 1 (3.6) |
| Edema peripheral | 0 | 0 | 0 | 0 | 3 (10.7) | 1 (3.6) |

Immunogenicity

Anti-drug antibodies (ADA) were measured in all 36 patients. At the cutoff date, all patients in Phase 1 were negative for ADAs. In Phase 2, four patients showed evidence of treatment-induced immunogenicity, with transient ADA in 1 patient (Cycle 1 only) and treatment-boosted ADA in 3 patients. There was no relationship between trough isatuximab concentrations and immunogenicity.

Pharmacokinetics

The duration of infusion in Cycle 1 was longer in patients who received isatuximab at 20 mg/kg QW/Q2W (Table 26; infusion rate was measured as mg/hr).

TABLE 26
Pharmacokinetic parameters of isatuximab in Phase 1, Cycle 1.

| | | Phase 1 | |
|---|---|---|---|
| | | Isa 10 mg/ | Isa 20 mg/ |
| | | kg QW/Q2W | kg QW/Q2W |
| | | (n = 3) | (n = 5) |
| Patients with available PK | | 3 | 4 |
| data, n | | | |
| Infusion duration (h), median | | 2.63 (2.32-3.23) | 3.82 (3.28-6.05) |
| (range) | | | (n = 5) |
| Cₑₒᵢ | Mean (SD) | 122 (21.6) | 246 (52) |
| (μg/mL) | Geometric mean | 121 (18) | 242 (21) |
| | (CV %) | | |
| Cₘₐₓ | Mean (SD) | 124 (22.9) | 280 (64.4) |
| (μg/mL) | Geometric mean | 123 (18) | 274 (23) |
| | (CV %) | | |
| AUC_{1 week} | Mean (SD) | 9,300 (3,010) | 21,300 (5,520) |
| | Geometric mean | 8,970 (32) | 20,800 (26) |
| | (CV %) | | |

| | | |
|---|---|---|
| Tₘₐₓ (h), median (range) | 2.68 (2.32-7.25) | 5.56 (3.28-8.48) |

QW, every week; Q2W, every 2 weeks; Cₑₒᵢ, concentration at the end of infusion; Cₘₐₓ, maximum concentration; AUC_{1 week}, area under the plasma concentration versus time in the 1-week dosing interval; tₘₐₓ, time to reach Cₘₐₓ.

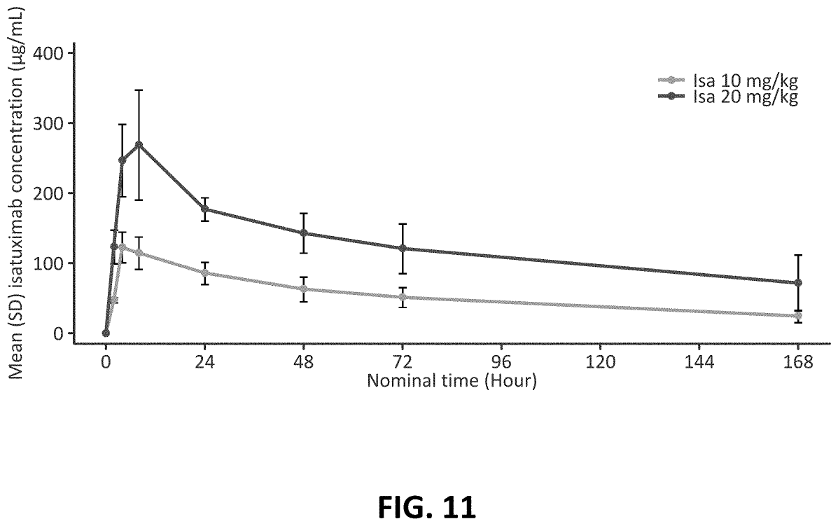

As shown in FIG. 11, the 2-fold increase in dose (from 10 mg/kg to 20 mg/kg) increased isatuximab exposure by 2.3-fold.

Efficacy

As shown in Table 27A, the ORR was assessed in 33 patients who received isatuximab at 20 mg/kg QW/Q2W in Phase 1 Cohort 2 or in Phase 2. The ORR (≥PR) was 36.4% (95% CI: 20.4%, 54.9%; 12/33 patients), which significantly exceeded the null hypothesis rate of <10% based on a one-sided exact binominal test with a significance level of 0.025 (P<0.0001). The CBR (≥MBR) was 54.5% (95% CI: 36.4%, 71.9%; 18/33 patients). Among all enrolled patients, CR was achieved in 2 patients, VGPR in 5 patients, and PR in 5 patients. There appeared to be no differences in the response rate according to the number of prior lines or cytogenetic risk. Among eight patients with cytogenetic abnormalities, the response was ≥PR in three patients and VGPR in two patients. All three patients with ≥PR had the t(4,14) cytogenetic abnormality. In other subgroups of patients, Response rates tended to be greater in patients with low ECOG, low ISS grade, baseline creatinine clearance≥60 mL/min/1·73 m², and absence of plasmacytoma at screening

TABLE 27A
Best overall responses.

| | Phase 1 | | Phase 2 | All Treated |
|---|---|---|---|---|
| | Isa 10 mg/kg | Isa 20 mg/kg | Isa 20 mg/kg | Isa 20 mg/kg |
| | QW/Q2W | QW/Q2W | QW/Q2W | QW/Q2W |
| | (n = 3), | (n = 5), | (n = 28), | (n = 33), |
| | n (%) | n (%) | n (%) | n (%) |
| ORR (≥PR) | 2 (66.7) | 3 (60.0) | 9 (32.1) | 12 (36.4)** |
| | | | | (95% CI: 20.4- |
| | | | | 54.9) |
| CBR (≥MR) | 2 (66.7) | 3 (60.0) | 15 (53.6) | 18 (54.5) |
| | | | | (95% CI: 36.4- |
| | | | | 71.9) |
| Best response | | | | |
| CR | 0 | 1 (20.0) | 1 (3.6) | 2 (6.1) |
| VGPR | 1 (33.3) | 1 (20.0) | 3 (10.7) | 4 (12.1) |
| PR | 1 (33.3) | 1 (20.0) | 5 (17.9) | 6 (18.2) |
| MR | 0 | 0 | 6 (21.4) | 6 (18.2) |
| SD | 0 | 0 | 7 (25.0) | 7 (21.2) |
| PD | 0 | 1 (20.0) | 3 (10.7) | 4 (12.1) |
| Unconfirmed | 1 (33.3) | 0 | 2 (7.1) | 2 (6.1) |
| PD | | | | |
| NE | 0 | 1 (20.0) | 1 (3.6) | 2 (6.1) |

QW, every week; Q2W, every 2 weeks; ORR, overall response rate; PR, partial response; CBR, clinical benefit rate; MR, minimal response; CR, complete response; VGPR, very good partial response; SD, stable disease; PD, progressive disease; NE, not evaluable.
**P < 0.0001.

Patients were followed up for 4.1 to 90.1 weeks from the start of isatuximab therapy (Table 27B), with a median follow-up of 84.6 and 52.0 weeks in the 10 and 20 mg/kg QW/Q2W groups in Phase 1 and 19.2 weeks in Phase 2. The median duration of response in the three groups was 82.6, 48.1, and 241 weeks, respectively.

TABLE 27B
Secondary efficacy outcomes by study phase and dose

| | Phase 1 | | Phase 2 |
|---|---|---|---|
| | 10 mg/kg | 20 mg/kg | 20 mg/kg |
| | QW/Q2W | QW/Q2W | QW/Q2W |
| | (n = 3) | (n = 5) | (n = 28) |
| Median follow-up | 84.6 (4.1-90.1) | 52.0 (5.0-76.1) | 19.2 (3.6-44.6) |
| (range), weeks | | | |
| Median duration of | 82.6 | 48.1 | 24.1 |
| response (range), weeks | (79.1-86.1) | (48.1-50.3) | (11.6-36.3) |
| Median time to first | 4.9 | 5.4 (4.0-28.1) | 43 (4.1-12.1) |
| response (range), weeks | (4.1-5.6) | | |

QW, every week; Q2W, every 2 weeks

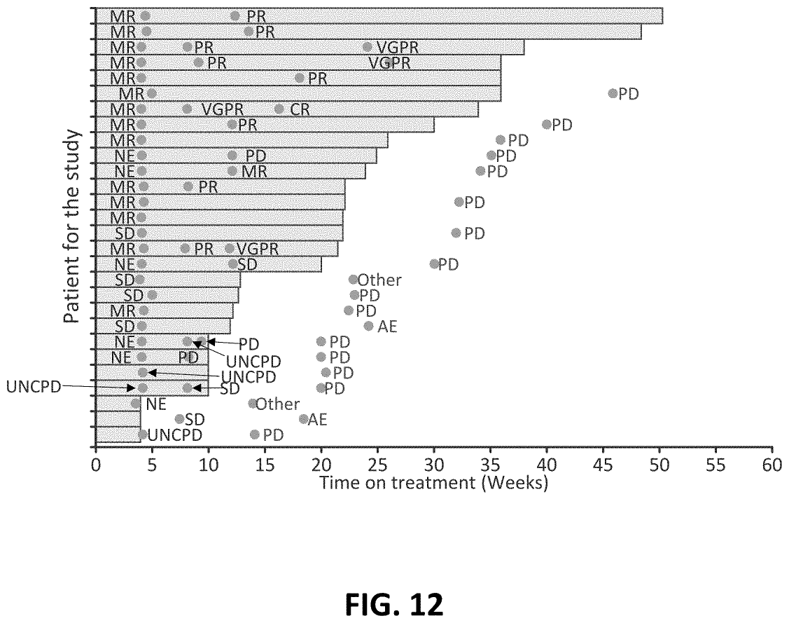

FIG. 12 shows the best response as a function of time on treatment in Phase 2. The median time to first response was comparable in all three groups (4.9, 5.4, and 4.3 weeks, respectively).

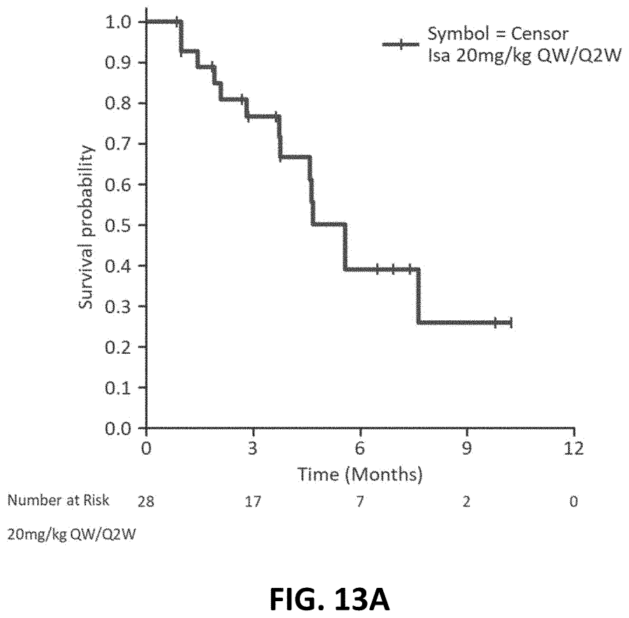

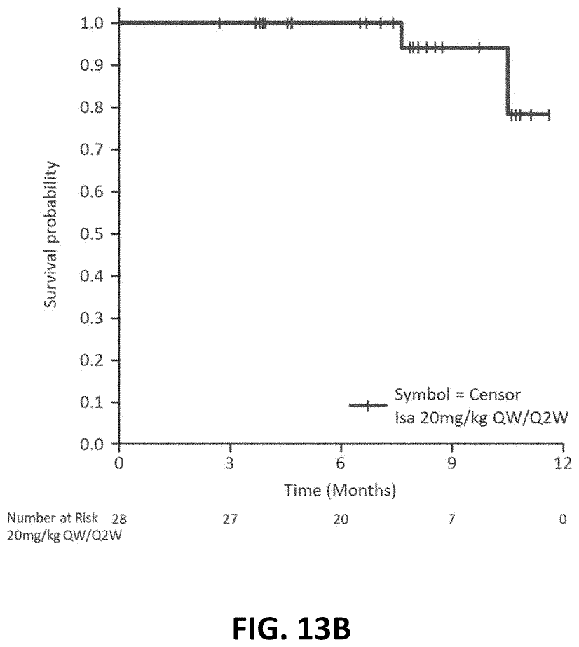

FIG. 13A provides a Kaplan-Meier plot of progression-free survival for the 28 patients in Phase 2 of this study. As shown in Table 28, the median PFS was about 4.7 months (95% CI: 3.75 to not reached).

TABLE 28
Kaplan-Meier statistics for progression-free survival.

| Outcome | Value |
|---|---|
| Patients with an event, n (%) | 14 (50.0) |
| Patients censored, n (%) | 14 (50.0) |
| Kaplan-Meier estimate (95% CI) | |
| 25^{th} percentile | 3.7 (1.87 to 4.67) |
| Median | 4.7 (3.75 to NC) |
| 75^{th} percentile | NC (4.55 to NC) |

CI, confidence interval; NC, not calculable; QW, every week; Q2W, every 2 weeks.

FIG. 13B provides a Kaplan-Meier plot of overall survival for patients in Phase 2 of this study. As shown in Table 29, the median OS was not reached. The OS probabilities at 6 months and 1 year were 1.000 and 0.781, respectively. There were two deaths in Phase 2. Both patients died during the post-treatment period and the causes of death were not related to AEs with study treatment. One of these patients did not receive subsequent therapy and the other was treated with carfilzomib and dexamethasone after isatuximab discontinuation.

TABLE 29
Kaplan-Meier statistics for overall survival.

| Outcome | Value |
|---|---|
| Patients with an event, n (%) | 2 (7.1) |
| Patients censored, n (%) | 26 (92.9) |
| Kaplan-Meier estimate (95% CI) | |
| 25^{th} percentile | NC (10.51 to NC) |
| Median | NC (10.51 to NC) |
| 75^{th} percentile | NC (NC to NC) |

CI, confidence interval; NC, not calculable; QW, every week; Q2W, every 2 weeks.

Best Response in Paraprotein

About half of all patients had a ≥50% reduction in paraprotein, with a reduction of ≥90% in 4 patients in Phase 1 (one at 10 mg/kg QW/Q2W and three at 20 mg/kg QW/Q2W) and 6 patients in Phase 2. There was no clear correlation between the best percent change in paraprotein and overall response.

Minimal Residual Disease (MRD)

MRD was assessed in three patients. Of two patients achieving CR, one patient in the 20 mg/kg group in Phase 1 was MRD negative and one patient in Phase 2 was MRD positive (at the 10⁻⁵ threshold). The patient with VGPR in the 10 mg/kg group in Phase 1 was MRD positive at 10⁻⁵.

Biomarkers

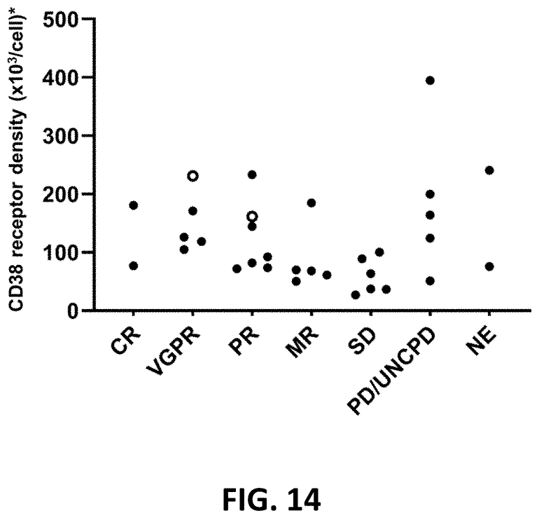

CD38 RD data were available for 32 patients. CD38 receptor density (×10³/cell) was calculated the specific molecule equivalent per cell (sMEC), using the conversion formula: sMEC=MEC(selected antibody)−MEC(negative isotypic control), where MEC (molecule equivalent per cell)=10\hat{ }(log[MFI]×a+b), in which a and b are the slope and the y-intercept of the calibration curve equation, respectively. CD38 RD was slightly higher in responders than in non-responders, with median (range) values of 122,313·5 (71,808 to 232,958) among 14 responders and 72,731·0 (26,921 to 394,910) among 18 non-responders. See FIG. 14 (CR=complete response; VGPR=very good partial response; PR=partial response; MR=minimal response; SD=stable disease; PD/UNCPD=progressive disease/unconfirmed progressive disease; NE=not evaluable). When patients were divided according to CD38 RD thresholds, the ORR tended to be greater in patients whose RD was above the threshold value. However, some patients with lower RD values showed responses to isatuximab.

Pharmacokinetics of Isatuximab

Pharmacokinetic properties of isatuximab on Cycle 1, in Phase 1, are shown in Table 30. The total variability of exposure parameters was low to moderate, with coefficients of variability of 18 to 32%. For a two-fold dose increase (from 10 to 20 mg/kg), isatuximab exposure increased by 2.3-fold (based on the geometric mean ratio).

TABLE 30
Isatuximab plasma pharmacokinetic parameters at Cycle 1 of Phase 1

| | Phase 1 | |
|---|---|---|
| | 10 mg/kg | 20 mg/kg |
| | QW/Q2W | QW/Q2W |
| | (n = 3) | (n = 5) |
| No of patients with evaluable | 3 | 4 |
| PK | | |
| Infusion duration, h | | |
| Median (range) | 2.63 (2.32-3.23) | 3.82 (3.28-6.05)^{a} |
| Cₑₒᵢ, μg/mL | | |
| Mean (SD) | 122 (21.6) | 246 (51.8) |
| Geometric mean (CV %) | 121 (18) | 242 (21) |
| Cₘₐₓ, μg/mL | | |
| Mean (SD) | 124 (22.9) | 280 (64.4) |
| Geometric mean (CV %) | 123 (18) | 274 (23) |
| AUC_{1 week} | | |
| Mean (SD) | 9,300 (3010) | 21,300 (5520) |
| Geometric mean (CV %) | 8,970 (32) | 20,800 (26) |
| tₘₐₓ, h | | |
| Median (range) | 2.68 (2.32-7.25) | 5.56 (3.28-8.48) |

^{a}n = 5
QW, every week; Q2W, every 2 weeks; Cₑₒᵢ, concentration at the end of infusion; Cₘₐₓ, maximum concentration; AUC_{1 week}, area under the plasma concentration versus time in the 1-week dosing interval; tₘₐₓ, time to reach Cₘₐₓ

### Conclusions

This study confirmed that 20 mg/kg QW/Q2W is an appropriate dosing regimen for isatuximab monotherapy for Japanese patients with RRMM, consistent with that used in prior Phase 1/2 monotherapy studies performed in other countries (Martin T G et al., (2014) J Clin Oncol 32:abstract 8532; Martin T et al., (2017) Blood 129: 3294-303). Isatuximab was generally well tolerated and displayed favorable efficacy.

This study indicates that isatuximab monotherapy could be a treatment option for multiple myeloma patients who have received at least three previous lines of therapy, including a PI and an IMiD®, or who are double refractory to a PI and an IMiD®. It showed a favourable safety profile and was well-tolerated even among heavily-treated patients and could therefore be suitable for elderly and frail patients. Responses were observed in patients with high-risk cytogenetics and in patients with more than six prior lines, including patients refractory to both a PI and IMiD®. Heavily pre-treated patients frequently show deteriorations in renal function and bone marrow function due to the primary disease and it is often difficult to continue treatment in such patients for reasons of safety. The current findings are clinically relevant and suggest the possibility of using isatuximab in these patients for whom there may be few alternatives.

Each embodiment herein described may be combined with any other embodiment or embodiments unless clearly indicated to the contrary. In particular, any feature or embodiment indicated as being preferred or advantageous may be combined with any other feature or features or embodiment or embodiments indicated as being preferred or advantageous, unless clearly indicated to the contrary.

All references cited in this application are expressly incorporated by reference herein.

1. (canceled)

2. A method of administering an anti-CD38 antibody to a human individual in need thereof, wherein the individual has multiple myeloma, comprising administering to the individual at least a first dose of the anti-CD38 antibody by intravenous infusion, wherein the anti-CD38 antibody is administered at a dose of at least 10 mg/kg, wherein the dose of the anti-CD38 antibody is in a volume of 250 ml, and wherein the anti-CD38 antibody is isatuximab.

3-4. (canceled)

5. The method of claim 2, wherein the first dose of the anti-CD38 antibody is administered to the individual:
(a) at an infusion rate of 25 mL/hour for a first hour, and wherein the infusion rate is increased by 25 mL/hour every 30 minutes after the first hour to a maximum infusion rate of 150 mL/hour until the 250 ml volume is infused; or
(b) at an infusion rate of 12.5 mL/hour for a first 30 minutes, and wherein the infusion rate is increased by 25 mL/hour every 30 minutes after the first 30 minutes until the 250 ml volume is infused.

6. (canceled)

7. The method of claim 2, comprising administering to the individual at least a second dose of the anti-CD38 antibody by intravenous infusion, wherein the second dose of anti-CD38 antibody is administered to the individual:
(a) at an infusion rate of 50 mL/hour for a first 30 minutes, wherein the infusion rate is increased by 50 ml/hr a second 30 minutes, and wherein the infusion rate is increased by 100 mL/hour every 30 minutes after the second 30 minutes to a maximum infusion rate of 200 mL/hour until the 250 ml volume is infused;
(b) at an infusion rate of 50 mL/hour for a first 30 minutes, 100 mL/hour for a second 30 minutes, 200 mL for the third 30 minutes, and 300 mL/hour after the third 30 minutes until the 250 ml volume is infused; or
(c) at an infusion rate of 25 mL/hour for a first 30 minutes, and wherein the infusion rate is increased by 50 mL/hour every 30 minutes after the first 30 minutes until the 250 ml volume is infused.

8-10. (canceled)

11. The method of claim 7, comprising administering to the individual at least a third dose of the anti-CD38 antibody by intravenous infusion, wherein the third dose of the anti-CD38 antibody is administered to the individual at:
(a) an infusion rate of 200 ml/hour until the 250 ml volume is infused; or
(b) at an infusion rate of 100 ml/hour for a first 30 minutes, and wherein the infusion rate is increased by 50 mL/hour every 30 minutes after the first 30 minutes until the 250 ml volume is infused.

12-13. (canceled)

14. The method of claim 11, comprising administering to the individual a fourth dose of the anti-CD38 antibody by intravenous infusion, wherein the fourth dose of the anti-CD38 antibody is administered is administered to the individual:
(a) at an infusion rate of 200 ml/hour until the 250 ml volume infused; or
(b) at an infusion rate of 100 ml/hour for a first 30 minutes, and wherein the infusion rate is increased by 50 mL/hour every 30 minutes after the first 30 minutes until the 250 ml volume is infused.

15-16. (canceled)

17. The method of claim 14, wherein the anti-CD38 antibody is administered in a first 28-day cycle, wherein the first dose of the anti-CD38 antibody is administered on Day 1, the second dose of the anti-CD38 antibody is administered on Day 8, the third dose of the anti-CD38 antibody is administered on Day 15, and the fourth dose of the anti-CD38 antibody is administered on Day 22 the first 28-day cycle.

18. The method of claim 14, comprising administering to the individual one or more subsequent doses of the anti-CD38 antibody by intravenous infusion following the fourth dose, wherein the one or more subsequent doses of the anti-CD38 antibody are administered to the individual:
(a) at an infusion rate of 200 ml/hour until the 250 ml volume infused; or
(b) at an infusion rate of 100 ml/hour for a first 30 minutes, and wherein the infusion rate is increased by 50 mL/hour every 30 minutes after the first 30 minutes until the 250 ml volume is infused.

19-20. (canceled)

21. The method of claim 18, wherein the anti-CD38 antibody is administered in one or more subsequent 28-day cycles following the first 28-day cycle, wherein each of the one or more subsequent doses of the anti-CD38 antibody following the fourth dose of the anti-CD38 antibody are administered on Days 1 and 15 of each of the one or more subsequent 28-day cycles following the first 28-day cycle.

22. (canceled)

23. A method of administering an anti-CD38 antibody to a human individual in need thereof, wherein the individual has multiple myeloma, comprising administering to the individual at least three doses of the anti-CD38 antibody by intravenous infusion, wherein the anti-CD38 antibody is administered at a dose of at least 10 mg/kg, wherein the dose of the anti-CD38 antibody is in a volume of 250 ml, and wherein the anti-CD38 antibody is isatuximab;
wherein the first dose of the anti-CD38 antibody is administered to the individual at an infusion rate of 25 mL/hour for a first hour, and wherein the infusion rate is increased by 25 mL/hour every 30 minutes after the first hour to a maximum infusion rate of 150 mL/hour until the 250 ml volume is infused;
wherein the second dose of the anti-CD38 is administered to the individual at an infusion rate of 50 mL/hour for a first 30 minutes, wherein the infusion rate is increased by 50 ml/hr a second 30 minutes, and wherein the infusion rate is increased by 100 mL/hour every 30 minutes after the second 30 minutes to a maximum infusion rate of 200 mL/hour until the 250 ml volume is infused; and
wherein the third dose of the anti-CD38 is administered to the individual at an infusion rate of 100 ml/hour for a first 30 minutes, and wherein the infusion rate is increased by 50 mL/hour every 30 minutes after the first 30 minutes until the 250 ml volume is infused.

24-25. (canceled)

26. The method of claim 23, further comprising administering to the individual one or more subsequent doses of the anti-CD38 antibody following the third dose, wherein the one or more subsequent doses of the anti-CD38 antibody are administered to the individual at an infusion rate of 200 ml/hour until the 250 ml volume infused.

27-47. (canceled)

48. A method of safely administering an anti-CD38 antibody to a human individual in need thereof, wherein the individual has multiple myeloma, comprising administering to the individual at least a first dose of the anti-CD38 antibody via intravenous infusion, wherein the anti-CD38 antibody is administered at a dose of at least 10 mg/kg in a volume of 250 ml, wherein the first dose is infused over a duration of about 1.5 and about 6.5 hours, and wherein the anti-CD38 antibody is isatuximab.

49-50. (canceled)

51. The method of claim 48, comprising administering at least a second dose of the anti-CD38 antibody to the individual via intravenous infusion, wherein the anti-CD38 antibody is administered at a dose of at least 10 mg/kg in a volume of 250 ml, wherein the second dose is infused over a duration of about 0.5 and 3.5 hours.

52. The method of claim 51, comprising administering at least a third dose of the anti-CD38 antibody to the individual via intravenous infusion, wherein the anti-CD38 antibody is administered at a dose of at least 10 mg/kg in a volume of 250 ml, wherein the third dose is infused over a duration of about 0.5 and 1.5 hours.

53. The method of claim 7, wherein the rate of infusion reactions (IR) from the intravenous infusion of the second dose of the anti-CD38 antibody is reduced compared to the IR from the intravenous infusion of the first dose of the anti-CD38 antibody.

54-59. (canceled)

60. A method of treating a human individual having multiple myeloma, comprising administering to the individual an anti-CD38 antibody, pomalidomide, and dexamethasone,
wherein the anti-CD38 antibody is isatuximab and is administered in 28-day cycles;
wherein the anti-CD38 antibody is administered on Days 1, 8, 15, and 22 of a first 28-day cycle;
wherein the anti-CD38 antibody is administered on Days 1 and 15 of every 28-day cycle following the first 28-day cycle;
and wherein the anti-CD38 antibody is administered at a dose of 10 mg/kg in a volume of 250 ml or 20 mg/kg.

61-70. (canceled)

71. The method of claim 2, wherein the individual has a respiratory disorder, thoracic disorder, and/or mediastinal disorder.

72. The method of claim 71, wherein the respiratory disorder is chronic obstructive pulmonary disorder (COPD), asthma, or bronchospasm.

73-84. (canceled)

85. An intravenous bag containing between about 360 mg and 1600 mg of an anti-CD38 antibody in a volume of 250 ml, wherein the wherein the anti-CD38 antibody is isatuximab.

86. The method of claim 11, wherein the rate of infusion reactions (IR) from the intravenous infusion of the third dose of the anti-CD38 antibody is reduced compared to the IR from the intravenous infusion of the first dose of the anti-CD38 antibody.

87. The method of claim 14, wherein the rate of infusion reactions (IR) from the intravenous infusion of the fourth dose of the anti-CD38 antibody is reduced compared to the IR from the intravenous infusion of the first dose of the anti-CD38 antibody.
